(19) United States
(12) Patent Application Publication
(10) Pub. No.: US 2024/0130229 A1
(43) Pub. Date: Apr. 18, 2024

(54) High Molecular Compound, Light-Emitting Device, Light-Emitting Apparatus, Electronic Device, and Lighting Device

(71) Applicant: Semiconductor Energy Laboratory Co., Ltd., Kanagawa-ken (JP)

(72) Inventors: Ryo Narukawa, Hadano (JP); Akira Nagasaka, Atsugi (JP); Kunihiko Suzuki, Isehara (JP); Hideko Yoshizumi, Atsugi (JP)

(73) Assignee: Semiconductor Energy Laboratory Co., Ltd., Kanagawa-ken (JP)

(21) Appl. No.: 18/369,543

(22) Filed: Sep. 18, 2023

(30) Foreign Application Priority Data
Sep. 22, 2022 (JP) 2022-151521

(51) Int. Cl. H10K 85/60; C07D 491/048

(52) U.S. Cl. CPC H10K 85/6572; C07D 491/048; H10K 85/626; H10K 2101/20

(57) ABSTRACT

A novel high molecular compound is provided. The high molecular compound includes a repeating unit. The repeating unit has a fluorenediyl group, a hole-transport skeleton, and an electron-transport skeleton. The hole-transport skeleton is bonded to the fluorenediyl group through a substituted or unsubstituted first arylene group. The electron-transport skeleton is bonded to the fluorenediyl group through a substituted or unsubstituted second arylene group. In an excited state, intramolecular charge transfer occurs between the hole-transport skeleton and the electron-transport skeleton.

### BACKGROUND OF THE INVENTION

### 1. Field of the Invention

One embodiment of the present invention relates to a high molecular compound, a light-emitting device, a light-emitting apparatus, an electronic device, and a lighting device. Note that one embodiment of the present invention is not limited to the above technical field. The technical field of one embodiment of the invention disclosed in this specification and the like relates to an object, a method, or a manufacturing method. One embodiment of the present invention relates to a process, a machine, manufacture, or a composition of matter. Specifically, examples of the technical field of one embodiment of the present invention disclosed in this specification include a semiconductor device, a display device, a liquid crystal display device, a light-emitting apparatus, a lighting device, a power storage device, a memory device, an imaging device, a driving method thereof, and a manufacturing method thereof.

### 2. Description of the Related Art

In recent years, light-emitting devices utilizing electroluminescence (EL) have been actively researched and developed. In the basic structure of such a light-emitting device, a layer containing a light-emitting substance (an EL layer) is interposed between a pair of electrodes. Voltage application between the electrodes of this device can cause light emission from the light-emitting substance.

Since the above light-emitting device is a self-luminous device, a display device using this light-emitting device has advantages such as high visibility, no necessity of a backlight, and low power consumption. The display device using the light-emitting device also has advantages in that it can be formed to be thin and lightweight and has high response speed, for example.

In a light-emitting device where an EL layer containing an organic compound as a light-emitting substance is provided between a pair of electrodes (e.g., an organic EL device), voltage application between the pair of electrodes causes injection of electrons from a cathode and holes from an anode into the EL layer having a light-emitting property and thus current flows. By recombination of the injected electrons and holes, the light-emitting organic compound is brought into an excited state to provide light emission.

As the organic compound contained in the light-emitting device, a low molecular compound or a high molecular compound can be used. Since the high molecular compound is thermally stable and can easily form a thin film with excellent uniformity by a coating method or the like, a light-emitting device containing the high molecular compound has been developed (e.g., see Patent Document 1).

Excited states that can be formed by an organic compound are a singlet excited state (S*) and a triplet excited state (T*). Light emission from the singlet excited state is referred to as fluorescence, and light emission from the triplet excited state is referred to as phosphorescence. The statistical generation ratio in the light-emitting device is considered to be S*:T*=1:3. Thus, a light-emitting device containing a compound that emits phosphorescent light (a phosphorescent compound) as a light-emitting substance can have higher emission efficiency than a light-emitting device containing a compound that emits fluorescent light (a fluorescent compound) as a light-emitting substance. Therefore, light-emitting devices containing phosphorescent compounds capable of converting a triplet excited state into light emission have been actively developed in recent years (e.g., see Patent Document 2).

Exciting a host material in a light-emitting device containing a phosphorescent compound requires excitation energy higher than light emission energy of the phosphorescent compound. Note that a large difference between a singlet excitation energy level and a triplet excitation energy level of the host material creates the need for higher excitation energy. The difference between the energy for exciting the host material and the light emission energy of the phosphorescent compound affects device characteristics of a light-emitting device: the driving voltage of the light-emitting device increases. Thus, a method for reducing the driving voltage has been searched (see Patent Document 3).

Among light-emitting devices containing phosphorescent compounds, a light-emitting device that emits blue light in particular has not yet been put into practical use because it is difficult to develop a stable compound having a high triplet excitation energy level. For this reason, the development of a light-emitting device containing a more stable fluorescent compound has been conducted and a technique for increasing the emission efficiency of a light-emitting device containing a fluorescent compound (a fluorescent light-emitting device) has been searched.

As a material capable of partly converting a triplet excited state into light emission, a thermally activated delayed fluorescent (TADF) substance is known in addition to a phosphorescent compound. In a thermally activated delayed fluorescent substance, a singlet excited state is generated from a triplet excited state by reverse intersystem crossing, and light emission from the singlet excited state occurs.

In order to increase the emission efficiency of a light-emitting device containing a thermally activated delayed fluorescent substance, not only efficient generation of a singlet excited state from a triplet excited state but also efficient light emission from a singlet excited state, that is, high fluorescence quantum yield, are important in a thermally activated delayed fluorescent substance. It is, however, not easy to design a light-emitting material that meets these two.

Thus, a method has been proposed in which in a light-emitting device containing a thermally activated delayed fluorescent substance and a fluorescent compound, singlet excitation energy of the thermally activated delayed fluorescent substance is transferred to the fluorescent compound and light emission is obtained from the fluorescent compound (see Patent Document 4).

### REFERENCES

### Patent Documents

[Patent Document 1] Japanese Published Patent Application No. H5-202355
[Patent Document 2] Japanese Published Patent Application No. 2010-182699
[Patent Document 3] Japanese Published Patent Application No. 2012-212879
[Patent Document 4] Japanese Published Patent Application No. 2014-045179

### Non-Patent Documents

[Non-Patent Document 1] Nicholas J. Turro, V. Ramamurthy, J. C. Scaiano, “MODERN MOLECULAR PHOTOCHEMISTRY OF ORGANIC MOLECULES”, UNIVERSITY SCIENCE BOOKS, 2010.02.10, pp. 204-208
[Non-Patent Document 2] Daisaku TANAKA et al., “Ultra High Efficiency Green Organic Light-Emitting Devices”, Japanese Journal of Applied Physics, Vol. 46, No. 1, 2007, pp. L10-L12

### SUMMARY OF THE INVENTION

In order to increase the emission efficiency or to reduce the driving voltage of a light-emitting device containing a light-emitting organic compound, a difference between a singlet excitation energy level and a triplet excitation energy level (ΔEₛₜ) of a host material is preferably small.

In order to increase the emission efficiency of a light-emitting device containing a fluorescent compound, it is preferable that a singlet excited state be efficiently generated from a triplet excited state in a host material. In addition, it is preferable that energy be efficiently transferred from a singlet excited state of the host material to a singlet excited state of the fluorescent compound.

In view of the above, an object of one embodiment of the present invention is to provide a high molecular compound that can be used as a host material. Another object of one embodiment of the present invention is to provide a high molecular compound with small ΔEₛₜ. Another object of one embodiment of the present invention is to provide a light-emitting device that contains a fluorescent compound or a phosphorescent compound and has high emission efficiency. Another object of one embodiment of the present invention is to provide a light-emitting device with low power consumption. Another object of one embodiment of the present invention is to provide a light-emitting apparatus with low power consumption. Another object of one embodiment of the present invention is to provide an electronic device with low power consumption. Another object of one embodiment of the present invention is to provide a lighting device with low power consumption.

Another object of one embodiment of the present invention is to provide a novel high molecular compound. Another object of one embodiment of the present invention is to provide a novel light-emitting device. Another object of one embodiment of the present invention is to provide a novel light-emitting apparatus. Another object of one embodiment of the present invention is to provide a novel electronic device. Another object of one embodiment of the present invention is to provide a novel lighting device.

Note that the description of the above objects does not preclude the existence of other objects. One embodiment of the present invention does not necessarily achieve all the objects listed above. Objects other than the above objects will be apparent from and can be derived from the description of the specification and the like.

One embodiment of the present invention is a high molecular compound including a repeating unit. The repeating unit has a fluorenediyl group, a hole-transport skeleton, and an electron-transport skeleton. The hole-transport skeleton is bonded to the fluorenediyl group through a substituted or unsubstituted first arylene group. The electron-transport skeleton is bonded to the fluorenediyl group through a substituted or unsubstituted second arylene group. Intramolecular charge transfer occurs between the hole-transport skeleton and the electron-transport skeleton in an excited state.

In the high molecular compound having the above structure, it is further preferable that the first arylene group and the second arylene group be bonded to carbon at the 9-position of the fluorenediyl group.

In the high molecular compound having the above structure, it is further preferable that at least one of the first arylene group and the second arylene group be either a substituted or unsubstituted m-phenylene group or a group in which a plurality of substituted or unsubstituted m-phenylene groups are bonded to one another.

Another embodiment of the present invention is a high molecular compound including a repeating unit represented by Formula (G1) below.

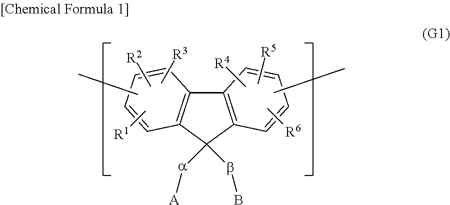

In Formula (G1), α and β each independently represent a substituted or unsubstituted arylene group having 6 to 24 carbon atoms, A represents a hole-transport skeleton, B represents an electron-transport skeleton, and R¹ to R⁶ each independently represent any one of hydrogen (including deuterium), a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms.

In the high molecular compound having the above structure, it is further preferable that α and β each independently represent either a substituted or unsubstituted m-phenylene group or a group in which a plurality of substituted or unsubstituted m-phenylene groups are bonded to one another.

In the high molecular compound having any of the above structures, it is further preferable that the hole-transport skeleton have at least one of a π-electron rich heteroaromatic skeleton and an aromatic amine skeleton and the electron-transport skeleton have a π-electron deficient heteroaromatic skeleton.

Another embodiment of the present invention is a light-emitting device containing a light-emitting substance and the high molecular compound having any of the above structures.

Another embodiment of the present invention is a light-emitting apparatus including the above light-emitting device and at least one of a transistor and a substrate.

Another embodiment of the present invention is an electronic device including the above light-emitting apparatus and at least one of a sensing portion, an input portion, and a communication portion.

Another embodiment of the present invention is a lighting device including the above light-emitting apparatus and a housing.

In addition, a light-emitting apparatus includes, in its category, a module in which a light-emitting apparatus is connected to a connector such as a flexible printed circuit (FPC) or a tape carrier package (TCP), a module in which a printed wiring board is provided on the tip of a TCP, and a module in which an integrated circuit (IC) is directly mounted on a light-emitting device by a chip on glass (COG) method.

One embodiment of the present invention can provide a high molecular compound that can be used as a host material. Another embodiment of the present invention can provide a high molecular compound with small ΔEₛₜ. Another embodiment of the present invention can provide a light-emitting device that contains a fluorescent compound or a phosphorescent compound and has high emission efficiency. Another embodiment of the present invention can provide a light-emitting device with low power consumption. Another embodiment of the present invention can provide a light-emitting apparatus with low power consumption. Another embodiment of the present invention can provide an electronic device with low power consumption. Another embodiment of the present invention can provide a lighting device with low power consumption.

One embodiment of the present invention can provide a novel high molecular compound. One embodiment of the present invention can provide a novel light-emitting device. One embodiment of the present invention can provide a novel light-emitting apparatus. One embodiment of the present invention can provide a novel display device. One embodiment of the present invention can provide a novel electronic device. One embodiment of the present invention can provide a novel lighting device.

Note that the description of these effects does not preclude the existence of other effects. One embodiment of the present invention does not necessarily have all these effects. Other effects will be apparent from and can be derived from the description of the specification, the drawings, the claims, and the like.

### BRIEF DESCRIPTION OF THE DRAWINGS

In the accompanying drawings:

FIG. 1A is a schematic cross-sectional view of a light-emitting device of one embodiment of the present invention, FIG. 1B is a schematic cross-sectional view of a light-emitting layer of one embodiment of the present invention, and FIG. 1C shows a correlation between energy levels in the light-emitting layer;

FIG. 2A is a schematic cross-sectional view of a light-emitting layer of one embodiment of the present invention, and FIG. 2B shows a correlation between energy levels in the light-emitting layer;

FIGS. 3A to 3E each illustrate a structure of a light-emitting device of an embodiment;

FIGS. 4A to 4D illustrate a light-emitting apparatus of an embodiment;

FIGS. 5A to 5C illustrate a fabrication method of a light-emitting apparatus of an embodiment;

FIGS. 6A to 6C illustrate a fabrication method of a light-emitting apparatus of an embodiment;

FIGS. 7A to 7D illustrate a fabrication method of a light-emitting apparatus of an embodiment;

FIGS. 8A to 8C illustrate a light-emitting apparatus of an embodiment;

FIGS. 9A to 9F each illustrate a light-emitting apparatus of an embodiment;

FIGS. 10A and 10B illustrate a light-emitting apparatus of an embodiment;

FIGS. 11A to 11E illustrate electronic devices of an embodiment;

FIGS. 12A to 12E illustrate electronic devices of an embodiment;

FIGS. 13A and 13B illustrate electronic devices of an embodiment;

FIGS. 14A and 14B illustrate a lighting device of an embodiment;

FIG. 15 illustrates lighting devices of an embodiment;

FIGS. 16A to 16D show calculation analysis results of Dimer A; and

FIGS. 17A to 17D show calculation analysis results of Dimer B.

### DETAILED DESCRIPTION OF THE INVENTION

Embodiments of the present invention will be described in detail below with reference to the drawings. Note that the present invention is not limited to the following description, and the modes and details of the present invention can be modified in various ways without departing from the spirit and scope of the present invention. Therefore, the present invention should not be construed as being limited to the description in the following embodiments.

Note that the position, size, range, or the like of each component illustrated in drawings and the like is not accurately represented in some cases for easy understanding. Therefore, the disclosed invention is not necessarily limited to the position, size, range, or the like disclosed in the drawings and the like.

Ordinal numbers such as “first” and “second” in this specification and the like are used for convenience and do not denote the order of steps or the stacking order of layers in some cases. Therefore, for example, description can be made even when “first” is replaced with “second” or “third”, as appropriate. In addition, the ordinal numbers in this specification and the like are not necessarily the same as those used to specify one embodiment of the present invention.

In the description of modes of the invention in this specification and the like with reference to the drawings, the same components in different drawings are denoted by the same reference numeral in some cases.

In this specification and the like, the terms “film” and “layer” can be interchanged with each other. For example, the term “conductive layer” can be changed to the term “conductive film” in some cases. For another example, the term “insulating film” can be changed into the term “insulating layer” in some cases.

In this specification and the like, a singlet excited state (S*) refers to a singlet state having excitation energy. An S₁ level means the lowest level of the singlet excitation energy level, that is, the excitation energy level of the lowest singlet excited state. A triplet excited state (T*) refers to a triplet state having excitation energy. A T₁ level means the lowest level of the triplet excitation energy level, that is, the excitation energy level of the lowest triplet excited state.

In this specification and the like, a fluorescent material or a fluorescent compound refers to a material or a compound that emits light when the relaxation from the singlet excited state to the ground state occurs. A phosphorescent material or a phosphorescent compound refers to a material or a compound that emits light in the visible light region at room temperature when the relaxation from the triplet excited state to the ground state occurs. That is, a phosphorescent material or a phosphorescent compound refers to a material or a compound that can convert triplet excitation energy into visible light.

When the ν=0→ν=0 transition (0→0 band) between vibrational levels of the ground state and the excited state is clearly observed from a fluorescent spectrum or a phosphorescent spectrum, the S₁ level or the T₁ level of an organic compound is preferably calculated using the 0→0 band (see Non-Patent Document 1, for example). When the 0→0 band is unclear, the S₁ level can be energy of the wavelength at the intersection of the horizontal axis (wavelength) or the base line and a tangent to the fluorescent spectrum at a point where the slope of the spectrum at a peak on the shorter wavelength side has a maximum value and the T₁ level can be energy of the wavelength at the intersection of the horizontal axis (wavelength) or the base line and a tangent to the phosphorescent spectrum at a point where the slope of the spectrum at a peak on the shorter wavelength side has a maximum value (see Non-Patent Document 2, for example). In the case where the levels are compared with each other, those calculated by the same method are used.

Note that in this specification and the like, “room temperature” refers to a temperature in a range of 0° C. to 40° C.

In this specification and the like, a high molecular material or a high molecular compound refers to a polymer that has molecular weight distribution and an average molecular weight of 1×10³ to 1×10⁸. A low molecular material or a low molecular compound refers to a material or a compound that does not have molecular weight distribution and whose molecular weight is less than or equal to 1×10⁴.

A high molecular material or a high molecular compound refers to a material or a compound in which one or more repeating units are polymerized. In other words, the repeating unit refers to a unit including one or more high molecular materials or one or more high molecular compounds.

A high molecular material or a high molecular compound may refer to a block copolymer, a random copolymer, an alternating copolymer, a graft copolymer, or the like.

In the case where an end group of a high molecular material or a high molecular compound includes a polymerization active group, the emission characteristics or luminance lifetime of a light-emitting device might be reduced. For this reason, an end group of a high molecular material or a high molecular compound is preferably a stable end group. As the stable end group, a group that is covalently bonded to a main chain is preferable, and a group that is bonded to an aryl group or a heterocyclic group through a carbon-carbon bond is particularly preferable.

In this specification and the like, a blue wavelength range refers to a wavelength range of greater than or equal to 400 nm and less than 490 nm, and blue light emission has at least one emission spectrum peak in that range. A green wavelength range refers to a wavelength range of greater than or equal to 490 nm and less than 580 nm, and green light emission has at least one emission spectrum peak in that range. A red wavelength range refers to a wavelength range of greater than or equal to 580 nm and less than or equal to 680 nm, and red light emission has at least one emission spectrum peak in that range.

### Embodiment 1

In this embodiment, a high molecular compound of one embodiment of the present invention will be described.

One embodiment of the present invention is a high molecular compound having a fluorenediyl group as a main chain skeleton, i.e., a polyfluorene derivative. A polyfluorene derivative is known as a conductive high molecule and has a structure preferred for a light-emitting device.

One embodiment of the present invention is a high molecular compound having a structure including a hole-transport skeleton and an electron-transport skeleton as side chains.

That is, one embodiment of the present invention is a high molecular compound including a repeating unit. The repeating unit has a fluorenediyl group, a hole-transport skeleton, and an electron-transport skeleton. The hole-transport skeleton is bonded to the fluorenediyl group through a substituted or unsubstituted first arylene group. The electron-transport skeleton is bonded to the fluorenediyl group through a substituted or unsubstituted second arylene group. In an excited state, intramolecular charge transfer occurs between the electron-transport skeleton and the hole-transport skeleton.

In one embodiment of the present invention, the repeating unit preferably has a three-dimensional structure in which the hole-transport skeleton and the electron-transport skeleton are stacked by π-π interaction. This offers a molecular configuration that facilitates the intramolecular charge transfer between the electron-transport skeleton and the hole-transport skeleton in the same repeating unit in the excited state. Accordingly, the efficiency of the intramolecular charge transfer in the excited state in the high molecular compound of one embodiment of the present invention can be improved.

In one embodiment of the present invention, highest occupied molecular orbital (also referred to as HOMO) distribution in the repeating unit can be easily located on the hole-transport skeleton, and lowest unoccupied molecular orbital (also referred to as LUMO) distribution in the repeating unit can be easily located on the electron-transport skeleton. Thus, the HOMO and the LUMO in the repeating unit are spatially separated from each other and an overlap between the HOMO and the LUMO in the repeating unit is extremely small. This can improve the efficiency of the intramolecular charge transfer in the excited state in the high molecular compound of one embodiment of the present invention.

Accordingly, in the high molecular compound of one embodiment of the present invention, the efficiency of the intramolecular charge transfer is improved and thus exchange interaction is small, so that a difference (ΔEₛₜ) between a singlet excitation energy level and a triplet excitation energy level is small. Hence, the use of the high molecular compound of one embodiment of the present invention as a host material in a light-emitting device can increase its emission efficiency or can reduce its driving voltage. In the high molecular compound of one embodiment of the present invention, ΔEₛₜ is preferably greater than 0 eV and less than or equal to 0.2 eV. Note that the molecular orbital refers to a region where an electron is highly probably found. With the molecular orbital, the electron configuration of the molecule (the spatial distribution and energy of electrons) can be described in detail.

In the high molecular compound of one embodiment of the present invention, the intramolecular charge transfer in the excited state refers to, for example, intramolecular charge transfer in a singlet excited state and intramolecular charge transfer in a triplet excited state. In the high molecular compound of one embodiment of the present invention, intramolecular charge transfer preferably occurs in one or both of the singlet excited state and the triplet excited state. Note that in the high molecular compound of one embodiment of the present invention, the intramolecular charge transfer in the excited state is not limited to the intramolecular charge transfer in the singlet excited state and the intramolecular charge transfer in the triplet excited state.

In the high molecular compound of one embodiment of the present invention, charge transfer that occurs in the excited state is not limited to intramolecular charge transfer and may include intermolecular charge transfer. That is, an exciplex (also referred to as an excited dimer or an excimer) may be formed in two high molecular chains in a high molecular compound 131.

As described above, the hole-transport skeleton and the electron-transport skeleton are preferably stacked by the π-π interaction in one embodiment of the present invention. Thus, in one embodiment of the present invention, it is further preferable that both of the first arylene group bonded to the hole-transport skeleton and the second arylene group bonded to the electron-transport skeleton be bonded to carbon at the 9-position of the fluorenediyl group. This makes the hole-transport skeleton and the electron-transport skeleton in the same repeating unit close to each other. Accordingly, the hole-transport skeleton and the electron-transport skeleton are easily stacked by π-π interaction, which can further improve the efficiency of the intramolecular charge transfer.

That is, one embodiment of the present invention is specifically a high molecular compound including a repeating unit represented by Formula (G1) below. The structure in Formula (G1) can facilitate intramolecular charge transfer between an electron-transport skeleton and a hole-transport skeleton in one repeating unit in an excited state.

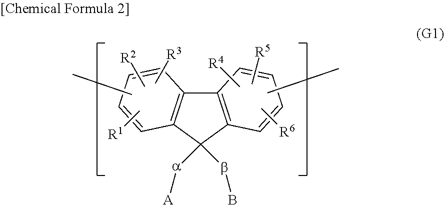

In Formula (G1), α and β each independently represent a substituted or unsubstituted arylene group having 6 to 24 carbon atoms, A represents the hole-transport skeleton, B represents the electron-transport skeleton, and R¹ to R⁶ each independently represent any one of hydrogen (including deuterium), a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms.

Another specific example of one embodiment of the present invention is a high molecule having a structure represented by Formula (G2) below.

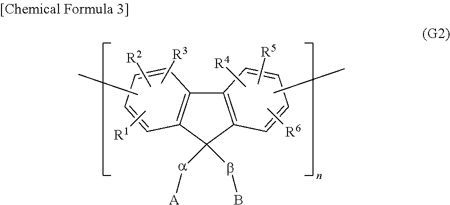

In Formula (G2), α and β each independently represent a substituted or unsubstituted arylene group having 6 to 24 carbon atoms, A represents a hole-transport skeleton, B represents an electron-transport skeleton, R¹ to R⁶ each independently represent any one of hydrogen (including deuterium), a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms, and n is an integer greater than or equal to 1 and less than or equal to 10000. As the end of the structure, for example, halogen, hydrogen, or the like can be used.

In Formulae (G1) and (G2), α corresponds to the first arylene group and β corresponds to the second arylene group. In the case of simply describing α below, the content can apply to the first arylene group. In the case of simply describing β below, the content can apply to the second arylene group.

As the hole-transport skeleton in one embodiment of the present invention, specifically, a skeleton having at least one of an aromatic amine skeleton and a π-electron rich heteroaromatic skeleton can be used.

As the aromatic amine skeleton, a tertiary amine, which does not include an NH bond, is preferable, and a triarylamine skeleton is particularly preferable. As an aryl group of a triarylamine skeleton, a substituted or unsubstituted aryl group having 6 to 13 carbon atoms in a ring is preferable and examples of the aryl group include a phenyl group, a naphthyl group, and a fluorenyl group.

As the π-electron rich heteroaromatic skeleton, one or more of a furan skeleton, a thiophene skeleton, and a pyrrole skeleton are preferable because of their high stability and reliability. A dibenzofuran skeleton is preferable as a furan skeleton, and a dibenzothiophene skeleton is preferable as a thiophene skeleton. As a pyrrole skeleton, an indole skeleton, a carbazole skeleton, and a 3-(9-phenyl-9H-carbazol-3-yl)-9H-carbazole skeleton are particularly preferable. Each of these skeletons may have a substituent.

Specific examples of the hole-transport skeleton include aromatic amine skeletons represented by Formulae (A-1) and (A-2) below and π-electron rich heteroaromatic skeletons represented by Formulae (A-3) to (A-37) below. In General Formulae (A-1) to (A-37), Q represents an oxygen atom or a sulfur atom, γ represents an arylene group having 6 to 24 carbon atoms, and Ar¹ and Ar² each independently represent an aryl group having 6 to 18 carbon atoms. Although Formulae (A-1) to (A-37) below are specific examples of the aromatic amine skeleton and the π-electron rich heteroaromatic skeleton in one embodiment of the present invention, the present invention is not limited thereto.

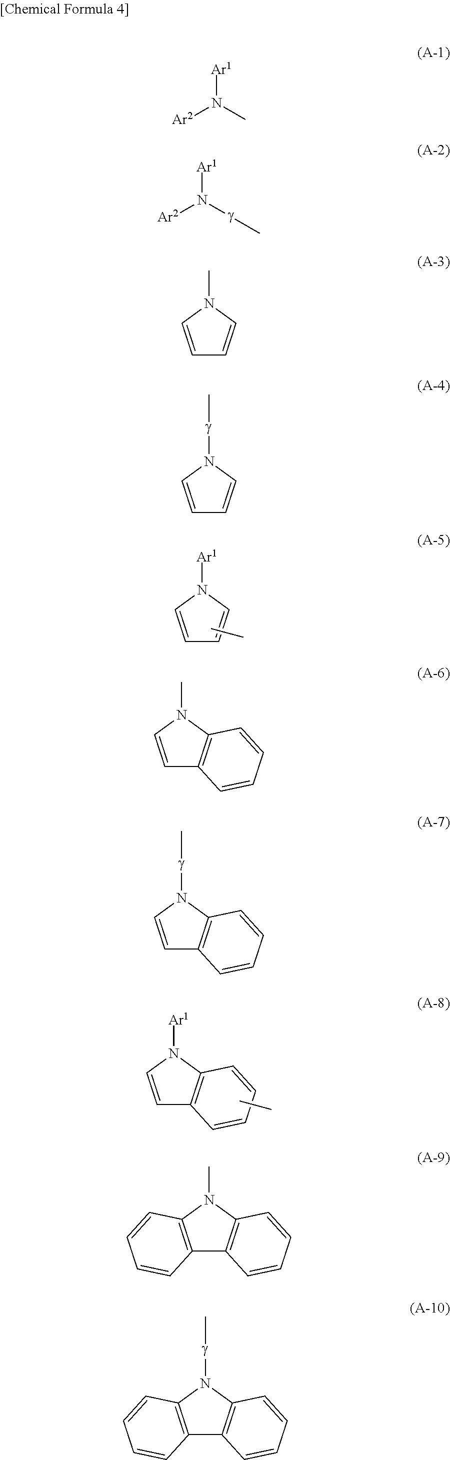

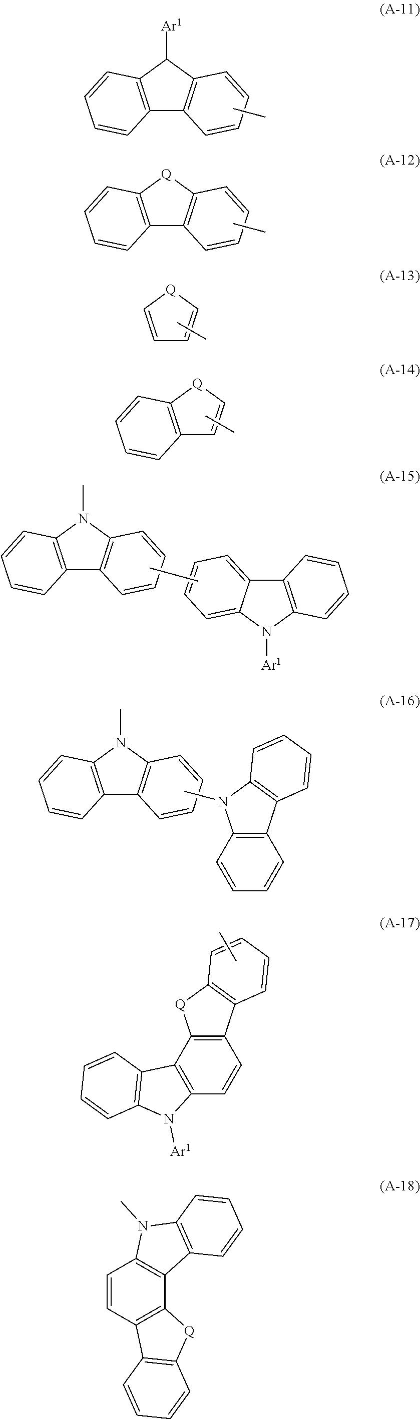

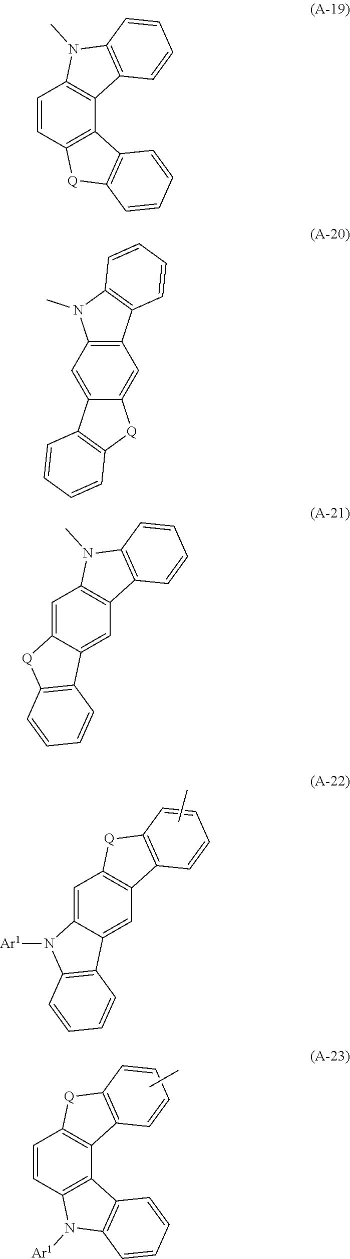

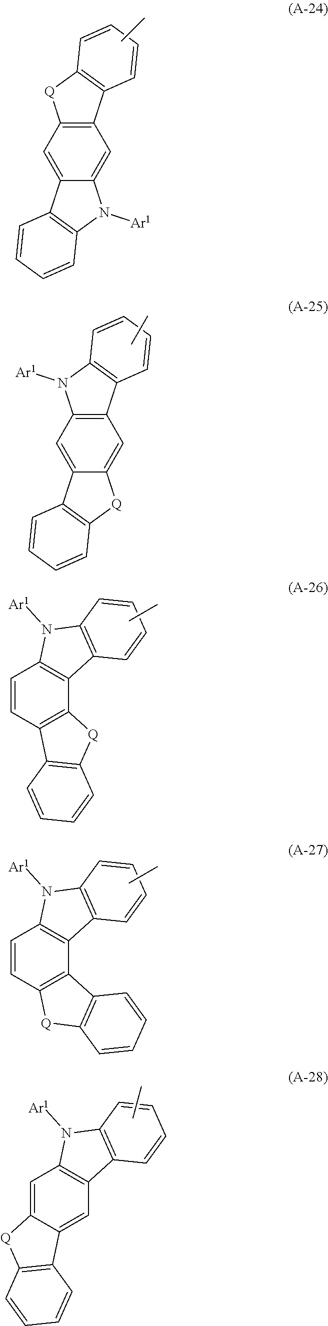

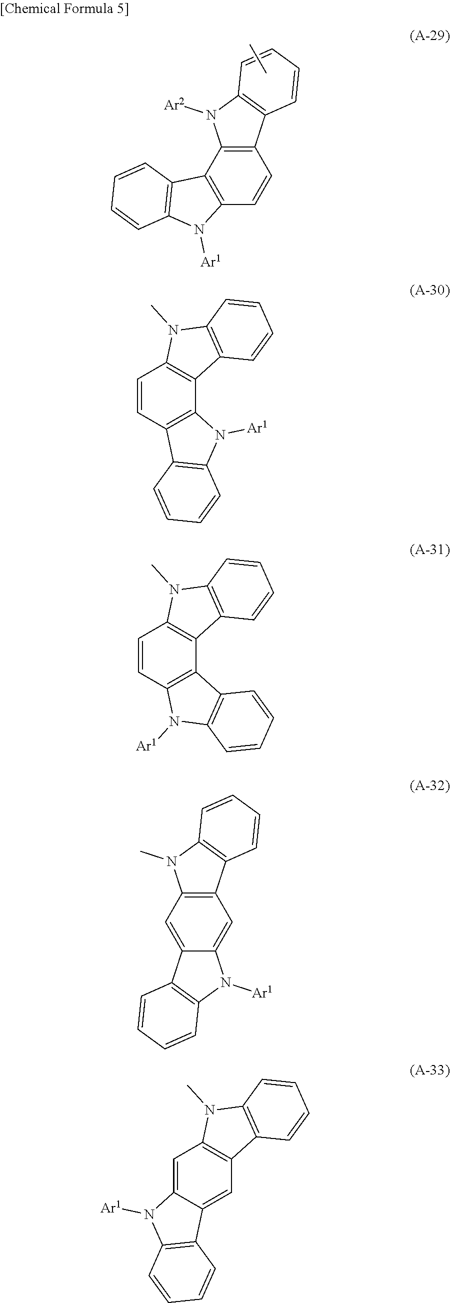

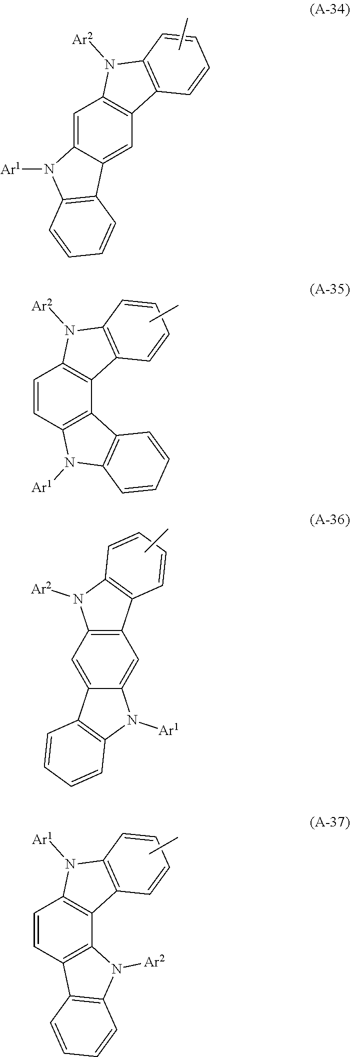

In the hole-transport skeleton, the aromatic amine skeleton or the π-electron rich heteroaromatic skeleton may have a substituent, and specific examples of the substituent include an alkyl group having 1 to 6 carbon atoms and an aryl group having 6 to 18 carbon atoms. Moreover, as the substituent of the π-electron rich heteroaromatic skeleton, a π-electron deficient heteroaromatic skeleton described later may be used. Hydrogen contained in the aromatic amine skeleton or the π-electron rich heteroaromatic skeleton may be deuterium.

In one embodiment of the present invention, a π-electron deficient heteroaromatic skeleton can be used as the electron-transport skeleton. As the π-electron deficient heteroaromatic skeleton, a pyridine skeleton, a diazine skeleton (a pyrimidine skeleton, a pyrazine skeleton, or a pyridazine skeleton), or a triazine skeleton is preferable; in particular, the diazine skeleton or the triazine skeleton is preferable because of its high stability and reliability.

Specific examples of the π-electron deficient heteroaromatic skeleton include skeletons represented by Formulae (B-1) to (B-39) below. In General Formulae (B-1) to (B-39), Q represents an oxygen atom or a sulfur atom, γ represents an arylene group having 6 to 24 carbon atoms, and Ar¹ represents an aryl group having 6 to 18 carbon atoms. Although Formulae (B-1) to (B-39) below are specific examples of the π-electron deficient heteroaromatic skeleton in one embodiment of the present invention, the present invention is not limited thereto.

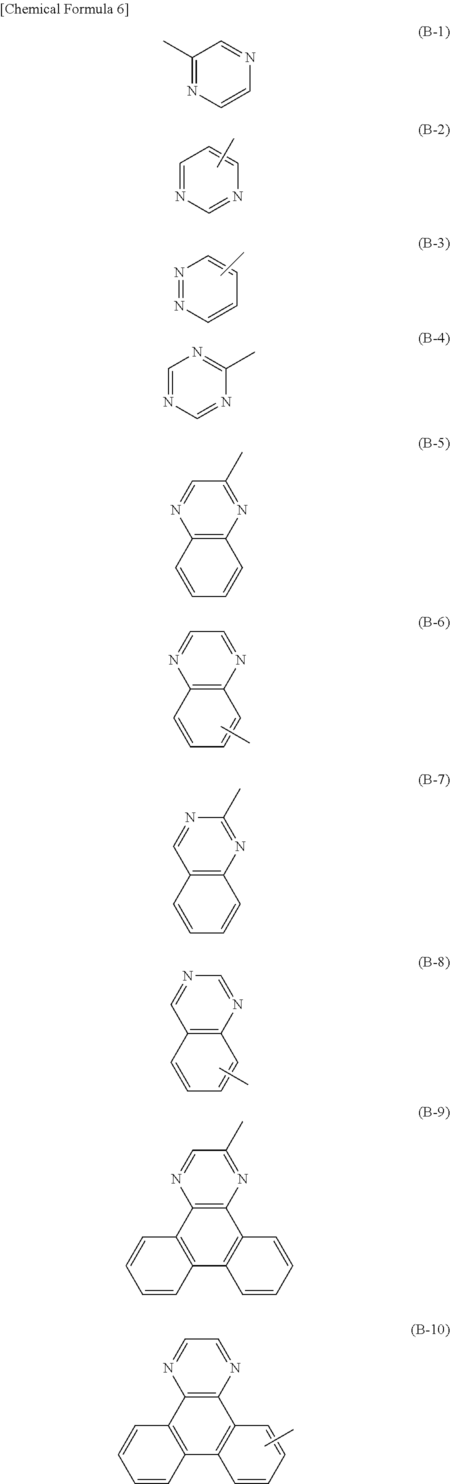

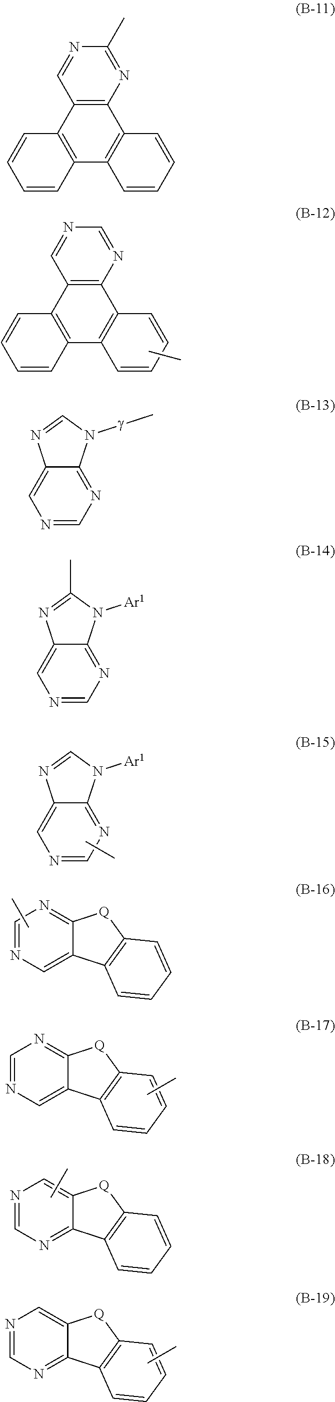

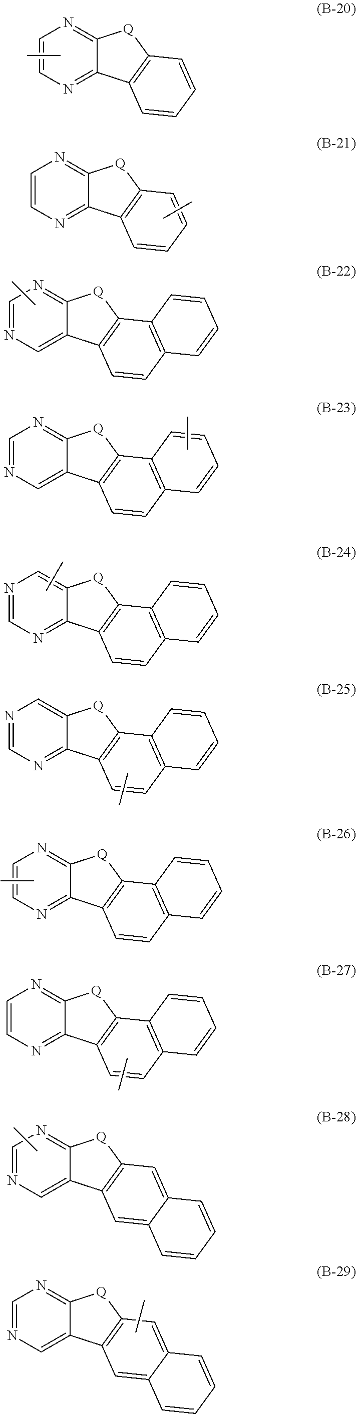

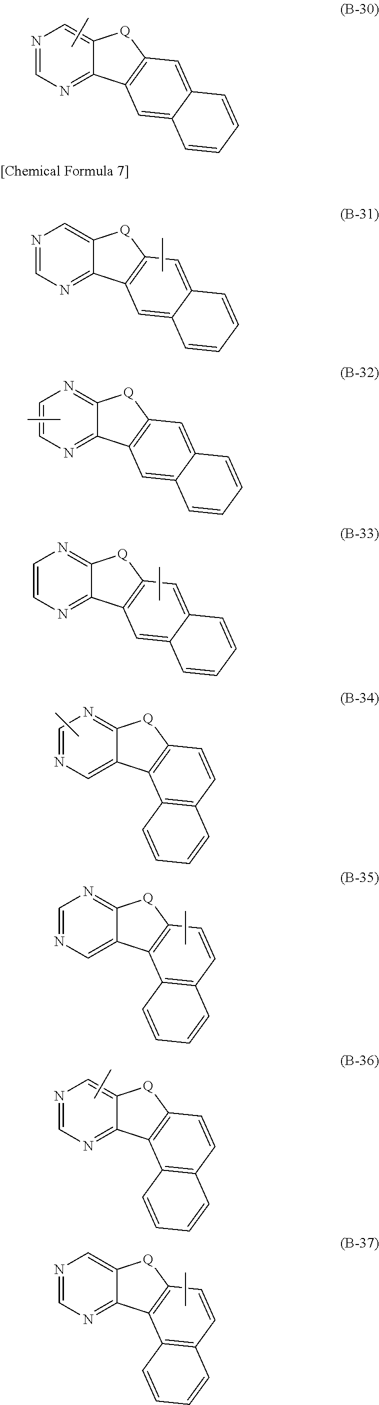

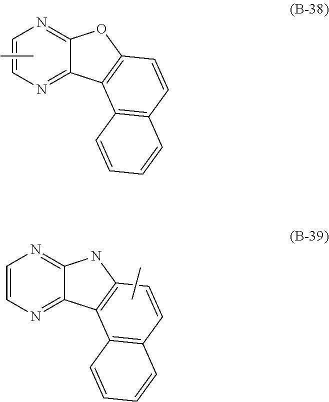

In the electron-transport skeleton, the π-electron deficient heteroaromatic skeleton may have a substituent, and specific examples of the substituent include an alkyl group having 1 to 6 carbon atoms and an aryl group having 6 to 18 carbon atoms. Moreover, as the substituent of the π-electron deficient heteroaromatic skeleton, the above-described π-electron rich heteroaromatic skeleton may be used. Hydrogen contained in the π-electron deficient heteroaromatic skeleton may be deuterium.

In one embodiment of the present invention, specific examples of the arylene group having 6 to 24 carbon atoms include an o-phenylene group, an m-phenylene group, a p-phenylene group, a 1,5-naphthylene group, a 1,4-naphthylene group, a 9,9-dimethylfluorene-2,7-diyl group, a 4,4-biphenylene group, a benzo[a]phenanthrenylene group, a benzo[c]phenanthrenylene group, a terphenylene group, and a quaterphenylene group. Note that the arylene group having 6 to 24 carbon atoms may have a substituent, and specific examples of the substituent include an alkyl group having 1 to 6 carbon atoms and an aryl group having 6 to 18 carbon atoms. Hydrogen contained in the substituted or unsubstituted arylene group having 6 to 24 carbon atoms may be deuterium.

In one embodiment of the present invention, it is preferable that one of α (the first arylene group) and β (the second arylene group), further preferably both of them, be a substituted or unsubstituted m-phenylene group or a group in which a plurality of substituted or unsubstituted m-phenylene groups are bonded to one another.

In the case where α (the first arylene group) is a p-phenylene group or a group in which a plurality of p-phenylene groups are bonded to one another in one embodiment of the present invention, for example, a π-electron conjugated system easily spreads between the hole-transport skeleton and α. This might reduce the efficiency of intramolecular charge transfer between the electron-transport skeleton and the hole-transport skeleton in the excited state.

In the case where β (the second arylene group) is a p-phenylene group or a group in which a plurality of p-phenylene groups are bonded to one another in one embodiment of the present invention, a π-electron conjugated system spreads between the electron-transport skeleton and β. This might reduce the efficiency of intramolecular charge transfer between the electron-transport skeleton and the hole-transport skeleton in the excited state.

Meanwhile, in the case where α (the first arylene group) is a substituted or unsubstituted m-phenylene group or a group in which a plurality of substituted or unsubstituted m-phenylene groups are bonded to one another, the positional relationship between the hole-transport skeleton and α is three-dimensionally twisted. This can inhibit a π-electron conjugated system from spreading between the hole-transport skeleton and α, which can prevent a reduction in the efficiency of the intramolecular charge transfer between the electron-transport skeleton and the hole-transport skeleton in the excited state.

In the case where β (the second arylene group) is a substituted or unsubstituted m-phenylene group or a group in which a plurality of substituted or unsubstituted m-phenylene groups are bonded to one another, the positional relationship between the electron-transport skeleton and β is three-dimensionally twisted. This can inhibit a π-electron conjugated system from spreading between the electron-transport skeleton and β, which can prevent a reduction in the efficiency of the intramolecular charge transfer between the electron-transport skeleton and the hole-transport skeleton in the excited state.

Preferred specific examples of α (the first arylene group) and β (the second arylene group) include skeletons represented by Formulae (L-1) to (L-18) below. Although Formulae (L-1) to (L-18) below are preferred specific examples of α and β in one embodiment of the present invention, the present invention is not limited thereto.

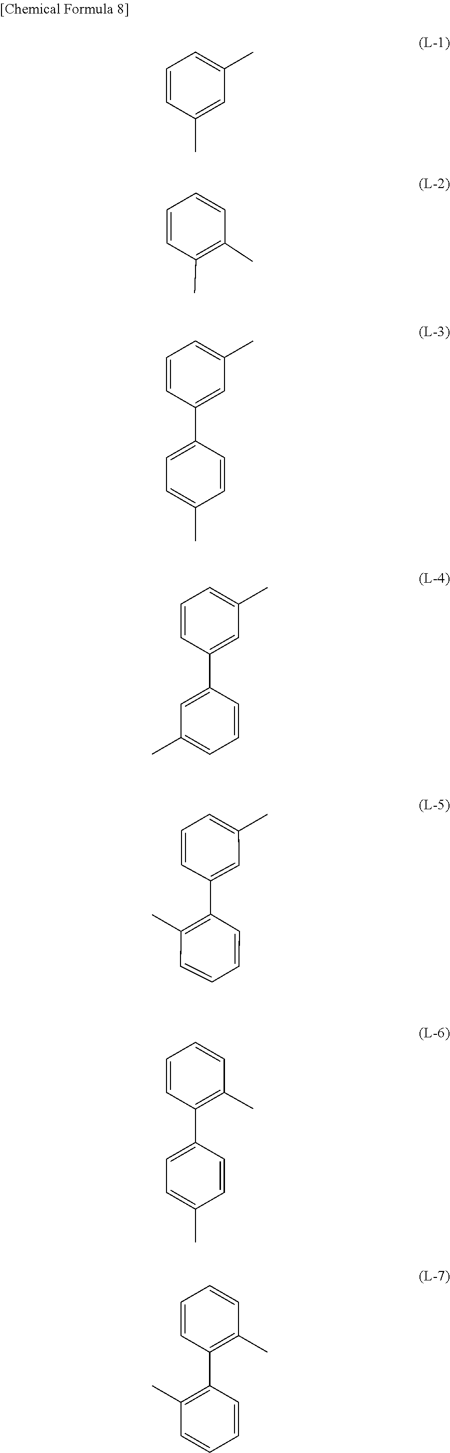

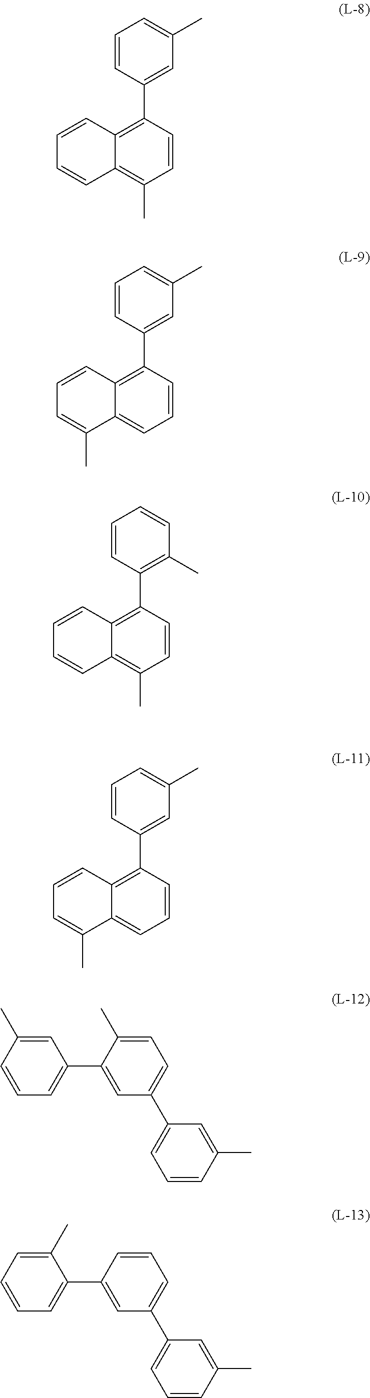

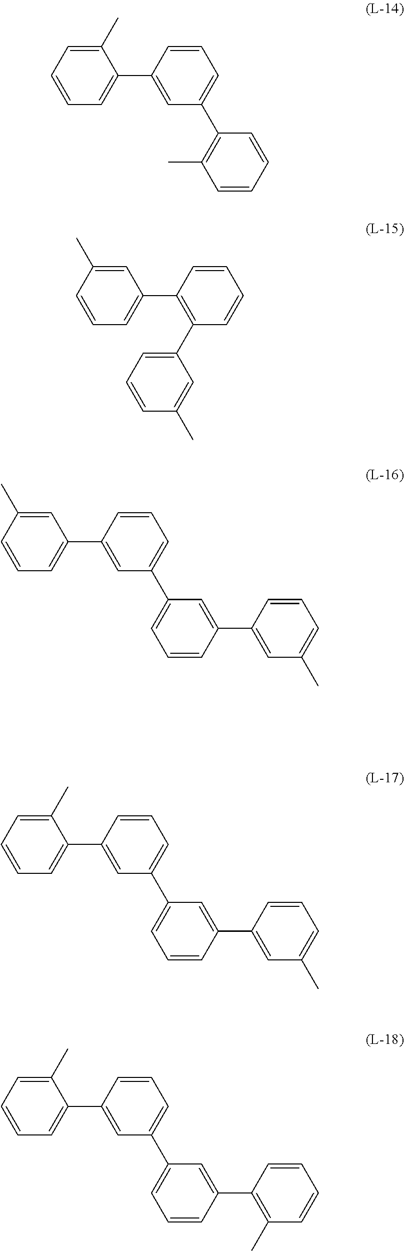

Note that α (the first arylene group) and β (the second arylene group) may each have a substituent, and specific examples of the substituent include an alkyl group having 1 to 6 carbon atoms and a substituted or unsubstituted phenyl group. Hydrogen contained in α (the first arylene group) and β (the second arylene group) may be deuterium.

Specific examples of the aryl group having 6 to 18 carbon atoms in the high molecular compound of one embodiment of the present invention include a phenyl group, an o-tolyl group, an m-tolyl group, a p-tolyl group, a mesityl group, an o-biphenyl group, an m-biphenyl group, a p-biphenyl group, a 1-naphthyl group, a 2-naphthyl group, a fluorenyl group, and a triphenylenyl group.

Specific examples of the straight-chain alkyl group having 1 to 10 carbon atoms and the branched alkyl group having 3 to 10 carbon atoms in the high molecular compound of one embodiment of the present invention include a methyl group, an ethyl group, an n-propyl group, an isopropyl group, an n-butyl group, an isobutyl group, a tert-butyl group, a sec-butyl group, a neopentyl group, an n-hexyl group, an n-octyl group, and an n-decyl group.

Specific examples of the alkoxy group having 1 to 10 carbon atoms in the high molecular compound of one embodiment of the present invention include a methoxy group, an ethoxy group, a propoxy group, a t-butoxy group, a pentyloxy group, an octyloxy group, an allyloxy group, a cyclohexyloxy group, a phenoxy group, and a benzyloxy group.

Specific examples of the cycloalkyl group having 3 to 12 carbon atoms in the high molecular compound of one embodiment of the present invention include a cyclopropyl group, a cyclobutyl group, a cyclopentyl group, a cyclohexyl group, a cycloheptyl group, a cyclooctyl group, a cyclononanyl group, a cyclodecyl group, and a cyclododecyl group.

Specific examples of the cycloalkyl group having abridged structure and having 4 to 10 carbon atoms in the high molecular compound of one embodiment of the present invention include a bicyclobutyl group, a noradamantyl group, an adamantyl group, a norbornanyl group, and a tetrahydrodicyclopentadienyl group.

Specific examples of the trialkylsilyl group having 3 to 12 carbon atoms in the high molecular compound of one embodiment of the present invention include a trimethylsilyl group, a triethylsilyl group, and a tert-butyl dimethylsilyl group.

Specific examples of the repeating unit represented by General Formula (G1) include Structural Formulae (100) to (111) below. Although Structural Formulae (100) to (111) below are specific examples of the repeating unit represented by General Formula (G1), the present invention is not limited thereto.

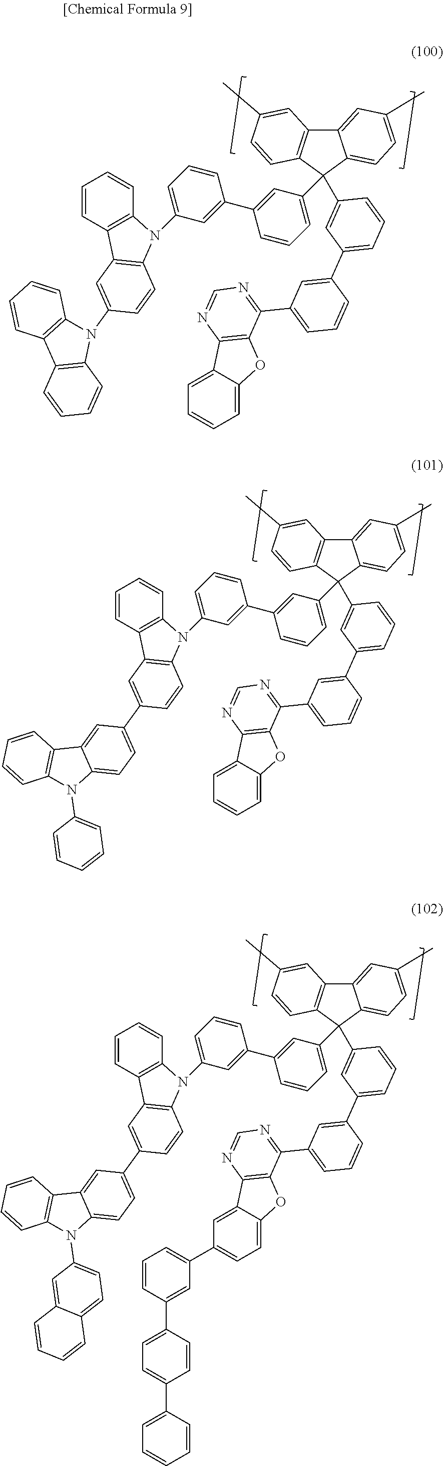

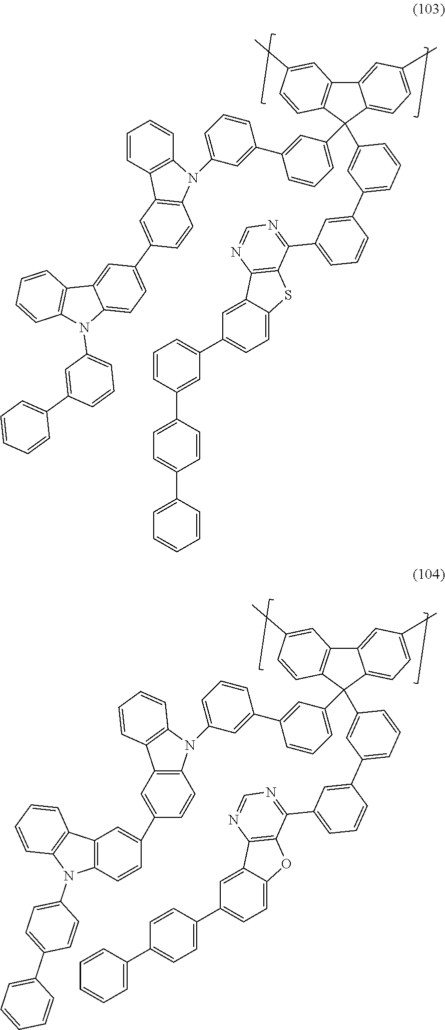

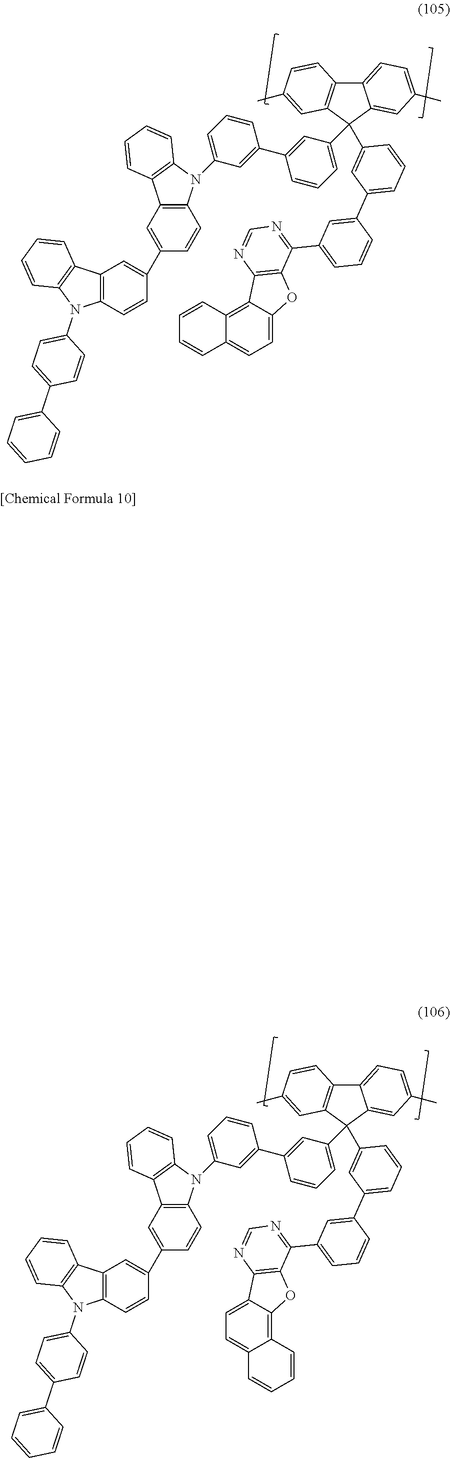

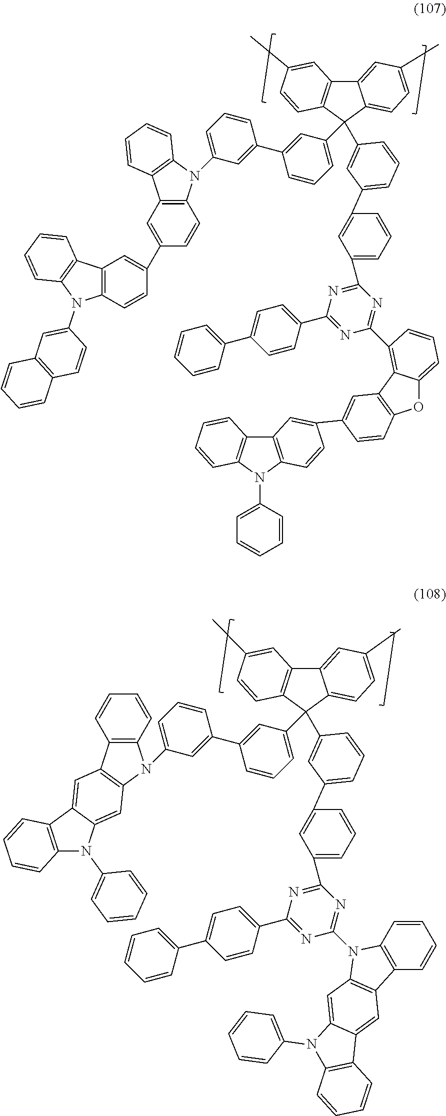

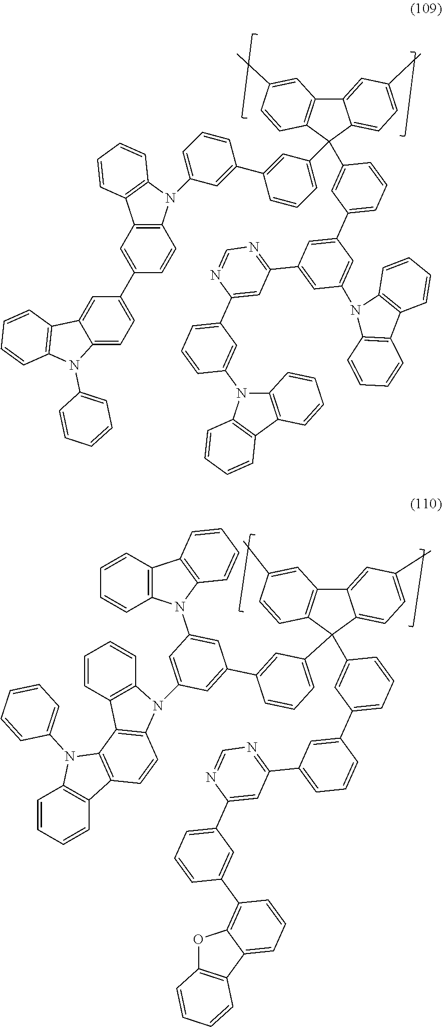

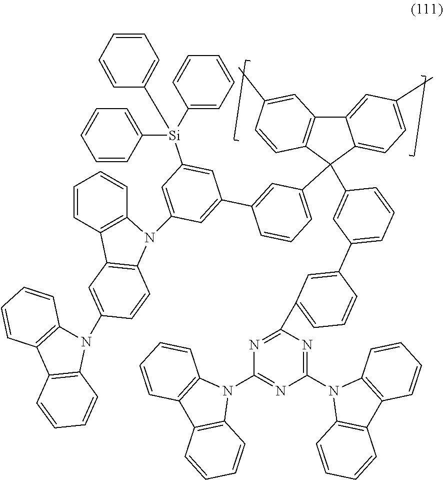

Next, a method for synthesizing the high molecular compound including the repeating unit represented by General Formula (G1) above will be described. First, a method for synthesizing an organic compound represented by General Formula (G0) below, which is a monomer that can be used for synthesis of the high molecular compound, will be described.

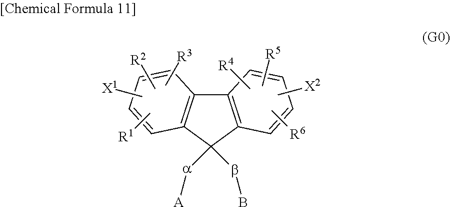

In Formula (G0), α and β each independently represent a substituted or unsubstituted arylene group having 6 to 24 carbon atoms, A represents a hole-transport skeleton, B represents an electron-transport skeleton, and R¹ to R⁶ each independently represent any one of hydrogen (including deuterium), a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms. Furthermore, X¹ and X² each independently represent halogen.

As shown in Scheme (a-1) below, for example, the organic compound represented by General Formula (G0) can be obtained in the following manner: an organic compound represented by Formula (g0) is heated in the presence of acetic acid and an acid catalyst, or boron trifluoride diethyl ether is added to the organic compound represented by Formula (g0) in a solvent such as dichloromethane, the resulting mixture is stirred, and intramolecular dehydration is performed.

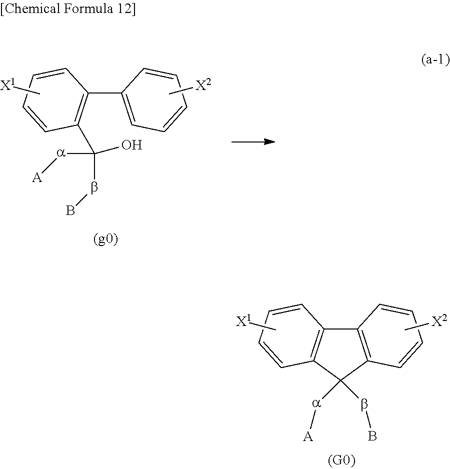

In Formula (g0), a and 3 each independently represent a substituted or unsubstituted arylene group having 6 to 24 carbon atoms, A represents a hole-transport skeleton, B represents an electron-transport skeleton, and R¹ to R⁶ each independently represent any one of hydrogen (including deuterium), a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms. Furthermore, X¹ and X² each independently represent halogen.

Specific examples of the organic compound represented by General Formula (G0) include Structural Formulae (100m) to (111m) below. Although Structural Formulae (100m) to (111m) below are specific examples of the organic compound represented by General Formula (G0), the present invention is not limited thereto. In Structural Formulae (100m) to (111m) below, X¹ and X² each independently represent halogen.

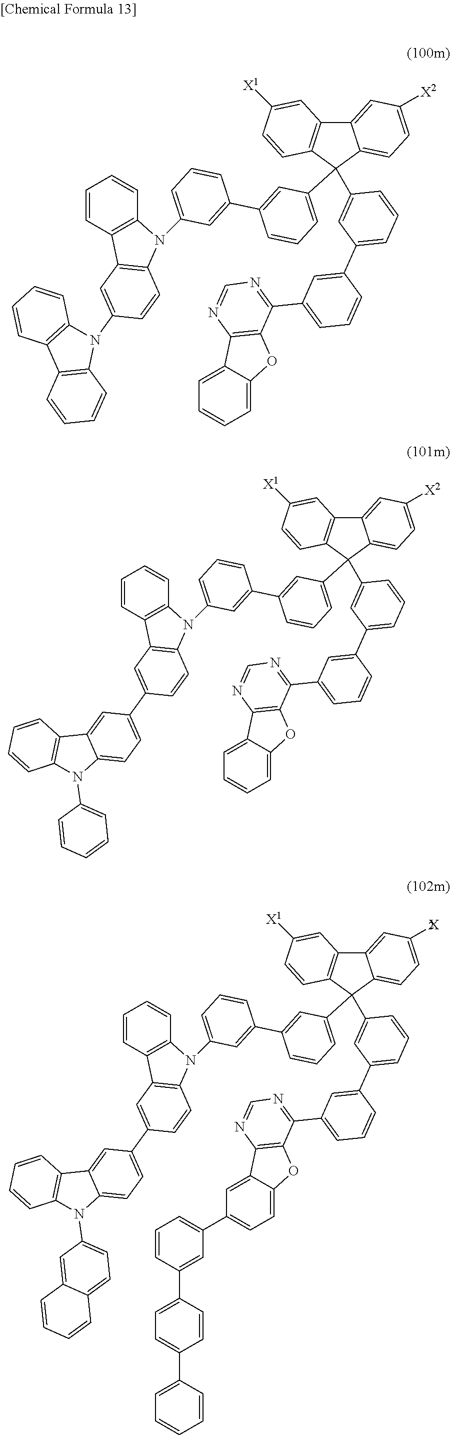

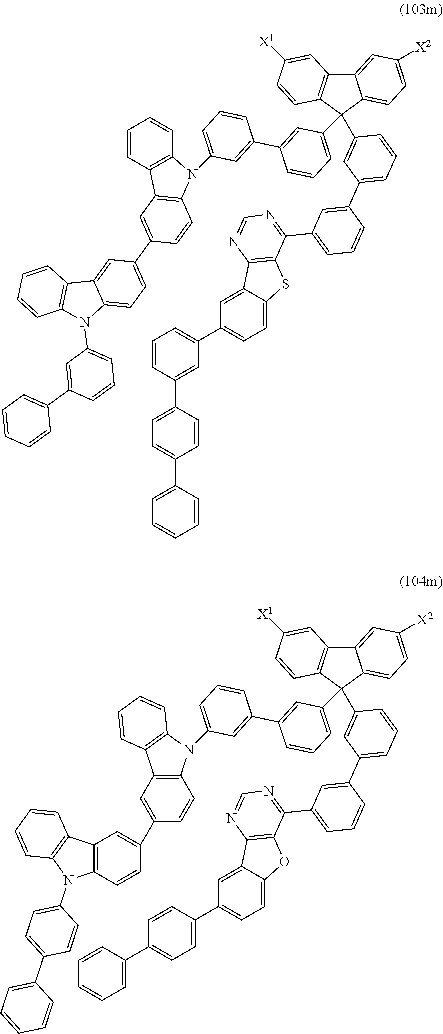

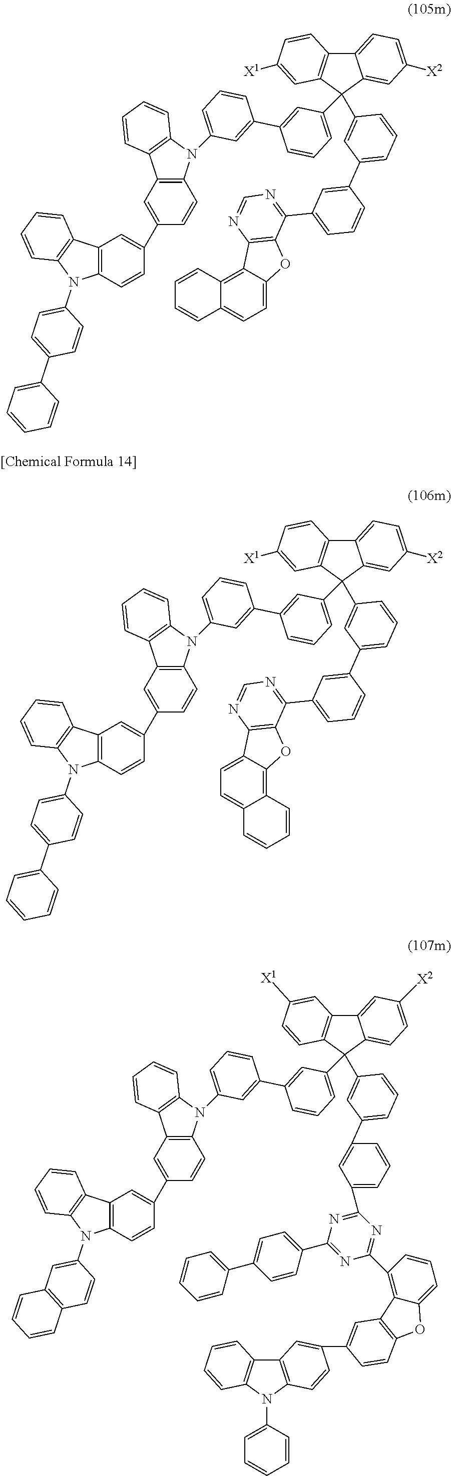

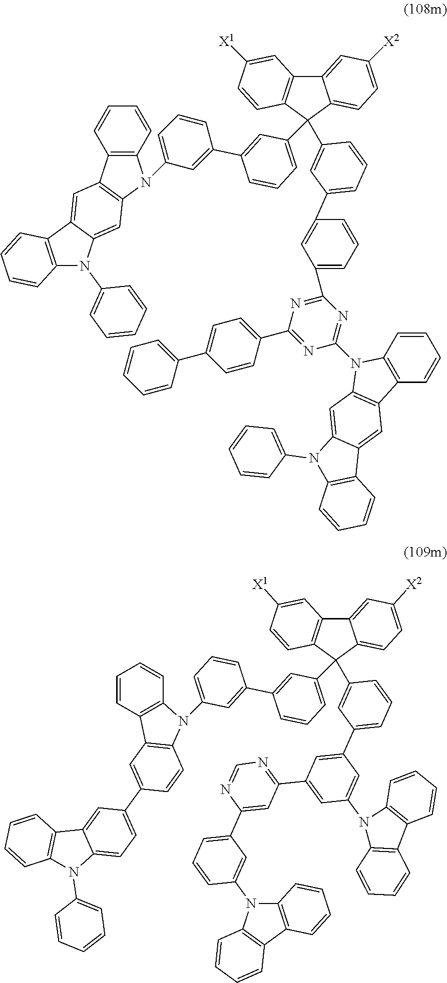

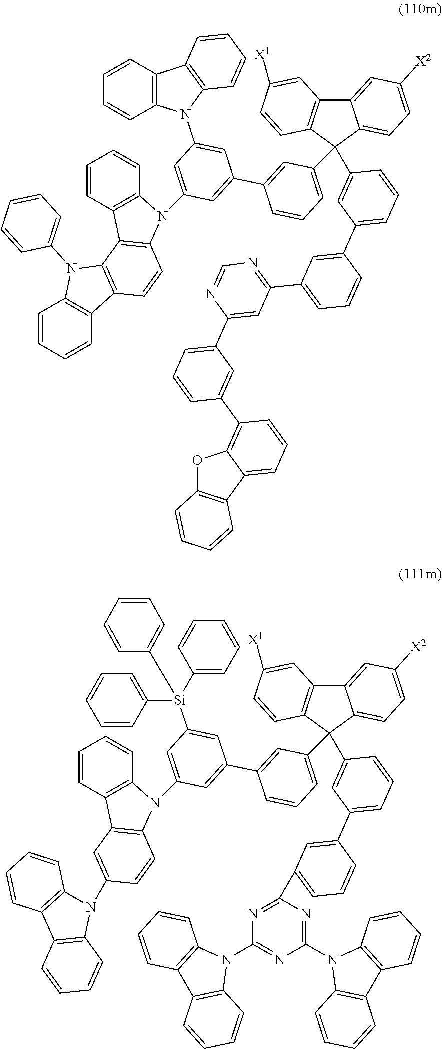

The high molecular compound of one embodiment of the present invention can be obtained in the following manner: the organic compound represented by Formula (G0) above is used as a monomer and such a monomer is polymerized by a known method utilizing Suzuki-Miyaura coupling or an Ullmann reaction, for example. In the polymerization reaction, an organic compound obtained by substituting X¹ and X² of the organic compound represented by Formula (G0) with a boronic acid, a boronic ester, a cyclic-triolborate salt, or the like may be used as a monomer. As the cyclic-triolborate salt, a lithium salt, a potassium salt, or a sodium salt may be used.

The structure described in this embodiment can be combined with any of the other embodiments as appropriate.

### Embodiment 2

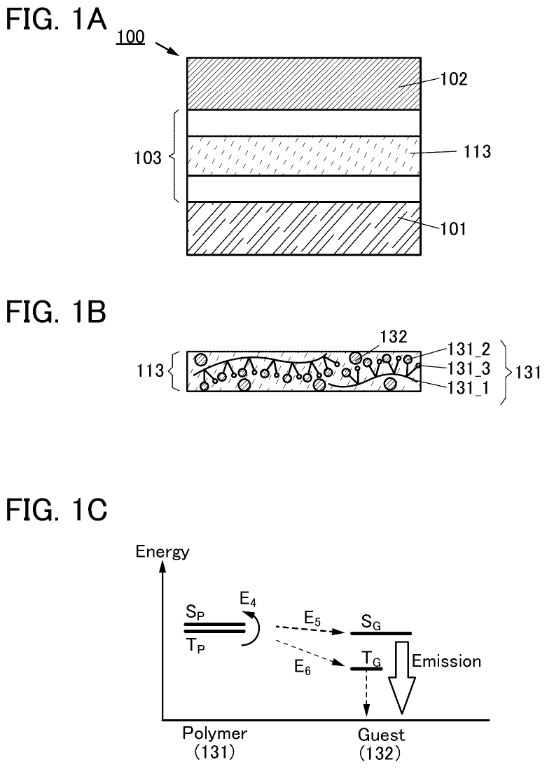

In this embodiment, a light-emitting device of one embodiment of the present invention will be described below with reference to FIGS. 1A to 1C.

### <Structure Example 1 of Light-Emitting Device>

First, a structure of the light-emitting device of one embodiment of the present invention will be described with reference to FIGS. 1A to 1C.

FIG. 1A is a schematic cross-sectional view of a light-emitting device 100 of one embodiment of the present invention.

The light-emitting device 100 includes a pair of electrodes (a first electrode 101 and a second electrode 102) and an EL layer 103 between the pair of electrodes. The EL layer 103 includes at least a light-emitting layer 113.

The EL layer 103 illustrated in FIG. 1A may include a functional layer in addition to the light-emitting layer 113. Examples of the functional layer include a hole-injection layer, a hole-transport layer, an electron-transport layer, and an electron-injection layer. Note that the functional layer may be either a single layer or stacked layers. The details of the functional layer that can be used in the light-emitting device 100 will be described in an embodiment below.

FIG. 1B is a schematic cross-sectional view illustrating an example of the light-emitting layer 113 illustrated in FIG. 1A. The light-emitting layer 113 illustrated in FIG. 1B includes a high molecular compound 131 and a guest material 132.

The high molecular compound 131 includes a main chain skeleton 131_1, a skeleton 131_2, and a skeleton 131_3 as a repeating unit. The skeleton 131_2 and the skeleton 131_3 are bonded to the main chain skeleton 131_1.

It is preferable that the skeleton 131_2 include a hole-transport skeleton and the skeleton 131_3 include an electron-transport skeleton. Alternatively, it is preferable that the skeleton 131_2 include at least one of a π-electron rich heteroaromatic skeleton and an aromatic amine skeleton and the skeleton 131_3 include a π-electron deficient heteroaromatic skeleton.

The repeating unit of the high molecular compound described in Embodiment 1 can be given as an example of the repeating unit included in the high molecular compound 131. In the case where the high molecular compound 131 includes the repeating unit of the high molecular compound described in Embodiment 1, it is possible to use a fluorenediyl group as the main chain skeleton 131_1, a skeleton in which the first arylene group and the hole-transport skeleton are bonded to each other as the skeleton 131_2, and a skeleton in which the second arylene group and the electron-transport skeleton are bonded to each other as the skeleton 131_3.

In the high molecular compound 131, the hole-transport skeleton included in the skeleton 131_2 and the electron-transport skeleton included in the skeleton 131_3 are preferably stacked by π-π interaction. This causes intramolecular charge transfer between the hole-transport skeleton included in the skeleton 131_2 and the electron-transport skeleton included in the skeleton 131_3 in the same repeating unit in the excited state. Accordingly, the efficiency of the intramolecular charge transfer in the excited state in the high molecular compound 131 can be improved.

The HOMO distribution in the repeating unit can be easily located on the hole-transport skeleton and the LUMO distribution in the repeating unit can be easily located on the electron-transport skeleton in the high molecular compound 131; thus, the intramolecular charge transfer is likely to occur between the electron-transport skeleton and the hole-transport skeleton in the same repeating unit in the excited state.

Thus, an overlap between the molecular orbital of the HOMO and the molecular orbital of the LUMO is extremely small and exchange interaction is small, leading to a small difference (ΔEₛₜ) between a singlet excitation energy level and a triplet excitation energy level in the high molecular compound 131. Accordingly, ΔEₛₜ in the high molecular compound 131 is preferably greater than 0 eV and less than or equal to 0.2 eV.

A light-emitting organic compound is used as the guest material 132, and the light-emitting organic compound is preferably a substance capable of emitting fluorescent light (hereinafter, also referred to as a fluorescent compound). A structure in which a fluorescent compound is used as the guest material 132 will be described below. The guest material 132 may be referred to as a fluorescent material or a fluorescent compound.

In the light-emitting device 100 of one embodiment of the present invention, voltage application between the pair of electrodes (the first electrode 101 and the second electrode 102) allows electrons and holes to be injected from the cathode and the anode, respectively, into the EL layer 103 and thus current flows. By recombination of the injected electrons and holes, excitons are formed. The ratio of singlet excitons to triplet excitons (hereinafter, referred to as exciton generation probability) that are generated by the recombination of the carriers (electrons and holes) is approximately 1:3 according to the statistically obtained probability. Accordingly, in a light-emitting device that contains a fluorescent material, the probability of generation of singlet excitons, which contribute to light emission, is 25% and the probability of generation of triplet excitons, which do not contribute to light emission, is 75%. Therefore, it is important to convert triplet excitons, which do not contribute to light emission, into singlet excitons, which contribute to light emission, for increasing the emission efficiency of the light-emitting device.

Thus, the high molecular compound 131 preferably has a function of generating the singlet excited state from the triplet excited state.

### <Light Emission Mechanism of Light-Emitting Device>

Next, a light emission mechanism of the light-emitting layer 113 will be described. FIG. 1C shows a correlation of energy levels of the high molecular compound 131 and the guest material 132 in the light-emitting layer 113. The following explain what terms and signs in FIG. 1C represent:
Polymer (131): the high molecular compound 131;
Guest (132): the guest material 132 (the fluorescent compound);
S_{P}: the S₁ level of the high molecular compound 131;
T_{P}: the T₁ level of the high molecular compound 131;
S_{G}: the S₁ level of the guest material 132 (the fluorescent compound); and
T_{G}: the T₁ level of the guest material 132 (the fluorescent compound).

In the light-emitting layer 113, the high molecular compound 131 is present in the largest proportion by weight, and the guest material 132 (the fluorescent material) is dispersed in the high molecular compound 131. The S₁ level of the high molecular compound 131 in the light-emitting layer 113 is preferably higher than the S₁ level of the guest material 132 (the fluorescent material). The T₁ level of the high molecular compound 131 in the light-emitting layer 113 is preferably higher than the T₁ level of the guest material 132 (the fluorescent material) in the light-emitting layer 113.

In the light-emitting device of one embodiment of the present invention, intramolecular charge transfer occurs in the excited state of the high molecular compound 131 included in the light-emitting layer 113. The S₁ level (S_{P}) and the T₁ level (T_{P}) of the high molecular compound 131 are close to each other (see FIG. 1C).

Since the S₁ level (S_{P}) and the T₁ level (T_{P}) of the high molecular compound 131 are close to each other, the high molecular compound 131 has a function of exhibiting thermally activated delayed fluorescence. In other words, the high molecular compound 131 has a function of converting triplet excitation energy into singlet excitation energy by reverse intersystem crossing (upconversion) (see Route E₄ in FIG. 1C). Thus, the triplet excitation energy of the high molecular compound 131 generated in the light-emitting layer 113 is partly converted into singlet excitation energy. In order to achieve this, an energy difference between the S₁ level (S_{P}) and the T₁ level (T_{P}) of the high molecular compound 131 is preferably greater than 0 eV and less than or equal to 0.2 eV.

The S₁ level (S_{P}) of the high molecular compound 131 is preferably higher than the S₁ level (S_{G}) of the guest material 132. This enables energy transfer from the S₁ level (S_{P}) of the high molecular compound 131 to the S₁ level (S_{G}) of the guest material 132. As a result, the guest material 132 is brought into the singlet excited state, causing light emission (see Route E₅ in FIG. 1C).

To obtain light emission efficiently from the singlet excited state of the guest material 132, the fluorescence quantum yield of the guest material 132 is preferably high, specifically, preferably 50% or higher, further preferably 70% or higher, still further preferably 90% or higher, most preferably 95% or higher.

Since direct transition from a singlet ground state to a triplet excited state in the guest material 132 is forbidden, energy transfer from the S₁ level (S_{P}) of the high molecular compound 131 to the T₁ level (T_{G}) of the guest material 132 is unlikely to be a main energy transfer process.

When transfer of the triplet excitation energy from the T₁ level (T_{P}) of the high molecular compound 131 to the T₁ level (T_{G}) of the guest material 132 occurs, the triplet excitation energy is deactivated (see Route E₆ in FIG. 1C). Thus, it is preferable that the energy transfer of Route E₆ be less likely to occur because the efficiency of generating the triplet excited state of the guest material 132 can be decreased and thermal deactivation can be suppressed. In order to achieve this, the concentration of the guest material 132 with respect to the high molecular compound 131 is preferably low; specifically, the concentration of the guest material 132 is preferably higher than or equal to 0.1 wt % and lower than or equal to 5 wt %, further preferably higher than or equal to 0.1 wt % and lower than or equal to 3 wt %, still further preferably higher than or equal to 0.1 wt % and lower than or equal to 1 wt %.

Note that when the direct carrier recombination process is dominant in the guest material 132, a large number of triplet excitons of the guest material are generated in the light-emitting layer 113, resulting in decreased emission efficiency due to thermal deactivation. Thus, it is preferable that the probability of the process in which carriers are recombined in the high molecular compound 131 and energy is transferred to the guest material (Routes E₄ and E₅ in FIG. 1C) be higher than the probability of the direct carrier recombination process in the guest material 132 because the efficiency of generating the triplet excited state of the guest material 132 can be decreased and thermal deactivation can be suppressed. In order to achieve this, the concentration of the guest material 132 with respect to the high molecular compound 131 is preferably low; specifically, the concentration of the guest material 132 is preferably higher than or equal to 0.1 wt % and lower than or equal to 5 wt %, further preferably higher than or equal to 0.1 wt % and lower than or equal to 3 wt %, still further preferably higher than or equal to 0.1 wt % and lower than or equal to 1 wt %.

By making all the energy transfer processes of Routes E₄ and E₅ efficiently occur in the above-described manner, both the singlet excitation energy and the triplet excitation energy of the high molecular compound 131 can be efficiently converted into the singlet excited state of the guest material 132, whereby the light-emitting device 100 can emit light with high emission efficiency.

When the light-emitting layer 113 has the above-described structure, light emission from the guest material 132 of the light-emitting layer 113 can be obtained efficiently.

Thus, in one embodiment of the present invention, it is preferable that, in the high molecular compound 131, the main chain skeleton 131_1 having a hole-transport property and the skeleton 131_2 having an electron-transport property be bonded or polymerized to each other through the skeleton 131_3. Alternatively, it is preferable that, in the high molecular compound 131, the π-electron deficient heteroaromatic skeleton and at least one of the π-electron rich heteroaromatic skeleton and the aromatic amine skeleton be bonded or polymerized to each other through the skeleton 131_3. Note that the details of the skeleton 131_3 will be described later.

### <Energy Transfer Mechanism>

Next, factors controlling the processes of intermolecular energy transfer between the high molecular compound 131 and the guest material 132 will be described. As mechanisms of the intermolecular energy transfer, two mechanisms, i.e., Förster mechanism (dipole-dipole interaction) and Dexter mechanism (electron exchange interaction), have been proposed.

### <<Förster Mechanism>>

In Förster mechanism, energy transfer does not require direct contact between molecules and energy is transferred through a resonant phenomenon of dipolar oscillation between the high molecular compound 131 and the guest material 132. By the resonant phenomenon of dipolar oscillation, the high molecular compound 131 provides energy to the guest material 132; thus, the high molecular compound 131 in an excited state is brought to a ground state and the guest material 132 in a ground state is brought to an excited state. Note that the rate constant k_{h*→g} of Förster mechanism is expressed by Formula (1).

$$\begin{matrix}[ Formula 1 ] & \\ {k}_{{h}^{*} \to g} = \frac{9000 {K}^{2} ϕln10}{128 {π}^{5} {n}^{4} N τ {R}^{6}} \int \frac{{f}_{h}^{′} ( ν ) {ε}_{g} ( ν )}{{ν}^{4}} d ν & ( 1 )\end{matrix}$$

In Formula (1), ν denotes a frequency, f′ₕ(ν) denotes a normalized emission spectrum of the high molecular compound 131 (a fluorescent spectrum in energy transfer from a singlet excited state, and a phosphorescent spectrum in energy transfer from a triplet excited state), ε_{g}(ν) denotes a molar absorption coefficient of the guest material 132, N denotes Avogadro's number, n denotes a refractive index of a medium, R denotes an intermolecular distance between the high molecular compound 131 and the guest material 132, τ denotes a measured lifetime of an excited state (fluorescence lifetime or phosphorescence lifetime), ϕ denotes an emission quantum yield (a fluorescence quantum yield in energy transfer from a singlet excited state, and a phosphorescence quantum yield in energy transfer from a triplet excited state), and K² denotes a coefficient (0 to 4) of orientation of a transition dipole moment between the high molecular compound 131 and the guest material 132. Note that K² is ⅔ in random orientation.

### <<Dexter Mechanism>>

In Dexter mechanism, the high molecular compound 131 and the guest material 132 are close to a contact effective range where their orbitals overlap with each other, and the high molecular compound 131 in an excited state and the guest material 132 in a ground state exchange their electrons, which leads to energy transfer. Note that the rate constant k_{h*→g} of Dexter mechanism is expressed by Formula (2).

$$\begin{matrix}[ Formula 2 ] & \\ {k}_{{h}^{*} \to g} = ( \frac{2 π}{h} ) {K}^{2} exp ( - \frac{2 R}{L} ) \int {f}_{h}^{′} ( ν ) {ε}_{g}^{′} ν ( d ν ) & ( 2 )\end{matrix}$$

In Formula (2), h denotes a Planck constant, K denotes a constant having an energy dimension, ν denotes a frequency, f′ₕ(ν) denotes a normalized emission spectrum of the high molecular compound 131 (a fluorescent spectrum in energy transfer from a singlet excited state, and a phosphorescent spectrum in energy transfer from a triplet excited state), ε′_{g}(ν) denotes a normalized absorption spectrum of the guest material 132, L denotes an effective molecular radius, and R denotes an intermolecular distance between the high molecular compound 131 and the guest material 132.

Here, the efficiency of energy transfer from the high molecular compound 131 to the guest material 132 (energy transfer efficiency ϕ_{ET}) is expressed by Formula (3). In the formula, kᵣ denotes a rate constant of a light-emission process (fluorescence in energy transfer from a singlet excited state, and phosphorescence in energy transfer from a triplet excited state) of the high molecular compound 131, kₙ denotes a rate constant of a non-light-emission process (thermal deactivation or intersystem crossing) of the high molecular compound 131, and τ denotes a measured lifetime of an excited state of the high molecular compound 131.

$$\begin{matrix}[ Formula 3 ] & \\ {ϕ}_{E T} = \frac{{k}_{{h}^{*} \to g}}{{k}_{r} + {k}_{n} + {k}_{{h}^{*} \to g}} = \frac{{k}_{{h}^{*} \to g}}{( \frac{1}{τ} ) + {k}_{{h}^{*} \to g}} & ( 3 )\end{matrix}$$

According to Formula (3), it is found that the energy transfer efficiency ϕ_{ET} can be increased by increasing the rate constant k_{h*→g} of energy transfer so that another competing rate constant kᵣ+kₙ (=1/τ) becomes relatively small.

### <<Concept for Promoting Energy Transfer>>

First, energy transfer by Förster mechanism is considered. When Formula (1) is substituted into Formula (3), τ can be eliminated. Thus, in Förster mechanism, the energy transfer efficiency ϕ_{ET} does not depend on the lifetime τ of the excited state of the high molecular compound 131. In addition, it can be said that the energy transfer efficiency ϕ_{ET} is higher when the quantum yield ϕ (here, the fluorescence quantum efficiency because energy transfer from a singlet excited state is discussed) is higher. In general, the emission quantum yield of an organic compound in a triplet excited state is extremely low at room temperature. Thus, in the case where the high molecular compound 131 is in a triplet excited state, a process of energy transfer by Förster mechanism can be ignored, and a process of energy transfer by Förster mechanism is considered only in the case where the high molecular compound 131 is in a singlet excited state.

Furthermore, it is preferable that the emission spectrum (the fluorescent spectrum in the case where energy transfer from a singlet excited state is discussed) of the high molecular compound 131 largely overlap with the absorption spectrum (absorption corresponding to the transition from the singlet ground state to the singlet excited state) of the guest material 132. Moreover, it is preferable that the molar absorption coefficient of the guest material 132 be also high. This means that the emission spectrum of the high molecular compound 131 preferably overlaps largely with the absorption band of the guest material 132 that is on the longest wavelength side. Since direct transition from the singlet ground state to the triplet excited state of the guest material 132 is forbidden, the molar absorption coefficient of the guest material 132 in the triplet excited state can be ignored. Thus, a process of energy transfer to a triplet excited state of the guest material 132 by Förster mechanism can be ignored, and only a process of energy transfer to a singlet excited state of the guest material 132 is considered. That is, in Förster mechanism, a process of energy transfer from the singlet excited state of the high molecular compound 131 to the singlet excited state of the guest material 132 is considered.

Next, energy transfer by Dexter mechanism is considered. According to Formula (2), in order to increase the rate constant k_{h*→g}, it is preferable that the emission spectrum (a fluorescent spectrum in the case where energy transfer from the singlet excited state is discussed) of the high molecular compound 131 largely overlap with the absorption spectrum (absorption corresponding to the transition from the singlet ground state to the singlet excited state) of the guest material 132. Therefore, the energy transfer efficiency can be optimized by making the emission spectrum of the high molecular compound 131 overlap with the absorption band of the guest material 132 that is on the longest wavelength side.

When Formula (2) is substituted into Formula (3), it is found that the energy transfer efficiency ϕ_{ET} in Dexter mechanism depends on t. In Dexter mechanism, which is a process of energy transfer based on the electron exchange, as well as the energy transfer from the singlet excited state of the high molecular compound 131 to the singlet excited state of the guest material 132, energy transfer from the triplet excited state of the high molecular compound 131 to the triplet excited state of the guest material 132 occurs.

In the light-emitting device of one embodiment of the present invention in which the guest material 132 is a fluorescent material, the efficiency of energy transfer to the triplet excited state of the guest material 132 is preferably low. That is, the energy transfer efficiency based on Dexter mechanism from the high molecular compound 131 to the guest material 132 is preferably low and the energy transfer efficiency based on Förster mechanism from the high molecular compound 131 to the guest material 132 is preferably high.

As described above, the energy transfer efficiency in Förster mechanism does not depend on the lifetime of the excited state of the high molecular compound 131. By contrast, the energy transfer efficiency in Dexter mechanism depends on the excitation lifetime of the high molecular compound 131. Thus, to reduce the energy transfer efficiency in Dexter mechanism, the excitation lifetime ti of the high molecular compound 131 is preferably short.

In view of this, one embodiment of the present invention provides a light-emitting device including the high molecular compound 131 functioning as an energy donor (host material) capable of efficiently transferring energy to the guest material 132. The high molecular compound 131 has a feature that a singlet excitation energy level and a triplet excitation energy level are close to each other. Accordingly, transition from a triplet exciton generated in the light-emitting layer 113 to a singlet exciton (reverse intersystem crossing) is likely to occur. This can increase the efficiency of generating singlet excitons in the light-emitting layer 113. Furthermore, in order to facilitate energy transfer from the singlet excited state of the high molecular compound 131 to the singlet excited state of the guest material 132 functioning as an energy acceptor, it is preferable that the emission spectrum of the high molecular compound 131 overlap with the absorption band of the guest material 132 that is on the longest wavelength side (lowest energy side). Thus, the efficiency of generating the singlet excited state of the guest material 132 can be increased.

In addition, a fluorescence lifetime of a thermally activated delayed fluorescence component in light emitted from the high molecular compound 131 is preferably short, specifically, preferably 10 ns or longer and 50 μs or shorter, further preferably 10 ns or longer and 30 μs or shorter.

The proportion of a thermally activated delayed fluorescence component in the light emitted from the high molecular compound 131 is preferably high. Specifically, the proportion of a thermally activated delayed fluorescence component in the light emitted from the high molecular compound 131 is preferably higher than or equal to 5%, further preferably higher than or equal to 10%.

### <Material>

Next, components of a light-emitting device of one embodiment of the present invention will be described in detail.

### <<Light-Emitting Layer>>

Materials that can be used for the light-emitting layer 113 will be described below.

The high molecular compound 131 includes the main chain skeleton 131_1, the skeleton 131_2, and the skeleton 131_3 as the repeating unit, as described above. The skeleton 131_2 and the skeleton 131_3 are bonded to the main chain skeleton 131_1.

It is preferable that the skeleton 131_2 include a hole-transport skeleton and the skeleton 131_3 include an electron-transport skeleton. Alternatively, it is preferable that the skeleton 131_2 include at least one of a π-electron rich heteroaromatic skeleton and an aromatic amine skeleton and the skeleton 131_3 include a π-electron deficient heteroaromatic skeleton.

The repeating unit of the high molecular compound described in Embodiment 1 can be given as an example of the repeating unit included in the high molecular compound 131. In the case where the high molecular compound 131 includes the repeating unit of the high molecular compound described in Embodiment 1, it is possible to use a fluorenediyl group as the main chain skeleton 131_1, a skeleton in which the first arylene group and the hole-transport skeleton are bonded to each other as the skeleton 131_2, and a skeleton in which the second arylene group and the electron-transport skeleton are bonded to each other as the skeleton 131_3.

In the light-emitting layer 113, although the guest material 132 is not particularly limited, examples of the guest material 132 include a pyrene derivative, an anthracene derivative, a triphenylene derivative, a fluorene derivative, a carbazole derivative, a dibenzothiophene derivative, a dibenzofuran derivative, a dibenzoquinoxaline derivative, a quinoxaline derivative, a pyridine derivative, a pyrimidine derivative, a phenanthrene derivative, and a naphthalene derivative.

A pyrene derivative is particularly preferable because it has a high emission quantum yield. Specific examples of the pyrene derivative include N,N-bis(3-methylphenyl)-N,N-bis[3-(9-phenyl-9H-fluoren-9-yl)phenyl]pyrene-1,6-diamine (abbreviation: 1,6mMemFLPAPrn), N,N-diphenyl-N,N-bis[4-(9-phenyl-9H-fluoren-9-yl)phenyl]pyrene-1,6-diamine (abbreviation: 1,6FLPAPrn), N,N′-bis(dibenzofuran-2-yl)-N,N-diphenylpyrene-1,6-diamine (abbreviation: 1,6FrAPrn), N,N-bis(dibenzothiophen-2-yl)-N,N-diphenylpyrene-1,6-diamine (abbreviation: 1,6ThAPrn), N,N-(pyrene-1,6-diyl)bis[(N-phenylbenzo[b]naphtho[1,2-d]furan)-6-amine] (abbreviation: 1,6BnfAPrn), N,N-(pyrene-1,6-diyl)bis[(N-phenylbenzo[b]naphtho[1,2-d]furan)-8-amine](abbreviation: 1,6BnfAPrn-02), and N,N-(pyrene-1,6-diyl)bis[(6,N-diphenylbenzo[b]naphtho[1,2-d]furan)-8-amine] (abbreviation: 1,6BnfAPrn-03).

In addition, it is possible to use, as the guest material 132, 5,6-bis[4-(10-phenyl-9-anthryl)phenyl]-2,2′-bipyridine (abbreviation: PAP2BPy), 5,6-bis[4′-(10-phenyl-9-anthryl)biphenyl-4-yl]-2,2′-bipyridine (abbreviation: PAPP2BPy), N,N-bis[4-(9H-carbazol-9-yl)phenyl]-N,N-diphenylstilbene-4,4′-diamine (abbreviation: YGA2S), 4-(9H-carbazol-9-yl)-4′-(10-phenyl-9-anthryl)triphenylamine (abbreviation: YGAPA), 4-(9H-carbazol-9-yl)-4′-(9,10-diphenyl-2-anthryl)triphenylamine (abbreviation: 2YGAPPA), N,9-diphenyl-N-[4-(10-phenyl-9-anthryl)phenyl]-9H-carbazol-3-amine (abbreviation: PCAPA), 4-(10-phenyl-9-anthryl)-4′-(9-phenyl-9H-carbazol-3-yl)triphenylamine (abbreviation: PCBAPA), 4-[4-(10-phenyl-9-anthryl)phenyl]-4′-(9-phenyl-9H-carbazol-3-yl)triphenylamine (abbreviation: PCBAPBA), perylene, 2,5,8,11-tetra-tert-butylperylene (abbreviation: TBP), N,N′-(2-tert-butylanthracene-9,10-diyldi-4,1-phenylene)bis(N,N,N-triphenyl-1,4-phenylenediamine) (abbreviation: DPABPA), N,9-diphenyl-N-[4-(9,10-diphenyl-2-anthryl)phenyl]-9H-carbazol-3-amine (abbreviation: 2PCAPPA), N-[4-(9,10-diphenyl-2-anthryl)phenyl]-N,N,N-triphenyl-1,4-phenylenediamine (abbreviation: 2DPAPPA), or the like.

It is also possible to use, as the guest material 132, N-[9,10-bis(biphenyl-2-yl)-2-anthryl]-N,9-diphenyl-9H-carbazol-3-amine (abbreviation: 2PCABPhA), N-(9,10-diphenyl-2-anthryl)-N,N,N-triphenyl-1,4-phenylenediamine (abbreviation: 2DPAPA), N-[9,10-bis(biphenyl-2-yl)-2-anthryl]-N,N,N-triphenyl-1,4-phenylenediamine (abbreviation: 2DPABPhA), 9,10-bis(biphenyl-2-yl)-N-[4-(9H-carbazol-9-yl)phenyl]-N-phenylanthracen-2-amine (abbreviation: 2YGABPhA), N,N,9-triphenylanthracen-9-amine (abbreviation: DPhAPhA), coumarin 545T, N,N-diphenylquinacridone (abbreviation: DPQd), rubrene, 5,12-bis(biphenyl-4-yl)-6,11-diphenyltetracene (abbreviation: BPT), 2-(2-{2-[4-(dimethylamino)phenyl]ethenyl}-6-methyl-4H-pyran-4-ylidene)propanedinitrile (abbreviation: DCM1), 2-{2-methyl-6-[2-(2,3,6,7-tetrahydro-1H,5H-benzo[ij]quinolizin-9-yl)ethenyl]-4H-pyran-4-ylidene}propanedinitrile (abbreviation: DCM2), N,N,N,N-tetrakis(4-methylphenyl)tetracene-5,11-diamine (abbreviation: p-mPhTD), 7,14-diphenyl-N,N,N,N-tetrakis(4-methylphenyl)acenaphtho[1,2-a]fluoranthene-3,10-diamine (abbreviation: p-mPhAFD), 2-{2-isopropyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTI), 2-{2-tert-butyl-6-[2-(1,1,7,7-tetramethyl-2,3,6,7-tetrahydro-1H,5H-benzo[ij]quinolizin-9-yl)ethenyl]-4H-pyran-4-ylidene}propanedinitrile (abbreviation: DCJTB), 2-(2,6-bis{2-[4-(dimethylamino)phenyl]ethenyl}-4H-pyran-4-ylidene)propanedinitrile (abbreviation: BisDCM), 2-{2,6-bis[2-(8-methoxy-1,1,7,7-tetramethyl-2,3,6,7-tetrahydro-1H,5H-benzo[ij]quinolizin-9-yl)ethenyl]-4H-pyran-4-ylidene}propanedinitrile (abbreviation: BisDCJ™), 1,6BnfAPrn-03, 3,10-bis[N-(9-phenyl-9H-carbazol-2-yl)-N-phenylamino]naphtho[2,3-b;6,7-b′]bisbenzofuran (abbreviation: 3,10PCA2Nbf(IV)-02), 3,10-bis[N-(dibenzofuran-3-yl)-N-phenylamino]naphtho[2,3-b;6,7-b′]bisbenzofuran (abbreviation: 3,10FrA2Nbf(IV)-02), or the like. In particular, pyrenediamine compounds such as 1,6FLPAPrn, 1,6mMemFLPAPrn, and 1,6BnfAPrn-03 can be used, for example.

The light-emitting layer 113 may contain a different material in addition to the high molecular compound 131 and the guest material 132. For example, a material described in an embodiment below as a hole-transport material that can be used for a hole-transport layer in a light-emitting device can be used as the different material. Moreover, a material described in an embodiment below as an electron-transport material that can be used for an electron-transport layer can be used as the different material.

As the material that can be used for the light-emitting layer 113, a material capable of being dissolved in a solvent that can dissolve the high molecular compound of one embodiment of the present invention is preferable.

Note that the light-emitting layer 113 can be formed by an evaporation method (including a vacuum evaporation method), an inkjet method, a coating method, a nozzle-printing method, gravure printing, or the like. Besides the above-mentioned materials, an inorganic compound such as a quantum dot or a high molecular compound (e.g., an oligomer, a dendrimer, and a polymer) may be used.

The structure described in this embodiment can be combined with any of the other embodiments as appropriate.

### Embodiment 3

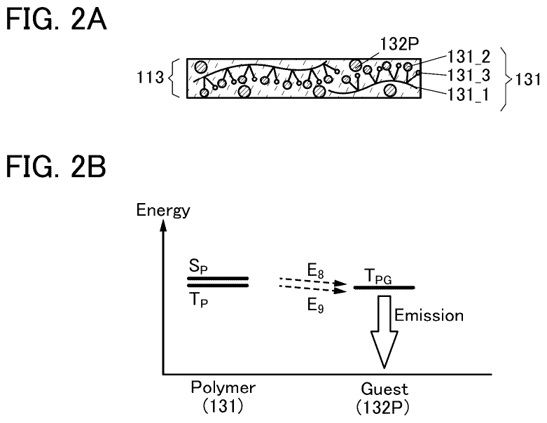

In this embodiment, a light-emitting device having a structure different from that described in Embodiment 2 and a light emission mechanism of the light-emitting device will be described with reference to FIGS. 2A and 2B.

### <Structure Example 2 of Light-Emitting Device>

A light-emitting device having a structure in which the guest material 132 in the light-emitting device 100 described in Embodiment 2 is replaced with a guest material 132P, which is a substance that can emit phosphorescent light, and a light emission mechanism of the light-emitting device will be described.

FIG. 2A is a schematic cross-sectional view illustrating an example of the light-emitting layer 113 that can be used in this structure. The light-emitting layer 113 illustrated in FIG. 2A includes the high molecular compound 131 and the guest material 132P.

The high molecular compound 131 described in Embodiment 2 can also be used in this structure example.

A light-emitting organic compound is used as the guest material 132P, and the light-emitting organic compound is preferably a substance capable of emitting phosphorescent light (hereinafter, also referred to as a phosphorescent compound). A structure in which a phosphorescent compound is used as the guest material 132P will be described below. The guest material 132P may be referred to as a phosphorescent material or a phosphorescent compound.

### <Light Emission Mechanism of Light-Emitting Device>

Next, alight emission mechanism of the light-emitting layer 113 in this structure example will be described.

FIG. 2B shows a correlation of energy levels of the high molecular compound 131 and the guest material 132P in the light-emitting layer 113. The following explain what terms and signs in FIG. 2B represent:
Polymer (131): the high molecular compound 131;
Guest (132P): the guest material 132P (the phosphorescent material);
S_{P}: the S₁ level of the high molecular compound 131;
T_{P}: the T₁ level of the high molecular compound 131; and
T_{PG}: the T₁ level of the guest material 132P (the phosphorescent material).

In the light-emitting layer 113, the high molecular compound 131 is present in the largest proportion by weight, and the guest material 132P (the phosphorescent material) is dispersed in the high molecular compound 131. The T₁ level of the high molecular compound 131 in the light-emitting layer 113 is preferably higher than the T₁ level of the guest material (the guest material 132P) in the light-emitting layer 113.

In the light-emitting device of one embodiment of the present invention, intramolecular charge transfer occurs in the excited state of the high molecular compound 131 included in the light-emitting layer 113. The S₁ level (S_{P}) and the T₁ level (T_{P}) of the high molecular compound 131 are close to each other (see FIG. 2B).

Since the S₁ level (S_{P}) and the T₁ level (T_{P}) of the high molecular compound 131 are close to each other, both energies of S_{P} and T_{P} are transferred to the lowest level of the triplet excited state of the guest material 132P (the phosphorescent material), so that light emission is obtained (see Routes E₈ and E₉ in FIG. 2B).

The T₁ level (T_{P}) of the high molecular compound 131 is preferably higher than the T₁ level (T_{PG}) of the guest material 132P. This enables energy transfer from the S₁ level (S_{P}) and the T₁ level (T_{P}) of the high molecular compound 131 to the T₁ level (T_{PG}) of the guest material 132P.

When the light-emitting layer 113 has the above-described structure, light emission from the guest material 132P (the phosphorescent material) of the light-emitting layer 113 can be obtained efficiently.

The mechanism of the energy transfer process between the molecules of the high molecular compound 131 and the guest material 132P can be described using two mechanisms, i.e., Förster mechanism (dipole-dipole interaction) and Dexter mechanism (electron exchange interaction), as in Embodiment 2. For Förster mechanism and Dexter mechanism, refer to Embodiment 2.

### <<Concept for Promoting Energy Transfer>>

In energy transfer by Förster mechanism, the energy transfer efficiency ϕ_{ET} is higher when the quantum yield 4 (the fluorescence quantum efficiency when energy transfer from a singlet excited state is discussed) is higher. Furthermore, it is preferable that the emission spectrum (the fluorescent spectrum in the case where energy transfer from a singlet excited state is discussed) of the high molecular compound 131 largely overlap with the absorption spectrum (absorption corresponding to the transition from the singlet ground state to the triplet excited state) of the guest material 132P. Moreover, it is preferable that the molar absorption coefficient of the guest material 132P be also high. This means that the emission spectrum of the high molecular compound 131 preferably overlaps largely with the absorption band of the guest material 132P that is on the longest wavelength side.

In energy transfer by Dexter mechanism, in order to increase the rate constant k_{h*→g}, it is preferable that the emission spectrum (a fluorescent spectrum in the case where energy transfer from a singlet excited state is discussed) of the high molecular compound 131 largely overlap with the absorption spectrum (absorption corresponding to the transition from the singlet ground state to the triplet excited state) of the guest material 132P. Therefore, the energy transfer efficiency can be optimized by making the emission spectrum of the high molecular compound 131 overlap with the absorption band of the guest material 132P that is on the longest wavelength side.

In view of the above, one embodiment of the present invention provides a light-emitting device including the high molecular compound 131 functioning as an energy donor (host material) capable of efficiently transferring energy to the guest material 132P. The high molecular compound 131 has a feature that the singlet excitation energy level and the triplet excitation energy level are close to each other. Thus, the excited state of the high molecular compound 131 in the light-emitting layer 113 can be formed with low energy. Accordingly, the driving voltage of the light-emitting device 100 can be reduced. Furthermore, in order to facilitate energy transfer from the singlet excited state of the high molecular compound 131 to the triplet excited state of the guest material 132P functioning as an energy acceptor, it is preferable that the emission spectrum of the high molecular compound 131 overlap with the absorption band of the guest material 132P that is on the longest wavelength side (lowest energy side). Thus, the efficiency of generating the triplet excited state of the guest material 132P can be increased.

<Example of Material that can be Used in Light-Emitting Layer>

Next, materials that can be used in the light-emitting layer 113 will be described below.

The high molecular compound 131 described in Embodiment 2 can also be used in this structure example.

The guest material 132P (the phosphorescent material) preferably contains a metal element with large spin-orbit interaction, and can be an organometallic complex, a metal complex, or a rare earth metal complex, for example. Specifically, the guest material 132P preferably contains a transition metal element. It is particularly preferable that the guest material 132P contain a platinum group element (ruthenium (Ru), rhodium (Rh), palladium (Pd), osmium (Os), iridium (Ir), or platinum (Pt)), especially iridium, in which case the probability of direct transition between the singlet ground state and the triplet excited state can be increased.

<<Phosphorescent Substance (from 450 nm to 570 nm, Blue or Green)>>

As examples of a phosphorescent substance which emits blue or green light and whose emission spectrum has a peak wavelength of greater than or equal to 450 nm and less than or equal to 570 nm, the following substances can be given.

Examples include organometallic complexes having a 4H-triazole ring, such as tris{2-[5-(2-methylphenyl)-4-(2,6-dimethylphenyl)-4H-1,2,4-triazol-3-yl-κN²]phenyl-κC}iridium(III) (abbreviation: [Ir(mpptz-dmp)₃]), tris(5-methyl-3,4-diphenyl-4H-1,2,4-triazolato)iridium(III) (abbreviation: [Ir(Mptz)₃]), tris[4-(3-biphenyl)-5-isopropyl-3-phenyl-4H-1,2,4-triazolato]iridium(III) (abbreviation: [Ir(iPrptz-3b)₃]), and tris[3-(5-biphenyl)-5-isopropyl-4-phenyl-4H-1,2,4-triazolato]iridium(III) (abbreviation: [Ir(iPr5btz)₃]); organometallic complexes having a 1H-triazole ring, such as tris[3-methyl-1-(2-methylphenyl)-5-phenyl-1H-1,2,4-triazolato]iridium(III) (abbreviation: [Ir(Mptz1-mp)₃]) and tris(1-methyl-5-phenyl-3-propyl-1H-1,2,4-triazolato)iridium(III) (abbreviation: [Ir(Prptz1-Me)₃]); organometallic complexes having an imidazole ring, such as fac-tris[1-(2,6-diisopropylphenyl)-2-phenyl-1H-imidazole]iridium(III) (abbreviation: [Ir(iPrpim)₃]), tris[3-(2,6-dimethylphenyl)-7-methylimidazo[1,2-f]phenanthridinato]iridium(III) (abbreviation: [Ir(dmpimpt-Me)₃]), and tris(2-[1-{2,6-bis(1-methylethyl)phenyl}-1H-imidazol-2-yl-κN³]-4-cyanophenyl-κC)iridium(III) (abbreviation: CNImIr); organometallic complexes having a benzimidazolidene ring, such as tris[(6-tert-butyl-3-phenyl-2H-imidazo[4,5-b]pyrazin-1-yl-κC²)phenyl-κC]iridium(III) (abbreviation: [Ir(cb)₃]) and [2-({5-[3-(3,5-di-tert-butylphenyl)benzimidazol-1-yl-2-ylidene-κC²]-4′-cyano-2′,6′-dimethylbiphenyl-3-yl-κC⁴}oxy)-9-(4-tert-butyl-2-pyridinyl-κN)carbazole-2,1-diyl-κC]platinum(II) (abbreviation: Pt(mmtBubdmCPOcztBupy)); and organometallic complexes in which a phenylpyridine derivative having an electron-withdrawing group is a ligand, such as bis[2-(4′,6′-difluorophenyl)pyridinato-N,C^{2′}]iridium(III) tetrakis(1-pyrazolyl)borate (abbreviation: FIr6), bis[2-(4′,6′-difluorophenyl)pyridinato-N,C^{2′}]iridium(III) picolinate (abbreviation: FIrpic), bis{2-[3′,5′-bis(trifluoromethyl)phenyl]pyridinato-N,C^{2′}}iridium(III) picolinate (abbreviation: [Ir(CF₃ppy)₂(pic)]), and bis[2-(4′,6′-difluorophenyl)pyridinato-N,C^{2′}]iridium(III) acetylacetonate (abbreviation: FIr(acac)).

<<Phosphorescent Substance (from 495 nm to 590 nm, Green or Yellow)>>

As examples of a phosphorescent substance which emits green or yellow light and whose emission spectrum has a peak wavelength of greater than or equal to 495 nm and less than or equal to 590 nm, the following substances can be given.

Examples include organometallic iridium complexes having a pyrimidine ring, such as tris(4-methyl-6-phenylpyrimidinato)iridium(III) (abbreviation: [Ir(mppm)₃]), tris(4-t-butyl-6-phenylpyrimidinato)iridium(III) (abbreviation: [Ir(tBuppm)₃]), (acetylacetonato)bis(6-methyl-4-phenylpyrimidinato)iridium(III) (abbreviation: [Ir(mppm)₂(acac)]), (acetylacetonato)bis(6-tert-butyl-4-phenylpyrimidinato)iridium(III) (abbreviation: [Ir(tBuppm)₂(acac)]), (acetylacetonato)bis[6-(2-norbornyl)-4-phenylpyrimidinato]iridium(III) (abbreviation: [Ir(nbppm)₂(acac)]), (acetylacetonato)bis[5-methyl-6-(2-methylphenyl)-4-phenylpyrimidinato]iridium(III) (abbreviation: [Ir(mpmppm)₂(acac)]), (acetylacetonato)bis{4,6-dimethyl-2-[6-(2,6-dimethylphenyl)-4-pyrimidinyl-κN³]phenyl-κC}iridium(III) (abbreviation: [Ir(dmppm-dmp)₂(acac)]), and (acetylacetonato)bis(4,6-diphenylpyrimidinato)iridium(III) (abbreviation: [Ir(dppm)₂(acac)]); organometallic iridium complexes having a pyrazine ring, such as (acetylacetonato)bis(3,5-dimethyl-2-phenylpyrazinato)iridium(III) (abbreviation: [Ir(mppr-Me)₂(acac)]) and (acetylacetonato)bis(5-isopropyl-3-methyl-2-phenylpyrazinato)iridium(III) (abbreviation: [Ir(mppr-iPr)₂(acac)]); organometallic iridium complexes having a pyridine ring, such as tris(2-phenylpyridinato-N,C^{2′})iridium(III) (abbreviation: [Ir(ppy)₃]), bis(2-phenylpyridinato-N,C^{2′})iridium(III) acetylacetonate (abbreviation: [Ir(ppy)₂(acac)]), bis(benzo[h]quinolinato)iridium(III) acetylacetonate (abbreviation: [Ir(bzq)₂(acac)]), tris(benzo[h]quinolinato)iridium(III) (abbreviation: [Ir(bzq)₃]), tris(2-phenylquinolinato-N,C^{2′})iridium(III) (abbreviation: [Ir(pq)₃]), bis(2-phenylquinolinato-N,C^{2′})iridium(III) acetylacetonate (abbreviation: [Ir(pq)₂(acac)]), bis[2-(2-pyridinyl-κN)phenyl-κC][2-(4-phenyl-2-pyridinyl-κN)phenyl-κC]iridium(III) (abbreviation: [Ir(ppy)₂(4dppy)]), bis[2-(2-pyridinyl-κN)phenyl-κC][2-(4-methyl-5-phenyl-2-pyridinyl-κN)phenyl-κC], [2-d₃-methyl-8-(2-pyridinyl-κN)benzofuro[2,3-b]pyridine-κC]bis[2-(5-d₃-methyl-2-pyridinyl-κN²)phenyl-κC]iridium(III) (abbreviation: Ir(5mppy-d₃)₂(mbfpypy-d₃)), {2-(methyl-d₃)-8-[4-(1-methylethyl-1-d)-2-pyridinyl-κN]benzofuro[2,3-b]pyridin-7-yl-κC}bis{5-(methyl-d₃)-2-[5-(methyl-d₃)-2-pyridinyl-κN]phenyl-κC}iridium(III) (abbreviation: Ir(5mtpy-d₆)₂(mbfpypy-iPr-d₄)), [2-d₃-methyl-(2-pyridinyl-κN)benzofuro[2,3-b]pyridine-κC]bis[2-(2-pyridinyl-κN)phenyl-κC]iridium(III) (abbreviation: Ir(ppy)₂(mbfpypy-d₃)), [2-(4-methyl-5-phenyl-2-pyridinyl-κN)phenyl-κC]bis[2-(2-pyridinyl-κN)phenyl-κC]iridium(III) (abbreviation: Ir(ppy)₂(mdppy)), [2-(4-d₃-methyl-5-phenyl-2-pyridinyl-κN²)phenyl-κC]bis[2-(5-d₃-methyl-2-pyridinyl-N²)phenyl-κC]iridium(III) (abbreviation: [Ir(5mppy-d₃)₂(mdppy-d₃)]), and [2-methyl-(2-pyridinyl-κN)benzofuro[2,3-b]pyridine-κC]bis[2-(2-pyridinyl-κN)phenyl-κC]iridium(III) (abbreviation: [Ir(ppy)₂(mbfpypy)]); organometallic complexes such as bis(2,4-diphenyl-1,3-oxazolato-N,C^{2′})iridium(III) acetylacetonate (abbreviation: [Ir(dpo)₂(acac)]), bis{2-[4′-(perfluorophenyl)phenyl]pyridinato-N,C^{2′}}iridium(III) acetylacetonate (abbreviation: [Ir(p-PF-ph)₂(acac)]), and bis(2-phenylbenzothiazolato-N,C^{2′})iridium(III) acetylacetonate (abbreviation: [Ir(bt)₂(acac)]); and a rare earth metal complex such as tris(acetylacetonato) (monophenanthroline)terbium(III) (abbreviation: [Tb(acac)₃(Phen)]).

<<Phosphorescent Substance (from 570 nm to 750 nm, Yellow or Red)>>

As examples of a phosphorescent substance which emits yellow or red light and whose emission spectrum has a peak wavelength of greater than or equal to 570 nm and less than or equal to 750 nm, the following substances can be given.

Examples include organometallic complexes having a pyrimidine ring, such as (diisobutyrylmethanato)bis[4,6-bis(3-methylphenyl)pyrimidinato]iridium(III) (abbreviation: [Ir(5mdppm)₂(dibm)]), bis[4,6-bis(3-methylphenyl)pyrimidinato](dipivaloylmethanato)iridium(III) (abbreviation: [Ir(5mdppm)₂(dpm)]), and (dipivaloylmethanato)bis[4,6-di(naphthalen-1-yl)pyrimidinato]iridium(III) (abbreviation: [Ir(d1npm)₂(dpm)]); organometallic complexes having a pyrazine ring, such as (acetylacetonato)bis(2,3,5-triphenylpyrazinato)iridium(III) (abbreviation: [Ir(tppr)₂(acac)]), bis(2,3,5-triphenylpyrazinato)(dipivaloylmethanato)iridium(III) (abbreviation: [Ir(tppr)₂(dpm)]), bis{4,6-dimethyl-2-[3-(3,5-dimethylphenyl)-5-phenyl-2-pyrazinyl-κN]phenyl-κC}(2,6-dimethyl-3,5-heptanedionato-κ²O,O′)iridium(III) (abbreviation: [Ir(dmdppr-P)₂(dibm)]), bis{4,6-dimethyl-2-[5-(4-cyano-2,6-dimethylphenyl)-3-(3,5-dimethylphenyl)-2-pyrazinyl-κN]phenyl-κC}(2,2,6,6-tetramethyl-3,5-heptanedionato-κ²O,O′)iridium(III) (abbreviation: [Ir(dmdppr-dmCP)₂(dpm)]), bis{2-[5-(2,6-dimethylphenyl)-3-(3,5-dimethylphenyl)-2-pyrazinyl-κN]-4,6-dimethylphenyl-κC} (2,2′,6,6′-tetramethyl-3,5-heptanedionato-κ²O,O′)iridium(III) (abbreviation: [Ir(dmdppr-dmp)₂(dpm)]), (acetylacetonato)bis(2-methyl-3-phenylquinoxalinato-N,C^{2′}]iridium(III) (abbreviation: Ir(mpq)₂(acac)), (acetylacetonato)bis(2,3-diphenylquinoxalinato-N,C^{2′})iridium(III) (abbreviation: [Ir(dpq)₂(acac)]), and (acetylacetonato)bis[2,3-bis(4-fluorophenyl)quinoxalinato]iridium(III) (abbreviation: [Ir(Fdpq)₂(acac)]); organometallic complexes having a pyridine ring, such as tris(1-phenylisoquinolinato-N,C^{2′})iridium(III) (abbreviation: [Ir(piq)₃]), bis(1-phenylisoquinolinato-N,C^{2′})iridium(III) acetylacetonate (abbreviation: [Ir(piq)₂(acac)]), and bis[4,6-dimethyl-2-(2-quinolinyl-κN)phenyl-KC] (2,4-pentanedionato-κ²O,O′)iridium(III) (abbreviation: [Ir(dmpqn)₂(acac)]); a platinum complex such as 2,3,7,8,12,13,17,18-octaethyl-21H,23H-porphyrin platinum(II) (abbreviation: [PtOEP]); and rare earth metal complexes such as tris(1,3-diphenyl-1,3-propanedionato)(monophenanthroline)europium(III) (abbreviation: [Eu(DBM)₃(Phen)]) and tris[1-(2-thenoyl)-3,3,3-trifluoroacetonato] (monophenanthroline)europium(III) (abbreviation: [Eu(TTA)₃(Phen)]).

For the other components in this structure example, refer to the structure of the light-emitting device described in Embodiment 2.

The structure described in this embodiment can be used in combination with any of the structures described in the other embodiments as appropriate.

### Embodiment 4

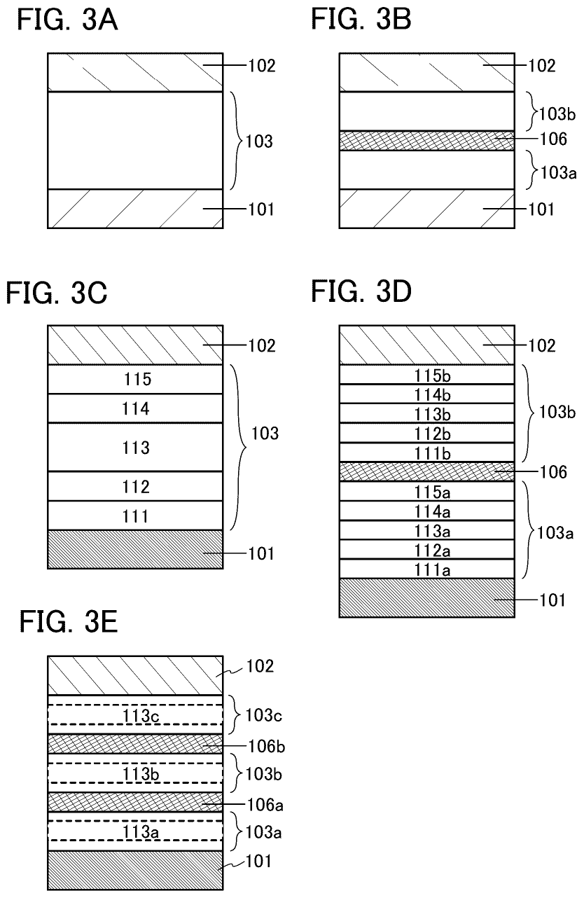

In this embodiment, structures of layers other than the light-emitting layer of the light-emitting device described in Embodiments 2 and 3 will be described with reference to FIGS. 3A to 3E.

### <<Basic Structure of Light-Emitting Device>>

Basic structures of the light-emitting device will be described. As described in Embodiment 2, FIG. 3A illustrates a light-emitting device including, between the pair of electrodes, the EL layer 103 including the light-emitting layer.

FIG. 3B illustrates a light-emitting device having a structure in which a plurality of EL layers (two EL layers of 103a and 103b in FIG. 3B) are provided between the pair of electrodes and a charge-generation layer 106 is provided between the EL layers (such a structure is also referred to as a tandem structure). A light-emitting device having a tandem structure enables fabrication of a light-emitting apparatus that has high efficiency without changing the amount of current.

The charge-generation layer 106 has a function of injecting electrons into one of the EL layers 103a and 103b and injecting holes into the other of the EL layers 103a and 103b when a potential difference is caused between the first electrode 101 and the second electrode 102. Thus, when voltage is applied in FIG. 3B such that the potential of the first electrode 101 can be higher than that of the second electrode 102, electrons are injected into the EL layer 103a from the charge-generation layer 106 and holes are injected into the EL layer 103b from the charge-generation layer 106.

Note that in terms of light extraction efficiency, the charge-generation layer 106 preferably has a property of transmitting visible light (specifically, the charge-generation layer 106 preferably has a visible light transmittance higher than or equal to 40%). The charge-generation layer 106 functions even if it has lower conductivity than the first electrode 101 and the second electrode 102.

FIG. 3C illustrates a stacked-layer structure of the EL layer 103 in the light-emitting device. In this case, the first electrode 101 is regarded as functioning as an anode and the second electrode 102 is regarded as functioning as a cathode. The EL layer 103 has a structure in which a hole-injection layer 111, a hole-transport layer 112, the light-emitting layer 113, an electron-transport layer 114, and an electron-injection layer 115 are sequentially stacked over the first electrode 101. Note that the first electrode 101 may function as a cathode, and the second electrode 102 may function as an anode. In that case, the stacking order of the layers in the EL layer 103 is preferably reversed; specifically, it is preferable that the layer 111 over the first electrode 101 functioning as the cathode be an electron-injection layer, the layer 112 be an electron-transport layer, the layer 113 be a light-emitting layer, the layer 114 be a hole-transport layer, and the layer 115 be a hole-injection layer.

The light-emitting layer 113 contains an appropriate combination of a light-emitting substance and a plurality of substances, so that fluorescent light of a desired color or phosphorescent light of a desired color can be obtained. The light-emitting layer of the light-emitting device of one embodiment of the present invention preferably employs any of the structures of the light-emitting layer described in Embodiments 2 and 3.

Note that the light-emitting layer 113 may have a stacked-layer structure of a plurality of light-emitting layers that emit light of different colors. When a plurality of light-emitting layers are provided, the use of different light-emitting substances for the light-emitting layers enables a structure that exhibits different emission colors (for example, complementary emission colors are combined to obtain white light emission). For example, a light-emitting layer containing a light-emitting substance that emits red light, a light-emitting layer containing a light-emitting substance that emits green light, and a light-emitting layer containing a light-emitting substance that emits blue light may be stacked with or without a layer containing a carrier-transport material therebetween. Alternatively, a light-emitting layer containing a light-emitting substance that emits yellow light and a light-emitting layer containing a light-emitting substance that emits blue light may be used in combination. In this case, the combination of the light-emitting substance and other substances are different between the stacked light-emitting layers. Alternatively, the plurality of EL layers (103a and 103b) in FIG. 3B may exhibit their respective emission colors. Also in this case, the light-emitting substances and other substances are different between the stacked light-emitting layers.

Note that the stacked-layer structure of the light-emitting layer 113 is not limited to the above. For example, the light-emitting layer 113 may have a stacked-layer structure of a plurality of light-emitting layers that emit light of the same color. For example, a first light-emitting layer containing a light-emitting substance that emits blue light and a second light-emitting layer containing a light-emitting substance that emits blue light may be stacked with or without a layer containing a carrier-transport material therebetween. Alternatively, the plurality of EL layers (103a and 103b) in FIG. 3B may exhibit the same emission color. The structure in which a plurality of light-emitting layers that emit light of the same color are stacked can sometimes achieve higher reliability than a single-layer structure.

In the case where the light-emitting layer 113 has a structure in which a plurality of light-emitting layers are stacked, at least one of the plurality of light-emitting layers preferably employs any of the structures of the light-emitting layer described in Embodiments 2 and 3.

The light-emitting device can have a micro optical resonator (microcavity) structure when, for example, the first electrode 101 is a reflective electrode and the second electrode 102 is a transflective electrode in FIG. 3C. Thus, light from the light-emitting layer 113 in the EL layer 103 can be resonated between the electrodes and light emitted through the second electrode 102 can be intensified.

Note that when the first electrode 101 of the light-emitting device is a reflective electrode having a stacked-layer structure of a reflective conductive material and a light-transmitting conductive material (transparent conductive film), optical adjustment can be performed by adjusting the thickness of the transparent conductive film. Specifically, when the wavelength of light obtained from the light-emitting layer 113 is λ, the optical path length between the first electrode 101 and the second electrode 102 (the product of the thickness and the refractive index) is preferably adjusted to be mλ/2 (m is an integer of 1 or more) or close to mλ/2.

To amplify desired light (wavelength: k) obtained from the light-emitting layer 113, it is preferable to adjust each of the optical path length from the first electrode 101 to a region where the desired light is obtained in the light-emitting layer 113 (light-emitting region) and the optical path length from the second electrode 102 to the region where the desired light is obtained in the light-emitting layer 113 (light-emitting region) to be (2m′+1)λ/4 (m′ is an integer of 1 or more) or close to (2m′+1)λ/4. Here, the light-emitting region means a region where holes and electrons are recombined in the light-emitting layer 113.

By such optical adjustment, the spectrum of specific monochromatic light obtained from the light-emitting layer 113 can be narrowed and light emission with high color purity can be obtained.

In the above case, the optical path length between the first electrode 101 and the second electrode 102 is, to be exact, the total thickness from a reflective region in the first electrode 101 to a reflective region in the second electrode 102. However, it is difficult to precisely determine the reflective regions in the first electrode 101 and the second electrode 102; thus, it is assumed that the above effect can be sufficiently obtained wherever the reflective regions may be set in the first electrode 101 and the second electrode 102. Furthermore, the optical path length between the first electrode 101 and the light-emitting layer that emits the desired light is, to be exact, the optical path length between the reflective region in the first electrode 101 and the light-emitting region in the light-emitting layer that emits the desired light. However, it is difficult to precisely determine the reflective region in the first electrode 101 and the light-emitting region in the light-emitting layer that emits the desired light; thus, it is assumed that the above effect can be sufficiently obtained wherever the reflective region and the light-emitting region may be set in the first electrode 101 and the light-emitting layer that emits the desired light, respectively.

FIG. 3D illustrates a stacked-layer structures of the EL layers (103a and 103b) of the light-emitting device having a tandem structure. In this case, the first electrode 101 is regarded as functioning as an anode and the second electrode 102 is regarded as functioning as a cathode. The EL layer 103a has a structure in which a hole-injection layer 11a, a hole-transport layer 112a, a light-emitting layer 113a, an electron-transport layer 114a, and an electron-injection layer 115a are sequentially stacked over the first electrode 101. The EL layer 103b has a structure in which a hole-injection layer 1l1b, a hole-transport layer 112b, a light-emitting layer 113b, an electron-transport layer 114b, and an electron-injection layer 115b are sequentially stacked over the charge-generation layer 106. Note that the first electrode 101 may function as a cathode and the second electrode 102 may function as an anode; in this case, the stacking order of the layers in the EL layer 103 is preferably reversed.

For example, when the light-emitting device in FIG. 3D has a microcavity structure, the first electrode 101 is formed as a reflective electrode and the second electrode 102 is formed as a transflective electrode. Thus, a single-layer structure or a stacked-layer structure can be formed using one or more kinds of desired electrode materials. Note that the second electrode 102 is formed after formation of the EL layer 103b, with the use of a material selected as appropriate.

In the case where the light-emitting device illustrated in FIG. 3D has a microcavity structure and light-emitting layers that emit light of different colors are used in the EL layers (103a and 103b), light (monochromatic light) with a desired wavelength derived from any of the light-emitting layers can be extracted owing to the microcavity structure. When such a light-emitting device is used for the light-emitting apparatus and the microcavity structure is adjusted in order to extract light with wavelengths which differ among pixels, separate formation of EL layers for obtaining different emission colors (e.g., R, G, and B) for each pixel is unnecessary. Therefore, higher resolution can be easily achieved. A combination with coloring layers (color filters) is also possible. Furthermore, the emission intensity of light with a specific wavelength in the front direction can be increased, whereby power consumption can be reduced.

The light-emitting device illustrated in FIG. 3E is an example of the light-emitting device having the tandem structure illustrated in FIG. 3B, and includes three EL layers (103a, 103b, and 103c) stacked with charge-generation layers (106a and 106b) therebetween, as illustrated in FIG. 3E. The three EL layers (103a, 103b, and 103c) include respective light-emitting layers (113a, 113b, and 113c), and the emission colors of the light-emitting layers can be selected freely. For example, the light-emitting layer 113a can emit blue light, the light-emitting layer 113b can emit red light, green light, or yellow light, and the light-emitting layer 113c can emit blue light. For another example, the light-emitting layer 113a can emit red light, the light-emitting layer 113b can emit blue light, green light, or yellow light, and the light-emitting layer 113c can emit red light.

In the light-emitting device of one embodiment of the present invention, at least one of the first electrode 101 and the second electrode 102 is a light-transmitting electrode (e.g., a transparent electrode or a transflective electrode). In the case where the light-transmitting electrode is a transparent electrode, the transparent electrode has a visible light transmittance higher than or equal to 40%. In the case where the light-transmitting electrode is a transflective electrode, the transflective electrode has a visible light reflectance higher than or equal to 20% and lower than or equal to 80%, preferably higher than or equal to 40% and lower than or equal to 70%. These electrodes preferably have a resistivity less than or equal to 1×10⁻² Ωcm.

When one of the first electrode 101 and the second electrode 102 is a reflective electrode in the light-emitting device of one embodiment of the present invention, the visible light reflectance of the reflective electrode is higher than or equal to 40% and lower than or equal to 100%, preferably higher than or equal to 70% and lower than or equal to 100%. This electrode preferably has a resistivity less than or equal to 1×10⁻² Ωm.

### <<Specific Structure of Light-Emitting Device>>

Next, specific structures of layers in the light-emitting device of one embodiment of the present invention will be described. Note that for simplicity, reference numerals are sometimes omitted in the description of the layers.

### <First Electrode and Second Electrode>

As materials for the first electrode and the second electrode, any of the following materials can be used in an appropriate combination as long as the above functions of the electrodes can be fulfilled. For example, a metal, an alloy, an electrically conductive compound, a mixture of these, and the like can be used as appropriate. Specifically, an In—Sn oxide (also referred to as ITO), an In—Si—Sn oxide (also referred to as ITSO), an In—Zn oxide, or an In—W—Zn oxide can be used. In addition, it is possible to use a metal such as aluminum (Al), titanium (Ti), chromium (Cr), manganese (Mn), iron (Fe), cobalt (Co), nickel (Ni), copper (Cu), gallium (Ga), zinc (Zn), indium (In), tin (Sn), molybdenum (Mo), tantalum (Ta), tungsten (W), palladium (Pd), gold (Au), platinum (Pt), silver (Ag), yttrium (Y), or neodymium (Nd) or an alloy containing an appropriate combination of any of these metals. It is also possible to use an element belonging to Group 1 or Group 2 of the periodic table that is not described above (e.g., lithium (Li), cesium (Cs), calcium (Ca), or strontium (Sr)), a rare earth metal such as europium (Eu) or ytterbium (Yb), an alloy containing an appropriate combination of any of these elements, graphene, or the like.

In the light-emitting device illustrated in FIG. 3D, when the first electrode 101 is the anode, the hole-injection layer 111a and the hole-transport layer 112a of the EL layer 103a are sequentially stacked over the first electrode 101 by a vacuum evaporation method. After the EL layer 103a and the charge-generation layer 106 are formed, the hole-injection layer 111b and the hole-transport layer 112b of the EL layer 103b are sequentially stacked over the charge-generation layer 106 in a similar manner.

### <Hole-Injection Layer>

The hole-injection layer injects holes from the first electrode that is an anode and the charge-generation layer to the EL layer, and contains an organic acceptor material and a material having a high hole-injection property.

The organic acceptor material allows holes to be generated in another organic compound whose HOMO level is close to the LUMO level of the organic acceptor material when charge separation is caused between the organic acceptor material and the organic compound. Thus, as the organic acceptor material, a compound having an electron-withdrawing group (e.g., a halogen group or a cyano group), such as a quinodimethane derivative, a chloranil derivative, and a hexaazatriphenylene derivative, can be used. For example, any of the following materials can be used: 7,7,8,8-tetracyano-2,3,5,6-tetrafluoroquinodimethane (abbreviation: F₄-TCNQ), 3,6-difluoro-2,5,7,7,8,8-hexacyanoquinodimethane, chloranil, 2,3,6,7,10,11-hexacyano-1,4,5,8,9,12-hexaazatriphenylene (abbreviation: HAT-CN), 1,3,4,5,7,8-hexafluorotetracyano-naphthoquinodimethane (abbreviation: F₆-TCNNQ), and 2-(7-dicyanomethylene-1,3,4,5,6,8,9,10-octafluoro-7H-pyren-2-ylidene)malononitrile. Note that among organic acceptor materials, a compound in which electron-withdrawing groups are bonded to condensed aromatic rings each having a plurality of heteroatoms, such as HAT-CN, is particularly preferred because it has a high acceptor property and stable film quality against heat. Besides, a [3]radialene derivative having an electron-withdrawing group (particularly a cyano group or a halogen group such as a fluoro group), which has a very high electron-accepting property, is preferred; specific examples include α,α′,α″-1,2,3-cyclopropanetriylidenetris[4-cyano-2,3,5,6-tetrafluorobenzeneacetonitrile], α,α′,α″-1,2,3-cyclopropanetriylidenetris[2,6-dichloro-3,5-difluoro-4-(trifluoromethyl)benzeneacetonitrile], and α,α′,α″-1,2,3-cyclopropanetriylidenetris[2,3,4,5,6-pentafluorobenzeneacetonitrile].

As the material having a high hole-injection property, an oxide of a metal belonging to Group 4 to Group 8 of the periodic table (e.g., a transition metal oxide such as molybdenum oxide, vanadium oxide, ruthenium oxide, tungsten oxide, or manganese oxide) can be used. Specific examples include molybdenum oxide, vanadium oxide, niobium oxide, tantalum oxide, chromium oxide, tungsten oxide, manganese oxide, and rhenium oxide. Among the above oxides, molybdenum oxide is preferable because it is stable in the air, has a low hygroscopic property, and is easily handled. Other examples include phthalocyanine (abbreviation: H₂Pc) and a phthalocyanine-based compound such as copper phthalocyanine (abbreviation: CuPc).

Other examples include aromatic amine compounds, which are low molecular compounds, such as 4,4′,4″-tris(N,N-diphenylamino)triphenylamine (abbreviation: TDATA), 4,4′,4″-tris[N-(3-methylphenyl)-N-phenylamino]triphenylamine (abbreviation: MTDATA), 4,4′-bis[N-(4-diphenylaminophenyl)-N-phenylamino]biphenyl (abbreviation: DPAB), N,N-bis[4-bis(3-methylphenyl)aminophenyl]-N,N-diphenyl-4,4′-diaminobiphenyl (abbreviation: DNTPD), 1,3,5-tris[N-(4-diphenylaminophenyl)-N-phenylamino]benzene (abbreviation: DPA3B), 3-[N-(9-phenylcarbazol-3-yl)-N-phenylamino]-9-phenylcarbazole (abbreviation: PCzPCA1), 3,6-bis[N-(9-phenylcarbazol-3-yl)-N-phenylamino]-9-phenylcarbazole (abbreviation: PCzPCA2), and 3-[N-(1-naphthyl)-N-(9-phenylcarbazol-3-yl)amino]-9-phenylcarbazole (abbreviation: PCzPCN1).

Other examples include high molecular compounds (e.g., oligomers, dendrimers, and polymers) such as poly(N-vinylcarbazole) (abbreviation: PVK), poly(4-vinyltriphenylamine) (abbreviation: PVTPA), poly[N-(4-{N¹-[4-(4-diphenylamino)phenyl]phenyl-N-phenylamino}phenyl)methacrylamide] (abbreviation: PTPDMA), and poly[N,N-bis(4-butylphenyl)-N,N-bis(phenyl)benzidine] (abbreviation: Poly-TPD). Other examples include a high molecular compound to which acid is added, such as poly(3,4-ethylenedioxythiophene)/poly(styrenesulfonic acid) (abbreviation: PEDOT/PSS) and polyaniline/poly(styrenesulfonic acid) (abbreviation: PAni/PSS).

As the material having a high hole-injection property, a mixed material containing a hole-transport material and the above-described organic acceptor material (electron-accepting material) can be used. In this case, the organic acceptor material extracts electrons from the hole-transport material, so that holes are generated in the hole-injection layer 111 and injected into the light-emitting layer 113 through the hole-transport layer 112. Note that the hole-injection layer 111 may be formed to have a single-layer structure using a mixed material containing a hole-transport material and an organic acceptor material (electron-accepting material), or a stacked-layer structure of a layer containing a hole-transport material and a layer containing an organic acceptor material (electron-accepting material).

The hole-transport material preferably has a hole mobility higher than or equal to 1×10⁻⁶ cm²/Vs in the case where the square root of the electric field strength [V/cm] is 600. Note that any other substance can also be used as long as the substance has a hole-transport property higher than an electron-transport property.

As the hole-transport material, a material having a high hole-transport property, such as a compound having a π-electron rich heteroaromatic ring (e.g., a carbazole derivative, a furan derivative, or a thiophene derivative) or an aromatic amine (an organic compound having an aromatic amine skeleton), is preferable.

Examples of the carbazole derivative (an organic compound having a carbazole ring) include a bicarbazole derivative (e.g., a 3,3′-bicarbazole derivative) and an aromatic amine having a carbazolyl group.

Specific examples of the bicarbazole derivative (e.g., a 3,3′-bicarbazole derivative) include 3,3′-bis(9-phenyl-9H-carbazole) (abbreviation: PCCP), 9,9′-bis(biphenyl-4-yl)-3,3′-bi-9H-carbazole (abbreviation: BisBPCz), 9,9′-bis(biphenyl-3-yl)-3,3′-bi-9H-carbazole (abbreviation: BismBPCz), 9-(biphenyl-3-yl)-9′-(biphenyl-4-yl)-9H,9′H-3,3′-bicarbazole (abbreviation: mBPCCBP), and 9-(2-naphthyl)-9′-phenyl-9H,9′H-3,3′-bicarbazole (abbreviation: ONCCP).

Specific examples of the aromatic amine having a carbazolyl group include 4-phenyl-4′-(9-phenyl-9H-carbazol-3-yl)triphenylamine (abbreviation: PCBA1BP), N-(4-biphenyl)-N-(9,9-dimethyl-9H-fluoren-2-yl)-9-phenyl-9H-carbazol-3-amine (abbreviation: PCBiF), N-(biphenyl-4-yl)-N-[4-(9-phenyl-9H-carbazol-3-yl)phenyl]-9,9-dimethyl-9H-fluoren-2-amine (abbreviation: PCBBiF), N-[4-(9-phenyl-9H-carbazol-3-yl)phenyl]bis(9,9-dimethyl-9H-fluoren-2-yl)amine (abbreviation: PCBFF), N-(1,1′-biphenyl-4-yl)-N-[4-(9-phenyl-9H-carbazol-3-yl)phenyl]-9,9-dimethyl-9H-fluoren-4-amine, N-[4-(9-phenyl-9H-carbazol-3-yl)phenyl]-(9,9-dimethyl-9H-fluoren-2-yl)-9,9-dimethyl-9H-fluoren-4-amine, N-(1,1′-biphenyl-4-yl)-N-[4-(9-phenyl-9H-carbazol-3-yl)phenyl]-9,9-diphenyl-9H-fluoren-2-amine, N-(1,1′-biphenyl-4-yl)-N-[4-(9-phenyl-9H-carbazol-3-yl)phenyl]-9,9-diphenyl-9H-fluoren-4-amine, N-(1,1′-biphenyl-4-yl)-N-[4-(9-phenyl-9H-carbazol-3-yl)phenyl]-9,9′-spirobi(9H-fluoren)-2-amine, N-(1,1′-biphenyl-4-yl)-N-[4-(9-phenyl-9H-carbazol-3-yl)phenyl]-9,9′-spirobi(9H-fluoren)-4-amine, N-[4-(9-phenyl-9H-carbazol-3-yl)phenyl]-N-(1,1′: 3′,1″-terphenyl-4-yl)-9,9-dimethyl-9H-fluoren-2-amine, N-[4-(9-phenyl-9H-carbazol-3-yl)phenyl]-N-(1,1′: 4′,1″-terphenyl-4-yl)-9,9-dimethyl-9H-fluoren-2-amine, N-[4-(9-phenyl-9H-carbazol-3-yl)phenyl]-N-(1,1′: 3′,1″-terphenyl-4-yl)-9,9-dimethyl-9H-fluoren-4-amine, N-[4-(9-phenyl-9H-carbazol-3-yl)phenyl]-N-(1,1′: 4′,1″-terphenyl-4-yl)-9,9-dimethyl-9H-fluoren-4-amine, 4,4′-diphenyl-4″-(9-phenyl-9H-carbazol-3-yl)triphenylamine (abbreviation: PCBBi1BP), 4-(1-naphthyl)-4′-(9-phenyl-9H-carbazol-3-yl)triphenylamine (abbreviation: PCBANB), 4,4′-di(1-naphthyl)-4″-(9-phenyl-9H-carbazol-3-yl)triphenylamine (abbreviation: PCBNBB), 4-phenyldiphenyl-(9-phenyl-9H-carbazol-3-yl)amine (abbreviation: PCA1BP), N,N-bis(9-phenylcarbazol-3-yl)-N,N-diphenylbenzene-1,3-diamine (abbreviation: PCA2B), N,N′,N″-triphenyl-N,N′,N″-tris(9-phenylcarbazol-3-yl)benzene-1,3,5-triamine (abbreviation: PCA3B), 9,9-dimethyl-N-phenyl-N-[4-(9-phenyl-9H-carbazol-3-yl)phenyl]fluoren-2-amine (abbreviation: PCBAF), N-phenyl-N-[4-(9-phenyl-9H-carbazol-3-yl)phenyl]-9,9′-spirobi[9H-fluoren]-2-amine (abbreviation: PCBASF), PCzPCA1, PCzPCA2, PCzPCN1, 3-[N-(4-diphenylaminophenyl)-N-phenylamino]-9-phenylcarbazole (abbreviation: PCzDPA1), 3,6-bis[N-(4-diphenylaminophenyl)-N-phenylamino]-9-phenylcarbazole (abbreviation: PCzDPA2), 3,6-bis[N-(4-diphenylaminophenyl)-N-(1-naphthyl)amino]-9-phenylcarbazole (abbreviation: PCzTPN2), 2-[N-(9-phenylcarbazol-3-yl)-N-phenylamino]spiro-9,9′-bifluorene (abbreviation: PCASF), N-[4-(9H-carbazol-9-yl)phenyl]-N-(4-phenyl)phenylaniline (abbreviation: YGA1BP), N,N-bis[4-(carbazol-9-yl)phenyl]-N,N-diphenyl-9,9-dimethylfluorene-2,7-diamine (abbreviation: YGA2F), and 4,4′,4″-tris(carbazol-9-yl)triphenylamine (abbreviation: TCTA).

Other examples of the carbazole derivative include 9-[4-(9-phenyl-9H-carbazol-3-yl)-phenyl]phenanthrene (abbreviation: PCPPn), 3-[4-(1-naphthyl)-phenyl]-9-phenyl-9H-carbazole (abbreviation: PCPN), 1,3-bis(N-carbazolyl)benzene (abbreviation: mCP), 4,4′-di(N-carbazolyl)biphenyl (abbreviation: CBP), 3,6-bis(3,5-diphenylphenyl)-9-phenylcarbazole (abbreviation: CzTP), 1,3,5-tris[4-(N-carbazolyl)phenyl]benzene (abbreviation: TCPB), and 9-[4-(10-phenyl-9-anthracenyl)phenyl]-9H-carbazole (abbreviation: CzPA).

Specific examples of the furan derivative (an organic compound having a furan ring) include 4,4′,4″-(benzene-1,3,5-triyl)tri(dibenzofuran) (abbreviation: DBF3P-II) and 4-{3-[3-(9-phenyl-9H-fluoren-9-yl)phenyl]phenyl}dibenzofuran (abbreviation: mmDBFFLBi-II).

Specific examples of the thiophene derivative (an organic compound having a thiophene ring) include organic compounds having a thiophene ring, such as 4,4′,4″-(benzene-1,3,5-triyl)tri(dibenzothiophene) (abbreviation: DBT3P-II), 2,8-diphenyl-4-[4-(9-phenyl-9H-fluoren-9-yl)phenyl]dibenzothiophene (abbreviation: DBTFLP-III), and 4-[4-(9-phenyl-9H-fluoren-9-yl)phenyl]-6-phenyldibenzothiophene (abbreviation: DBTFLP-IV).

Specific examples of the aromatic amine include 4,4′-bis[N-(1-naphthyl)-N-phenylamino]biphenyl (abbreviation: NPB or α-NPD), N,N′-diphenyl-N,N-bis(3-methylphenyl)-4,4′-diaminobiphenyl (abbreviation: TPD), N,N-bis(9,9′-spirobi[9H-fluoren]-2-yl)-N,N-diphenyl-4,4′-diaminobiphenyl (abbreviation: BSPB), 4-phenyl-4′-(9-phenylfluoren-9-yl)triphenylamine (abbreviation: BPAFLP), 4-phenyl-3′-(9-phenylfluoren-9-yl)triphenylamine (abbreviation: mBPAFLP), N-(9,9-dimethyl-9H-fluoren-2-yl)-N-{9,9-dimethyl-2-[N¹-phenyl-N-(9,9-dimethyl-9H-fluoren-2-yl)amino]-9H-fluoren-7-yl}phenylamine (abbreviation: DFLADFL), N-(9,9-dimethyl-2-diphenylamino-9H-fluoren-7-yl)diphenylamine (abbreviation: DPNF), 2-[N-(4-diphenylaminophenyl)-N-phenylamino]-spiro-9,9′-bifluorene (abbreviation: DPASF), 2,7-bis[N-(4-diphenylaminophenyl)-N-phenylamino]-spiro-9,9′-bifluorene (abbreviation: DPA2SF), 4,4′,4″-tris[N-(1-naphthyl)-N-phenylamino]triphenylamine (abbreviation: 1′-TNATA), TDATA, 4,4′,4″-tris[N-(3-methylphenyl)-N-phenylamino]triphenylamine (abbreviation: m-MTDATA), N,N-di(p-tolyl)-N,N′-diphenyl-p-phenylenediamine (abbreviation: DTDPPA), DPAB, DNTPD, DPA3B, N-(4-biphenyl)-6,N-diphenylbenzo[b]naphtho[1,2-d]furan-8-amine (abbreviation: BnfABP), N,N-bis(4-biphenyl)-6-phenylbenzo[b]naphtho[1,2-d]furan-8-amine (abbreviation: BBABnf), 4,4′-bis(6-phenylbenzo[b]naphtho[1,2-d]furan-8-yl)-4″-phenyltriphenylamine (abbreviation: BnfBB1BP), N,N-bis(4-biphenyl)benzo[b]naphtho[1,2-d]furan-6-amine (abbreviation: BBABnf(6)), N,N-bis(4-biphenyl)benzo[b]naphtho[1,2-d]furan-8-amine (abbreviation: BBABnf(8)), N,N-bis(4-biphenyl)benzo[b]naphtho[2,3-d]furan-4-amine (abbreviation: BBABnf(II)(4)), N,N-bis[4-(dibenzofuran-4-yl)phenyl]-4-amino-p-terphenyl (abbreviation: DBfBB1TP), N-[4-(dibenzothiophen-4-yl)phenyl]-N-phenyl-4-biphenylamine (abbreviation: ThBA1BP), 4-(2-naphthyl)-4′,4″-diphenyltriphenylamine (abbreviation: BBAβNB), 4-[4-(2-naphthyl)phenyl]-4′,4″-diphenyltriphenylamine (abbreviation: BBAβNBi), 4,4′-diphenyl-4″-(6;1′-binaphthyl-2-yl)triphenylamine (abbreviation: BBAαNβNB), 4,4′-diphenyl-4″-(7;1′-binaphthyl-2-yl)triphenylamine (abbreviation: BBAαNβNB-03), 4,4′-diphenyl-4″-(7-phenyl)naphthyl-2-yltriphenylamine (abbreviation: BBAPβNB-03), 4,4′-diphenyl-4″-(6;2′-binaphthyl-2-yl)triphenylamine (abbreviation: BBA(βN2)B), 4,4′-diphenyl-4″-(7;2′-binaphthyl-2-yl)triphenylamine (abbreviation: BBA(βN2)B-03), 4,4′-diphenyl-4″-(4;2′-binaphthyl-1-yl)triphenylamine (abbreviation: BBAβNαNB), 4,4′-diphenyl-4″-(5;2′-binaphthyl-1-yl)triphenylamine (abbreviation: BBAβNαNB-02), 4-(4-biphenylyl)-4′-(2-naphthyl)-4″-phenyltriphenylamine (abbreviation: TPBiAβNB), 4-(3-biphenylyl)-4′-[4-(2-naphthyl)phenyl]-4″-phenyltriphenylamine (abbreviation: mTPBiAβNBi), 4-(4-biphenylyl)-4′-[4-(2-naphthyl)phenyl]-4″-phenyltriphenylamine (abbreviation: TPBiAβNBi), 4-phenyl-4′-(1-naphthyl)triphenylamine (abbreviation: αNBA1BP), 4,4′-bis(1-naphthyl)triphenylamine (abbreviation: αNBB1BP), 4,4′-diphenyl-4″-[4′-(carbazol-9-yl)biphenyl-4-yl]triphenylamine (abbreviation: YGTBi1BP), 4′-[4-(3-phenyl-9H-carbazol-9-yl)phenyl]tris(biphenyl-4-yl)amine (abbreviation: YGTBi1BP-02), 4-[4′-(carbazol-9-yl)biphenyl-4-yl]-4′-(2-naphthyl)-4″-phenyltriphenylamine (abbreviation: YGTBiβNB), N-[4-(9-phenyl-9H-carbazol-3-yl)phenyl]-N-[4-(1-naphthyl)phenyl]-9,9′-spirobi[9H-fluoren]-2-amine (abbreviation: PCBNBSF), N,N-bis(biphenyl-4-yl)-9,9′-spirobi[9H-fluoren]-2-amine (abbreviation: BBASF), N,N-bis(biphenyl-4-yl)-9,9′-spirobi[9H-fluoren]-4-amine (abbreviation: BBASF(4)), N-(biphenyl-2-yl)-N-(9,9-dimethyl-9H-fluoren-2-yl)-9,9′-spirobi[9H-fluoren]-4-amine (abbreviation: oFBiSF), N-(biphenyl-4-yl)-N-(9,9-dimethyl-9H-fluoren-2-yl)dibenzofuran-4-amine (abbreviation: FrBiF), N-[4-(1-naphthyl)phenyl]-N-[3-(6-phenyldibenzofuran-4-yl)phenyl]-1-naphthylamine (abbreviation: mPDBfBNBN), 4-phenyl-4′-[4-(9-phenylfluoren-9-yl)phenyl]triphenylamine (abbreviation: BPAFLBi), N,N-bis(9,9-dimethyl-9H-fluoren-2-yl)-9,9′-spirobi-9H-fluoren-4-amine, N,N-bis(9,9-dimethyl-9H-fluoren-2-yl)-9,9′-spirobi-9H-fluoren-3-amine, N,N-bis(9,9-dimethyl-9H-fluoren-2-yl)-9,9′-spirobi-9H-fluoren-2-amine, and N,N-bis(9,9-dimethyl-9H-fluoren-2-yl)-9,9′-spirobi-9H-fluoren-1-amine.

Other than the above, PVK, PVTPA, PTPDMA, Poly-TPD, or the like that is a high molecular compound (e.g., an oligomer, a dendrimer, or a polymer) can be used as the hole-transport material. Alternatively, a high molecular compound to which acid is added, such as PEDOT/PSS or PAni/PSS can be used, for example.

Note that the hole-transport material is not limited to the above examples, and any of a variety of known materials may be used alone or in combination as the hole-transport material.

The hole-injection layer can be formed by any of known deposition methods such as a vacuum evaporation method.

### <Hole-Transport Layer>

The hole-transport layer transports holes, which are injected from the first electrode 101 by the hole-injection layer, to the light-emitting layer. The hole-transport layer contains a hole-transport material. Thus, the hole-transport layer can be formed using a hole-transport material that can be used for the hole-injection layer. Furthermore, the hole-transport layer can function even with a single-layer structure, but may have a stacked structure of two or more layers. For example, one of two hole-transport layers that is in contact with the light-emitting layer may also function as an electron-blocking layer.

Note that in the light-emitting device of one embodiment of the present invention, the same organic compound can be used for the hole-transport layer and the light-emitting layer. Using the same organic compound for the hole-transport layer and the light-emitting layer is preferable because holes can be efficiently transported from the hole-transport layer to the light-emitting layer.

### <Electron-Transport Layer>

The electron-transport layer transports electrons, which are injected from the second electrode and the charge-generation layer by the electron-injection layer to be described later, to the light-emitting layer. The material used for the electron-transport layer is preferably a substance having an electron mobility higher than or equal to 1×10⁻⁶ cm²/Vs in the case where the square root of the electric field strength [V/cm] is 600. Note that any other substance can also be used as long as the substance has an electron-transport property higher than a hole-transport property. Furthermore, the electron-transport layer can function even with a single-layer structure, but may have a stacked structure of two or more layers. For example, one of two electron-transport layers that is in contact with the light-emitting layer may also function as a hole-blocking layer. Moreover, when the electron-transport layer has a stacked-layer structure, heat resistance can be increased in some cases. A photolithography process performed over the electron-transport layer including the above-described mixed material, which has heat resistance, can inhibit an adverse effect of thermal process on the device characteristics.

### <<Electron-Transport Material>>

As the electron-transport material that can be used for the electron-transport layer, an organic compound having a high electron-transport property can be used, and for example, a heteroaromatic compound can be used. The heteroaromatic compound refers to a cyclic compound containing at least two different kinds of elements in a ring. Examples of cyclic structures include a three-membered ring, a four-membered ring, a five-membered ring, and a six-membered ring, among which a five-membered ring and a six-membered ring are particularly preferable. The elements contained in the heteroaromatic compound are preferably one or more of nitrogen, oxygen, and sulfur, in addition to carbon. In particular, a heteroaromatic compound containing nitrogen (a nitrogen-containing heteroaromatic compound) is preferable, and any of materials having a high electron-transport property (electron-transport materials), such as a nitrogen-containing heteroaromatic compound and a π-electron deficient heteroaromatic compound including the nitrogen-containing heteroaromatic compound, is preferably used.

Note that the electron-transport material can be different from the materials used for the light-emitting layer. Not all excitons formed by recombination of carriers in the light-emitting layer can contribute to light emission and some excitons might be diffused into a layer in contact with the light-emitting layer or a layer in the vicinity of the light-emitting layer. In order to avoid this phenomenon, the energy level (the lowest singlet excitation energy level or the lowest triplet excitation energy level) of a material used for the layer in contact with the light-emitting layer or the layer in the vicinity of the light-emitting layer is preferably higher than that of a material used for the light-emitting layer. Therefore, when a material different from the material of the light-emitting layer is used as the electron-transport material, an element with high efficiency can be obtained.

The heteroaromatic compound is an organic compound having at least one heteroaromatic ring.

The heteroaromatic ring has any one of a pyridine ring, a diazine ring, a triazine ring, a polyazole ring, an oxazole ring, a thiazole ring, and the like. A heteroaromatic ring having a diazine ring includes a heteroaromatic ring having a pyrimidine ring, a pyrazine ring, a pyridazine ring, or the like. A heteroaromatic ring having a polyazole ring includes a heteroaromatic ring having an imidazole ring, a triazole ring, or an oxadiazole ring.

The heteroaromatic ring includes a condensed heteroaromatic ring having a fused ring structure. Examples of the condensed heteroaromatic ring include a quinoline ring, a benzoquinoline ring, a quinoxaline ring, a dibenzoquinoxaline ring, a quinazoline ring, a benzoquinazoline ring, a dibenzoquinazoline ring, a phenanthroline ring, a furodiazine ring, and a benzimidazole ring.

Examples of the heteroaromatic compound having a five-membered ring structure, which is a heteroaromatic compound containing carbon and one or more of nitrogen, oxygen, sulfur, and the like, include a heteroaromatic compound having an imidazole ring, a heteroaromatic compound having a triazole ring, a heteroaromatic compound having an oxazole ring, a heteroaromatic compound having an oxadiazole ring, a heteroaromatic compound having a thiazole ring, and a heteroaromatic compound having a benzimidazole ring.

Examples of the heteroaromatic compound having a six-membered ring structure, which is a heteroaromatic compound containing carbon and one or more of nitrogen, oxygen, sulfur, and the like, include a heteroaromatic compound having a heteroaromatic ring, such as a pyridine ring, a diazine ring (including a pyrimidine ring, a pyrazine ring, a pyridazine ring, or the like), a triazine ring, or a polyazole ring. Other examples include a heteroaromatic compound having a bipyridine structure and a heteroaromatic compound having a terpyridine structure, although they are included in examples of a heteroaromatic compound in which pyridine rings are bonded.

Examples of the heteroaromatic compound having a fused ring structure including the above six-membered ring structure as a part include a heteroaromatic compound having a condensed heteroaromatic ring such as a quinoline ring, a benzoquinoline ring, a quinoxaline ring, a dibenzoquinoxaline ring, a phenanthroline ring, a furodiazine ring (including a structure in which an aromatic ring is condensed to a furan ring of a furodiazine ring), or a benzimidazole ring.

Specific examples of the heteroaromatic compound having a five-membered ring structure (e.g., a polyazole ring (including an imidazole ring, a triazole ring, and an oxadiazole ring), an oxazole ring, a thiazole ring, or a benzimidazole ring) include PBD, OXD-7, CO11, TAZ, 3-(4-tert-butylphenyl)-4-(4-ethylphenyl)-5-(4-biphenylyl)-1,2,4-triazole (abbreviation: p-EtTAZ), TPBI, mDBTBIm-II, and BzOS.

Specific examples of the heteroaromatic compound having a six-membered ring structure (including a heteroaromatic ring having a pyridine ring, a diazine ring, a triazine ring, or the like) include a heteroaromatic compound including a heteroaromatic ring having a pyridine ring, such as 35DCzPPy or TmPyPB; a heteroaromatic compound including a heteroaromatic ring having a triazine ring, such as PCCzPTzn, mPCCzPTzn-02, mINc(II)PTzn, mTpBPTzn, BP-SFTzn, 2,4NP-6PyPPm, PCDBfTzn, mBP-TPDBfTzn, 2-{3-[3-(dibenzothiophen-4-yl)phenyl]phenyl}-4,6-diphenyl-1,3,5-triazine (abbreviation: mDBtBPTzn), or mFBPTzn; and a heteroaromatic compound including a heteroaromatic ring having a diazine (pyrimidine) ring, such as 4,6mPnP2Pm, 4,6mDBTP2Pm-II, 4,6mCzP2Pm, 4,6mCzBP2Pm, 6mBP-4Cz2PPm, 6BP-4Cz2PPm, 8-(naphthalen-2-yl)-4-[3-(dibenzothiophen-4-yl)phenyl]-[1]benzofuro[3,2-d]pyrimidine (abbreviation: 8PN-4mDBtPBfpm), 8BP-4mDBtPBfpm, 9mDBtBPNfpr, 9pmDBtBPNfpr, 3,8-bis[3-(dibenzothiophen-4-yl)phenyl]benzofuro[2,3-b]pyrazine (abbreviation: 3,8mDBtP2Bfpr), 4,8-bis[3-(dibenzothiophen-4-yl)phenyl]-[1]benzofuro[3,2-d]pyrimidine (abbreviation: 4,8mDBtP2Bfpm), 8-[3′-(dibenzothiophen-4-yl)biphenyl-3-yl]naphtho[1′,2′: 4,5]furo[3,2-d]pyrimidine (abbreviation: 8mDBtBPNfpm), and 8-[(2,2′-binaphthalen)-6-yl]-4-[3-(dibenzothiophen-4-yl)phenyl]-[1]benzofuro[3,2-d]pyrimidine (abbreviation: 8(pN2)-4mDBtPBfpm). Note that the above aromatic compounds including a heteroaromatic ring include a heteroaromatic compound having a condensed heteroaromatic ring.

Other examples include a heteroaromatic compound including a heteroaromatic ring having a diazine (pyrimidine) ring, such as 2,2′-(pyridine-2,6-diyl)bis(4-phenylbenzo[h]quinazoline) (abbreviation: 2,6(P-Bqn)2Py), 2,2′-(2,2′-bipyridine-6,6′-diyl)bis(4-phenylbenzo[h]quinazoline) (abbreviation: 6,6′(P-Bqn)2BPy), 2,2′-(pyridine-2,6-diyl)bis{4-[4-(2-naphthyl)phenyl]-6-phenylpyrimidine} (abbreviation: 2,6(NP-PPm)2Py), or 6mBP-4Cz2PPm, and a heteroaromatic compound including a heteroaromatic ring having a triazine ring, such as 2,4,6-tris[3′-(pyridin-3-yl)biphenyl-3-yl]-1,3,5-triazine (abbreviation: TmPPPyTz), 2,4,6-tris(2-pyridyl)-1,3,5-triazine (abbreviation: 2Py3Tz), or 2-[3-(2,6-dimethyl-3-pyridyl)-5-(9-phenanthryl)phenyl]-4,6-diphenyl-1,3,5-triazine (abbreviation: mPn-mDMePyPTzn).

Specific examples of the heteroaromatic compound with a fused structure that partly has a six-membered ring structure include heteroaromatic compounds each having a quinoxaline ring, such as BPhen, bathocuproine (abbreviation: BCP), NBPhen, mPPhen2P, 2,6(P-Bqn)2Py, 2mDBTPDBq-II, 2mDBTBPDBq-II, 2mCzBPDBq, 2CzPDBq-III, 7mDBTPDBq-II, 6mDBTPDBq-II, and 2mpPCBPDBq.

For the electron-transport layer, any of the metal complexes given below as well as the heteroaromatic compounds given above can be used. Examples include metal complexes each including a quinoline ring or a benzoquinoline ring, such as tris(8-quinolinolato)aluminum (III) (abbreviation: Alq₃), Almq₃, 8-quinolinolato-lithium (abbreviation: Liq), BeBq2, BAlq, and Znq; and metal complexes each including an oxazole ring or a thiazole ring, such as ZnPBO and ZnBTZ.

It is also possible to use high molecular compounds such as PPy, PF-Py, and PF-BPy as the electron-transport material.

### <Electron-Injection Layer>

The electron-injection layer is a layer containing a substance having a high electron-injection property. The electron-injection layer is a layer for increasing the efficiency of electron injection from the second electrode and is preferably formed using a material whose value of the LUMO level has a small difference (0.5 eV or less) from the work function of a material used for the second electrode. Thus, the electron-injection layer can be formed using an alkali metal, an alkaline earth metal, or a compound thereof, such as lithium, cesium, lithium fluoride (LiF), cesium fluoride (CsF), calcium fluoride (CaF₂), Liq, 2-(2-pyridyl)phenolatolithium (abbreviation: LiPP), 2-(2-pyridyl)-3-pyridinolatolithium (abbreviation: LiPPy), 4-phenyl-2-(2-pyridyl)phenolatolithium (abbreviation: LiPPP), an oxide of lithium (LiOₓ), or cesium carbonate. A rare earth metal such as Yb or a rare earth metal compound such as erbium fluoride (ErF₃) can also be used. To form the electron-injection layer, a plurality of kinds of materials given above may be mixed or stacked. For example, the electron-injection layer may be a stack of layers with different electric resistances. Electride may also be used for the electron-injection layer. Examples of the electride include a substance in which electrons are added at high concentration to calcium oxide-aluminum oxide. Any of the above-described substances used for the electron-transport layer can also be used.

A mixed material in which an organic compound and an electron donor (donor) are mixed may also be used for the electron-injection layer. Such a mixed material is excellent in an electron-injection property and an electron-transport property because electrons are generated in the organic compound by the electron donor. The organic compound here is preferably a material excellent in transporting the generated electrons; specifically, for example, electron-transport materials used for an electron-transport layer described above (e.g., a metal complex and a heteroaromatic compound) can be used. As the electron donor, a substance showing an electron-donating property with respect to an organic compound is used. Specifically, an alkali metal, an alkaline earth metal, and a rare earth metal are preferable, and Li, Cs, Mg, Ca, erbium (Er), Yb, and the like are given. In addition, an alkali metal oxide and an alkaline earth metal oxide are preferable, and lithium oxide, calcium oxide, barium oxide, and the like are given. Alternatively, a Lewis base such as magnesium oxide can be used. Further alternatively, an organic compound such as tetrathiafulvalene (abbreviation: TTF) can be used. Alternatively, a stack of two or more of these materials may be used.

Alternatively, the electron-injection layer may be formed using a mixed material in which an organic compound and a metal are mixed. The organic compound used here preferably has a lowest unoccupied molecular orbital (LUMO) level higher than or equal to −3.6 eV and lower than or equal to −2.3 eV. Moreover, a material having an unshared electron pair is preferable.

Thus, as the organic compound used in the above mixed material, a mixed material obtained by mixing a metal and the heteroaromatic compound given above as the material that can be used for the electron-transport layer may be used. Preferable examples of the heteroaromatic compound include materials having an unshared electron pair, such as a heteroaromatic compound having a five-membered ring structure (e.g., an imidazole ring, a triazole ring, an oxazole ring, an oxadiazole ring, a thiazole ring, or a benzimidazole ring), a heteroaromatic compound having a six-membered ring structure (e.g., a pyridine ring, a diazine ring (including a pyrimidine ring, a pyrazine ring, a pyridazine ring, or the like), a triazine ring, a bipyridine ring, or a terpyridine ring), and a heteroaromatic compound having a fused ring structure including a six-membered ring structure as a part (e.g., a quinoline ring, a benzoquinoline ring, a quinoxaline ring, a dibenzoquinoxaline ring, or a phenanthroline ring). Since the materials are specifically described above, description thereof is omitted here.

As a metal used for the above mixed material, a transition metal that belongs to Group 5, Group 7, Group 9, or Group 11 or a material that belongs to Group 13 in the periodic table is preferably used, and examples include Ag, Cu, Al, and In. Here, the organic compound forms a singly occupied molecular orbital (SOMO) with the transition metal.

For example, in the case where light emitted from the light-emitting layer 113b is amplified in the light-emitting device illustrated in FIG. 3D, the optical path length between the second electrode 102 and the light-emitting layer 113b is preferably less than one fourth of the wavelength λ of light emitted from the light-emitting layer 113b. In this case, the optical path length can be adjusted by changing the thickness of the electron-transport layer 114b or the electron-injection layer 115b.

### <Charge-Generation Layer>

The charge-generation layer has a function of injecting electrons into one of the EL layers and injecting holes into the other of the EL layers when voltage is applied between the first electrode and the second electrode of the light-emitting device having a tandem structure. The charge-generation layer may be either a p-type layer in which an electron acceptor (acceptor) is added to a hole-transport material or an electron-injection buffer layer in which an electron donor (donor) is added to an electron-transport material. Alternatively, both of these layers may be stacked. Furthermore, an electron-relay layer may be provided between the p-type layer and the electron-injection buffer layer. Note that forming the charge-generation layer with the use of any of the above materials can inhibit an increase in driving voltage caused by the stack of the EL layers.

In the case where the charge-generation layer is a p-type layer in which an electron acceptor is added to a hole-transport material, which is an organic compound, any of the materials described in this embodiment can be used as the hole-transport material. Furthermore, F₄-TCNQ, chloranil, and the like can be given as examples of the electron acceptor. Other examples include oxides of metals that belong to Group 4 to Group 8 of the periodic table. Specific examples include vanadium oxide, niobium oxide, tantalum oxide, chromium oxide, molybdenum oxide, tungsten oxide, manganese oxide, and rhenium oxide. Any of the above-described acceptor materials may be used. Furthermore, a mixed film obtained by mixing materials of a p-type layer or a stack of films containing the respective materials may be used.

In the case where the charge-generation layer is an electron-injection buffer layer in which an electron donor is added to an electron-transport material, any of the materials described in this embodiment can be used as the electron-transport material. As the electron donor, it is possible to use an alkali metal, an alkaline earth metal, a rare earth metal, a metal belonging to Group 2 or Group 13 of the periodic table, or an oxide or a carbonate thereof. Specifically, Li, Cs, Mg, calcium (Ca), Yb, In, lithium oxide (Li₂O), cesium carbonate, or the like is preferably used. An organic compound such as tetrathianaphthacene may be used as the electron donor.

When an electron-relay layer is provided between a p-type layer and an electron-injection buffer layer in the charge-generation layer, the electron-relay layer contains at least a substance having an electron-transport property and has a function of preventing an interaction between the electron-injection buffer layer and the p-type layer and transferring electrons smoothly. The LUMO level of the substance having an electron-transport property in the electron-relay layer is preferably between the LUMO level of the acceptor substance in the p-type layer and the LUMO level of the substance having an electron-transport property in the electron-transport layer in contact with the charge-generation layer. Specifically, the LUMO level of the substance having an electron-transport property in the electron-relay layer is preferably higher than or equal to −5.0 eV, further preferably higher than or equal to −5.0 eV and lower than or equal to −3.0 eV. Note that as the substance having an electron-transport property in the electron-relay layer, a phthalocyanine-based material or a metal complex having a metal-oxygen bond and an aromatic ligand is preferably used.

### <Cap Layer>

Although not illustrated in FIGS. 3A to 3E, a cap layer may be provided over the second electrode 102 of the light-emitting device. For example, a material with a high refractive index can be used for the cap layer. When the cap layer is provided over the second electrode 102, extraction efficiency of light emitted from the second electrode 102 can be improved.

Specific examples of a material that can be used for the cap layer include 5,5′-diphenyl-2,2′-di-5H-[1]benzothieno[3,2-c]carbazole (abbreviation: BisBTc) and DBT3P-II.

### <Substrate>

The light-emitting device described in this embodiment can be formed over a variety of substrates. Note that the type of substrate is not limited to a certain type. Examples of the substrate include semiconductor substrates (e.g., a single crystal substrate and a silicon substrate), an SOI substrate, a glass substrate, a quartz substrate, a plastic substrate, a metal substrate, a stainless steel substrate, a substrate including stainless steel foil, a tungsten substrate, a substrate including tungsten foil, a flexible substrate, an attachment film, and paper and a base material film that include a fibrous material.

Examples of the glass substrate include a barium borosilicate glass substrate, an aluminoborosilicate glass substrate, and a soda lime glass substrate. Examples of the flexible substrate, the attachment film, and the base material film include plastics typified by polyethylene terephthalate (PET), polyethylene naphthalate (PEN), and polyether sulfone (PES), a synthetic resin such as an acrylic resin, polypropylene, polyester, polyvinyl fluoride, polyvinyl chloride, polyamide, polyimide, aramid, an epoxy resin, an inorganic vapor deposition film, and paper.

For fabrication of the light-emitting device of this embodiment, a gas phase method such as an evaporation method or a liquid phase method such as a spin coating method or an inkjet method can be used. When an evaporation method is used, a physical vapor deposition method (PVD method) such as a sputtering method, an ion plating method, an ion beam evaporation method, a molecular beam evaporation method, or a vacuum evaporation method, a chemical vapor deposition method (CVD method), or the like can be used. Specifically, the layers having various functions (the hole-injection layer 111, the hole-transport layer 112, the light-emitting layer 113, the electron-transport layer 114, and the electron-injection layer 115) included in the EL layers of the light-emitting device can be formed by an evaporation method (e.g., a vacuum evaporation method), a coating method (e.g., a dip coating method, a die coating method, a bar coating method, a spin coating method, or a spray coating method), a printing method (e.g., an inkjet method, screen printing (stencil), offset printing (planography), flexography (relief printing), gravure printing, or micro-contact printing), or the like.

In the case where a film formation method such as the coating method or the printing method is employed, a high molecular compound (e.g., an oligomer, a dendrimer, or a polymer), a middle molecular compound (a compound between a low molecular compound and a high molecular compound with a molecular weight of 400 to 4000), an inorganic compound (e.g., a quantum dot material), or the like can be used. The quantum dot material can be a colloidal quantum dot material, an alloyed quantum dot material, a core-shell quantum dot material, a core quantum dot material, or the like.

Materials that can be used for the layers (the hole-injection layer 111, the hole-transport layer 112, the light-emitting layer 113, the electron-transport layer 114, and the electron-injection layer 115) included in the EL layer 103 of the light-emitting device described in this embodiment are not limited to the materials described in this embodiment, and other materials can be used in combination as long as the functions of the layers are fulfilled.

Note that in this specification and the like, the terms “layer” and “film” can be interchanged with each other as appropriate.

The structures described in this embodiment can be used in combination with any of the structures described in the other embodiments as appropriate.

### Embodiment 5

This embodiment will describe a light-emitting and light-receiving apparatus 700 as a specific example of a light-emitting apparatus of one embodiment of the present invention and an example of the fabrication method. Note that the light-emitting and light-receiving apparatus 700 includes both a light-emitting device and a light-receiving device, and can also be referred to as a light-emitting apparatus including a light-receiving device or a light-receiving apparatus including a light-emitting device. In addition, the light-emitting and light-receiving apparatus 700 can be used for a display portion of an electronic device or the like, and thus can also be referred to as a display panel or a display device.

### <Structure Example of Light-Emitting and Light-Receiving Apparatus 700>

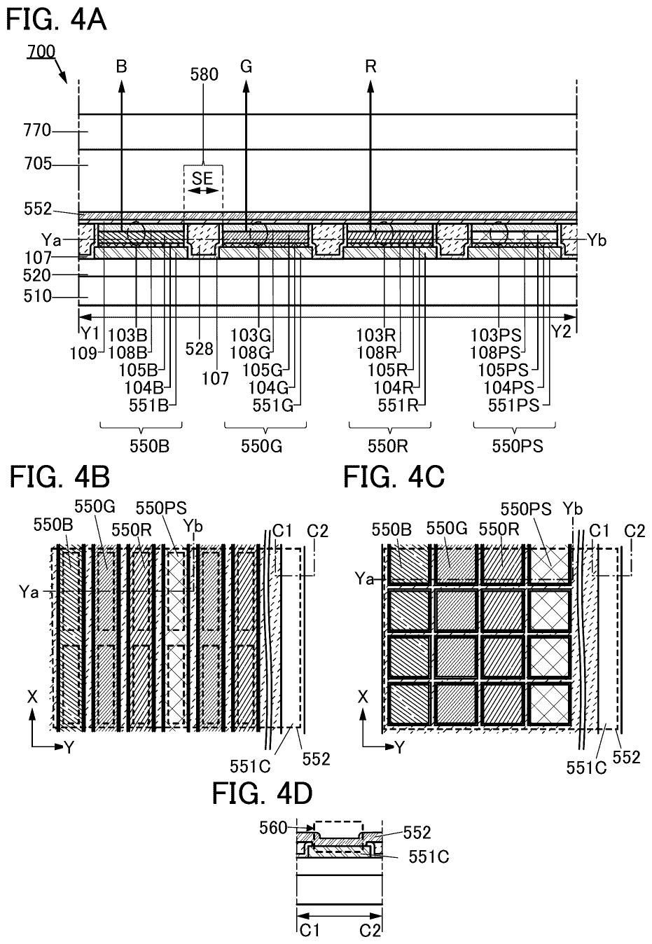

The light-emitting and light-receiving apparatus 700 illustrated in FIG. 4A includes a light-emitting device 550B, a light-emitting device 550G, a light-emitting device 550R, and a light-receiving device 550PS that are formed over a functional layer 520 over a first substrate 510. The functional layer 520 includes, for example, driver circuits such as a gate driver and a source driver that are composed of a plurality of transistors, and wirings that electrically connect these circuits. These driver circuits are electrically connected to the light-emitting device 550B, the light-emitting device 550G, the light-emitting device 550R, and the light-receiving device 550PS, for example, to drive them. The light-emitting and light-receiving apparatus 700 includes an insulating layer 705 over the functional layer 520 and the devices (the light-emitting devices and the light-receiving device), and the insulating layer 705 has a function of attaching a second substrate 770 and the functional layer 520.

At least one of the light-emitting devices 550B, 550G, and 550R has any of the device structures described in Embodiments 2 to 4. Thus, the power consumption of the light-emitting and light-receiving apparatus 700 can be reduced. In addition, the structure of the EL layer 103 (see FIG. 3A) differs between the light-emitting devices; for example, a light-emitting layer 105B of an EL layer 103B can emit blue light, a light-emitting layer 105G of an EL layer 103G can emit green light, and a light-emitting layer 105R of an EL layer 103R can emit red light.

Although the case where the devices (a plurality of light-emitting devices and a light-receiving device) are formed separately is described in this embodiment, part of an EL layer of a light-emitting device (a hole-injection layer, a hole-transport layer, and an electron-transport layer) and part of an active layer of a light-receiving device (the hole-injection layer, the hole-transport layer, and the electron-transport layer) may be formed using the same material at the same time in the fabrication process.

In this specification and the like, a structure in which light-emitting layers in light-emitting devices of different colors (for example, blue (B), green (G), and red (R)) and a light-receiving layer in a light-receiving device are separately formed or separately patterned is sometimes referred to as a side-by-side (SBS) structure. Although the light-emitting device 550B, the light-emitting device 550G, the light-emitting device 550R, and the light-receiving device 550PS are arranged in this order in the light-emitting and light-receiving apparatus 700 illustrated in FIG. 4A, one embodiment of the present invention is not limited to this structure.

In FIG. 4A, the light-emitting device 550B includes an electrode 551i, an electrode 552, and the EL layer 103B interposed between the electrode 551B and the electrode 552. The light-emitting device 550G includes an electrode 551G, the electrode 552, and the EL layer 103G interposed between the electrode 551G and the electrode 552. The light-emitting device 550R includes an electrode 551R, the electrode 552, and the EL layer 103R interposed between the electrode 551R and the electrode 552. The EL layers (103B, 103G, and 103R) each have a stacked-layer structure of layers having different functions including their respective light-emitting layers (105B, 105G, and 105R). Although specific structures of the layers of the light-emitting devices are as described in Embodiments 2 to 4, the present invention is not limited thereto.

In FIG. 4A, the light-receiving device 550PS includes an electrode 551PS, the electrode 552, and a light-receiving layer 103PS provided between the electrode 551PS and the electrode 552. The light-receiving layer 103PS has a stacked-layer structure of layers having different functions including an active layer 105PS. The active layer 105PS contains a semiconductor. Examples of the semiconductor include inorganic semiconductors such as silicon and organic semiconductors such as organic compounds. Specific structures of other layers in the light-receiving device can be similar to the structures of the corresponding layers in the light-emitting device.

FIG. 4A illustrates a case where the EL layer 103B includes a hole-injection/transport layer 104B, the light-emitting layer 105B, an electron-transport layer 108B, and an electron-injection layer 109; the EL layer 103G includes a hole-injection/transport layer 104G, the light-emitting layer 105G, an electron-transport layer 108G, and the electron-injection layer 109; the EL layer 103R includes a hole-injection/transport layer 104R, the light-emitting layer 105R, an electron-transport layer 108R, and the electron-injection layer 109; and the light-receiving layer 103PS includes a hole-injection/transport layer 104PS, the active layer 105PS, an electron-transport layer 108PS, and the electron-injection layer 109. However, the present invention is not limited thereto.

In FIG. 4A, the electron-injection layer 109 and the electrode 552 are layers (common layers) shared by the devices (the light-emitting device 550B, the light-emitting device 550G, the light-emitting device 550R, and the light-receiving device 550PS).

Hereinafter, for simplicity, the light-emitting device 550B, the light-emitting device 550G, and the light-emitting device 550R are collectively referred to as a light-emitting device 550; the electrode 551B, the electrode 551G, and the electrode 551R are collectively referred to as an electrode 551; the EL layer 103B, the EL layer 103G, and the EL layer 103R are collectively referred to as the EL layer 103; the hole-injection/transport layer 104B, the hole-injection/transport layer 104G, and the hole-injection/transport layer 104R are collectively referred to as a hole-injection/transport layer 104; the light-emitting layer 105B, the light-emitting layer 105G, and the light-emitting layer 105R are collectively referred to as a light-emitting layer 105; and the electron-transport layer 108B, the electron-transport layer 108G, and the electron-transport layer 108R are collectively referred to as an electron-transport layer 108, in some cases.

As illustrated in FIG. 4A, an insulating layer 107 may be formed on side surfaces (or end portions) of the hole-injection/transport layer 104, the light-emitting layer 105, and the electron-transport layer 108 included in the EL layer 103, and side surfaces (or end portions) of the hole-injection/transport layer 104PS, the active layer 105PS, and the electron-transport layer 108PS included in the light-receiving layer 103PS. The insulating layer 107 is formed in contact with the side surfaces (or the end portions) of the EL layer 103 and the light-receiving layer 103PS. This can inhibit entry of oxygen, moisture, or constituent elements thereof into the inside through the side surfaces of the EL layer 103 and the light-receiving layer 103PS. Note that the insulating layer 107 continuously covers the side surfaces of part of the EL layer 103 and part of the light-receiving layer 103PS of adjacent devices. For example, in FIG. 4A, the side surfaces of parts of the EL layer 103B of the light-emitting device 550B and the EL layer 103G of the light-emitting device 550G are covered with the continuous insulating layer 107.

As illustrated in FIG. 4A, a partition 528 is provided between the devices. Note that the electron-injection layer 109 and the electrode 552 that are common layers shared by the devices are provided continuously without being divided by the partition 528. Thus, it can be said that the partition 528 is provided in a region surrounded by the electron-injection layer 109 and the insulating layer 107. In addition, the partitions 528 are positioned along side surfaces (or end portions) of the electrode 551, part of the EL layer 103 (the hole-injection/transport layer 104, the light-emitting layer 105, and the electron-transport layer 108), and part of the light-receiving layer 103PS (the hole-injection/transport layer 104, the active layer 105PS, and the electron-transport layer 108) with the insulating layer 107 therebetween.

In each of the EL layer 103 and the light-receiving layer 103PS, particularly the hole-injection layer, which is included in the hole-transport region between the anode and the light-emitting layer and between the anode and the active layer, often has high conductivity; thus, a hole-injection layer formed as a layer shared by adjacent devices might cause crosstalk. Thus, as described in this structure example, part of the EL layer 103 (the hole-injection/transport layer 104, the light-emitting layer 105, and the electron-transport layer 108) and part of the light-receiving layer 103PS (the hole-injection/transport layer 104, the active layer 105PS, and the electron-transport layer 108) are separated, and the insulating layer 107 and the partition 528 are provided therebetween, so that crosstalk between adjacent devices can be inhibited.

Providing the partition 528 can flatten the surface by reducing a depressed portion formed between adjacent devices. When the depressed portion is reduced, disconnection of the electron-injection layer 109 and the electrode 552 formed over the EL layer 103 and the light-receiving layer 103PS can be inhibited.

For the insulating layer 107, aluminum oxide, magnesium oxide, hafnium oxide, gallium oxide, indium gallium zinc oxide, silicon nitride, or silicon nitride oxide can be used, for example. Some of the above-described materials may be stacked to form the insulating layer 107. The insulating layer 107 can be formed by a sputtering method, a CVD method, an MBE method, a PLD method, an ALD method, or the like and is formed preferably by an ALD method, which achieves good coverage.

Examples of an insulating material used to form the partition 528 include organic materials such as an acrylic resin, a polyimide resin, an epoxy resin, an imide resin, a polyamide resin, a polyimide-amide resin, a silicone resin, a siloxane resin, a benzocyclobutene-based resin, a phenol resin, and precursors of these resins. Other examples include organic materials such as polyvinyl alcohol (PVA), polyvinyl butyral, polyvinyl pyrrolidone, polyethylene glycol, polyglycerin, pullulan, water-soluble cellulose, and an alcohol-soluble polyamide resin. A photosensitive resin such as a photoresist can also be used. Examples of the photosensitive resin include positive-type materials and negative-type materials.

With the use of the photosensitive resin, the partition 528 can be formed by only light exposure and developing steps. The partition 528 may be formed using a negative photosensitive resin (e.g., a resist material). In the case where an insulating layer containing an organic material is used as the partition 528, a material absorbing visible light is suitably used. When such a material absorbing visible light is used for the partition 528, light emission from the EL layer can be absorbed by the partition 528, leading to a reduction in light leakage (stray light) to an adjacent EL layer or light-receiving layer. Thus, a light-emitting and light-receiving apparatus having high display quality can be provided.

For example, the difference between the top-surface level of the partition 528 and the top-surface level of the EL layer 103 or the light-receiving layer 103PS is preferably 0.5 times or less, further preferably 0.3 times or less the thickness of the partition 528. The partition 528 may be provided such that the top-surface level of the EL layer 103 or the light-receiving layer 103PS is higher than the top-surface level of the partition 528, for example. Alternatively, the partition 528 may be provided such that the top-surface level of the partition 528 is higher than the top-surface level of the light-emitting layer of the EL layer 103 or the active layer of the light-receiving layer 103PS, for example.

When crosstalk occurs between devices in a light-emitting and light-receiving apparatus with a high resolution exceeding 1000 ppi, a color gamut that the light-emitting and light-receiving apparatus can reproduce is narrowed. In a light-emitting and light-receiving apparatus with a high resolution of 1000 ppi or more, preferably 2000 ppi or more, further preferably 5000 ppi or more, the insulating layer 107 and the partition 528 are provided between part of the EL layer 103 (the hole-injection/transport layer 104, the light-emitting layer 105, and the electron-transport layer 108) and part of the light-receiving layer 103PS (the hole-injection/transport layer 104, the active layer 105PS, and the electron-transport layer 108), whereby the light-emitting and light-receiving apparatus can display bright colors.

FIGS. 4B and 4C are each a schematic top view of the light-emitting and light-receiving apparatus 700 taken along the dashed-dotted line Ya-Yb in the cross-sectional view of FIG. 4A. That is, the devices are arranged in a matrix. Note that FIG. 4B illustrates what is called a stripe arrangement, in which the light-emitting devices of the same color or the light-receiving devices are arranged in the X-direction. FIG. 4C illustrates a structure in which the light-emitting devices of the same color or the light-receiving devices are arranged in the X-direction and separated by patterning for each pixel. Note that the arrangement method of the light-emitting devices is not limited thereto; another method such as a delta, zigzag, PenTile, or diamond arrangement can also be used.

Note that part of the EL layer 103 (the hole-injection/transport layer 104, the light-emitting layer 105, and the electron-transport layer 108) and part of the light-receiving layer 103PS (the hole-injection/transport layer 104, the active layer 105PS, and the electron-transport layer 108) are processed by patterning using a lithography method for separation, so that a light-emitting and light-receiving apparatus (display panel) with a high resolution can be fabricated. End portions (side surfaces) of the layers of the EL layer 103 and the layers of the light-receiving layer 103PS processed by patterning using a photolithography method have substantially one surface (or are positioned on substantially the same plane). In this case, the widths (SE) of spaces 580 between the EL layers and between the EL layer and the light-receiving layer are each preferably 5 μm or less, further preferably 1 μm or less.

FIG. 4D is a schematic cross-sectional view taken along the dashed-dotted line C1-C2 in FIGS. 4B and 4C. FIG. 4D illustrates a connection portion 560 where a connection electrode 551C and the electrode 552 are electrically connected to each other. In the connection portion 560, the electrode 552 is provided over and in contact with the connection electrode 551C. The partition 528 is provided to cover an end portion of the connection electrode 551C.

### <Fabrication Method Example of Light-Emitting and Light-Receiving Apparatus>

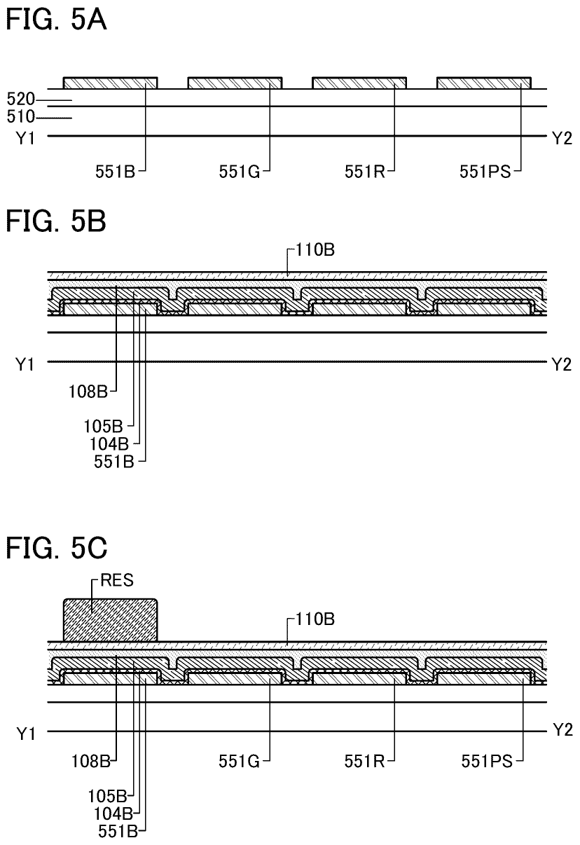

The electrode 551B, the electrode 551G, the electrode 551R, and the electrode 551PS are formed as illustrated in FIG. 5A. For example, a conductive film is formed over the functional layer 520 over the first substrate 510 and processed into predetermined shapes by a photolithography method.

The conductive film can be formed by any of a sputtering method, a chemical vapor deposition (CVD) method, a molecular beam epitaxy (MBE) method, a vacuum evaporation method, a pulsed laser deposition (PLD) method, an atomic layer deposition (ALD) method, and the like. Examples of the CVD method include a plasma-enhanced chemical vapor deposition (PECVD) method and a thermal CVD method. An example of a thermal CVD method is a metal organic CVD (MOCVD) method.

The conductive film may be processed by a nanoimprinting method, a sandblasting method, a lift-off method, or the like as well as a photolithography method described above. Alternatively, island-shaped thin films may be directly formed by a film formation method using a shielding mask such as a metal mask.

There are two typical examples of photolithography methods. In one of the methods, a resist mask is formed over a thin film that is to be processed, the thin film is processed by etching or the like, and then the resist mask is removed. In the other method, a photosensitive thin film is formed and then processed into a desired shape by light exposure and development. The former method involves heat treatment steps such as pre-applied bake (PAB) after resist application and post-exposure bake (PEB) after light exposure. In one embodiment of the present invention, a lithography method is used not only for processing of a conductive film but also for processing of a thin film used for formation of an EL layer (a film made of an organic compound or a film partly including an organic compound).

As light for exposure in a photolithography method, it is possible to use light with the i-line (wavelength: 365 nm), light with the g-line (wavelength: 436 nm), light with the h-line (wavelength: 405 nm), or light in which the i-line, the g-line, and the h-line are mixed. Alternatively, ultraviolet light, KrF laser light, ArF laser light, or the like can be used. Exposure may be performed by liquid immersion exposure technique. As the light for exposure, extreme ultraviolet (EUV) light or X-rays may also be used. Instead of the light for exposure, an electron beam can be used. It is preferable to use EUV, X-rays, or an electron beam because extremely minute processing can be performed. Note that a photomask is not needed when light exposure is performed by scanning with a beam such as an electron beam.

For etching of a thin film using a resist mask, a dry etching method, a wet etching method, a sandblasting method, or the like can be used.

Subsequently, as illustrated in FIG. 5B, the hole-injection/transport layer 104B, the light-emitting layer 105B, and the electron-transport layer 108B are formed over the electrode 551i, the electrode 551G, the electrode 551R, and the electrode 551PS. Note that the hole-injection/transport layer 104B, the light-emitting layer 105B, and the electron-transport layer 108B can be formed by a vacuum evaporation method, for example. Furthermore, a sacrificial layer 110B is formed over the electron-transport layer 108B. For the formation of the hole-injection/transport layer 104B, the light-emitting layer 105B, and the electron-transport layer 108B, any of the materials described in the above embodiments can be used, for example.

For the sacrificial layer 110B, it is preferable to use a film highly resistant to etching treatment performed on the hole-injection/transport layer 104B, the light-emitting layer 105B, and the electron-transport layer 108B, i.e., a film having high etching selectivity with respect to the hole-injection/transport layer 104B, the light-emitting layer 105B, and the electron-transport layer 108B. The sacrificial layer 110B preferably has a stacked-layer structure of a first sacrificial layer and a second sacrificial layer that have different etching selectivities. For the sacrificial layer 110B, it is possible to use a film that can be removed by a wet etching method, which causes less damage to the EL layer 103B. In wet etching, oxalic acid or the like can be used as an etching material. Note that in this specification and the like, a sacrificial layer may be called a mask layer.

For the sacrificial layer 110B, an inorganic film such as a metal film, an alloy film, a metal oxide film, a semiconductor film, or an inorganic insulating film can be used, for example. The sacrificial layer 110B can be formed by any of a variety of film formation methods such as a sputtering method, an evaporation method, a CVD method, and an ALD method.

For the sacrificial layer 110B, a metal material such as gold, silver, platinum, magnesium, nickel, tungsten, chromium, molybdenum, iron, cobalt, copper, palladium, titanium, aluminum, yttrium, zirconium, or tantalum or an alloy material containing the metal material can be used, for example. It is particularly preferable to use a low-melting-point material such as aluminum or silver.

A metal oxide such as indium gallium zinc oxide (also referred to as In—Ga—Zn oxide or IGZO) can be used for the sacrificial layer 110B. It is also possible to use indium oxide, indium zinc oxide (In—Zn oxide), indium tin oxide (In—Sn oxide), indium titanium oxide (In—Ti oxide), indium tin zinc oxide (In—Sn—Zn oxide), indium titanium zinc oxide (In—Ti—Zn oxide), indium gallium tin zinc oxide (In—Ga—Sn—Zn oxide), or the like. Alternatively, indium tin oxide containing silicon can also be used, for example.

An element M (M is one or more of aluminum, silicon, boron, yttrium, copper, vanadium, beryllium, titanium, iron, nickel, germanium, zirconium, molybdenum, lanthanum, cerium, neodymium, hafnium, tantalum, tungsten, and magnesium) may be used instead of gallium. In particular, M is preferably one or more of gallium, aluminum, and yttrium.

For the sacrificial layer 110B, an inorganic insulating material such as aluminum oxide, hafnium oxide, or silicon oxide can be used.

The sacrificial layer 110B is preferably formed using a material that can be dissolved in a solvent chemically stable with respect to at least the electron-transport layer 108B that is in the uppermost position. Specifically, a material that can be dissolved in water or alcohol can be suitably used for the sacrificial layer 110B. In formation of the sacrificial layer 110B, it is preferable that application of such a material dissolved in a solvent such as water or alcohol be performed by a wet process and followed by heat treatment for evaporating the solvent. At this time, the heat treatment is preferably performed under a reduced-pressure atmosphere, in which case the solvent can be removed at a low temperature in a short time and thermal damage to the hole-injection/transport layer 104B, the light-emitting layer 105B, and the electron-transport layer 108B can be accordingly reduced.

In the case where the sacrificial layer 110B having a stacked-layer structure is formed, the stacked-layer structure can include the first sacrificial layer formed using any of the above-described materials and the second sacrificial layer thereover.

The second sacrificial layer in that case is a film used as a hard mask for etching of the first sacrificial layer. In processing the second sacrificial layer, the first sacrificial layer is exposed. Thus, the combination of films having high etching selectivity therebetween is selected for the first sacrificial layer and the second sacrificial layer. Thus, a film that can be used for the second sacrificial layer can be selected in accordance with the etching conditions of the first sacrificial layer and those of the second sacrificial layer.

For example, in the case where the second sacrificial layer is etched by dry etching involving a fluorine-containing gas (also referred to as a fluorine-based gas), the second sacrificial layer can be formed using silicon, silicon nitride, silicon oxide, tungsten, titanium, molybdenum, tantalum, tantalum nitride, an alloy containing molybdenum and niobium, an alloy containing molybdenum and tungsten, or the like. Here, a film of a metal oxide such as IGZO or ITO can be given as an example of a film having a high etching selectivity with respect to the second sacrificial layer (i.e., a film with a low etching rate) in the dry etching involving the fluorine-based gas, and can be used for the first sacrificial layer.

Note that the material for the second sacrificial layer is not limited to the above and can be selected from a variety of materials in accordance with the etching conditions of the first sacrificial layer and those of the second sacrificial layer. For example, any of the films that can be used for the first sacrificial layer can be used for the second sacrificial layer.

For the second sacrificial layer, a nitride film can be used, for example. Specifically, it is possible to use a nitride such as silicon nitride, aluminum nitride, hafnium nitride, titanium nitride, tantalum nitride, tungsten nitride, gallium nitride, or germanium nitride.

Alternatively, an oxide film can be used for the second sacrificial layer. Typically, it is possible to use a film of an oxide or an oxynitride such as silicon oxide, silicon oxynitride, aluminum oxide, aluminum oxynitride, hafnium oxide, or hafnium oxynitride.

Next, as illustrated in FIG. 5C, a resist is applied onto the sacrificial layer 110B, and the resist having a desired shape (a resist mask RES) is formed by a photolithography method. Such a method involves heat treatment steps such as pre-applied bake (PAB) after the resist application and post-exposure bake (PEB) after light exposure. The temperature reaches approximately 100° C. during the PAB, and approximately 120° C. during the PEB, for example. Therefore, the light-emitting device should be resistant to such high treatment temperatures.

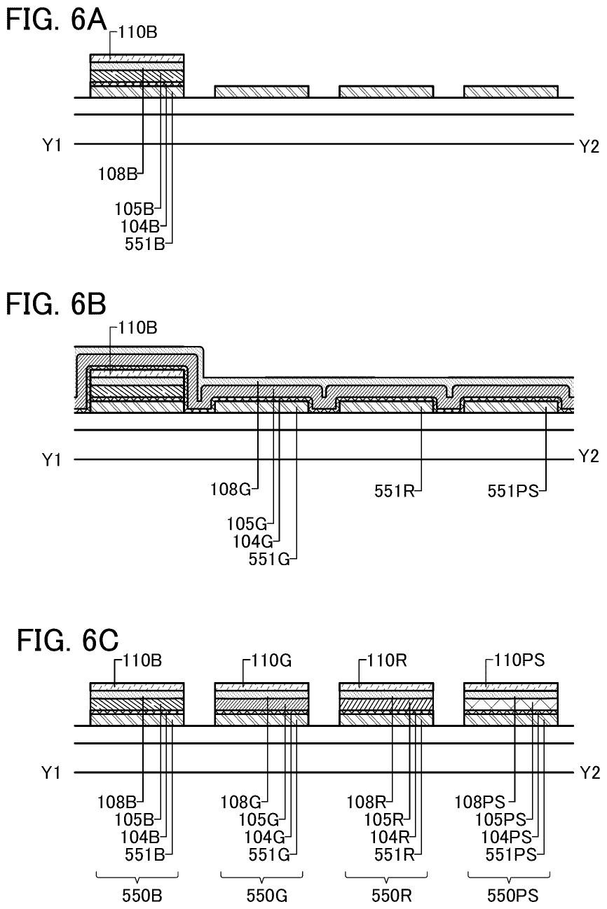

Next, part of the sacrificial layer 110B that is not covered with the resist mask RES is removed by etching using the resist mask RES, the resist mask RES is removed, and then the hole-injection/transport layer 104B, the light-emitting layer 105B, and the electron-transport layer 108B that are not covered with the sacrificial layer 110B are removed by etching, so that the hole-injection/transport layer 104B, the light-emitting layer 105B, and the electron-transport layer 108B are processed to have side surfaces (or have their side surfaces exposed) over the electrode 551B or have belt-like shapes extending in the direction intersecting the sheet of the diagram. Note that dry etching is preferably employed for the etching. In the case where the sacrificial layer 110B has the aforementioned stacked-layer structure of the first sacrificial layer and the second sacrificial layer, the hole-injection/transport layer 104B, the light-emitting layer 105B, and the electron-transport layer 108B may be processed into a predetermined shape in the following manner: part of the second sacrificial layer is etched using the resist mask RES, the resist mask RES is then removed, and part of the first sacrificial layer is etched using the second sacrificial layer as a mask. The structure illustrated in FIG. 6A is obtained through these etching steps.

Subsequently, as illustrated in FIG. 6B, the hole-injection/transport layer 104G, the light-emitting layer 105G, and the electron-transport layer 108G are formed over the sacrificial layer 110B, the electrode 551G, the electrode 551R, and the electrode 551PS. The hole-injection/transport layer 104G, the light-emitting layer 105G, and the electron-transport layer 108G can be formed using any of the materials described in the above embodiments, for example. Note that the hole-injection/transport layer 104G, the light-emitting layer 105G, and the electron-transport layer 108G can be formed by a vacuum evaporation method, for example.

Hereinafter, in a manner similar to formation of the hole-injection/transport layer 104B, the light-emitting layer 105B, the electron-transport layer 108B, and the sacrificial layer 110B, the hole-injection/transport layer 104G, the light-emitting layer 105G, the electron-transport layer 108G, and a sacrificial layer 110G are formed over the electrode 551G, the hole-injection/transport layer 104R, the light-emitting layer 105R, the electron-transport layer 108R, and a sacrificial layer 110R are formed over the electrode 551R, and the hole-injection/transport layer 104PS, the active layer 105PS, the electron-transport layer 108PS, and a sacrificial layer 110PS are formed over the electrode 551PS, whereby the structure illustrated in FIG. 6C is obtained.

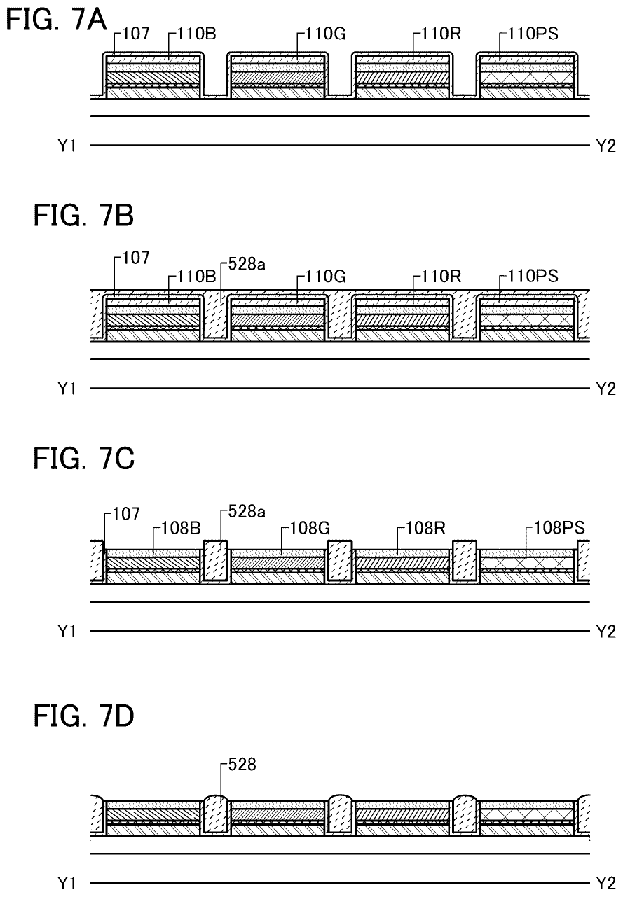

Next, as illustrated in FIG. 7A, the insulating layer 107 is formed over the sacrificial layers 110B, 110G, 110R, and 110PS.

Note that the insulating layer 107 can be formed by an ALD method, for example. In this case, as illustrated in FIG. 7A, the insulating layer 107 is formed to be in contact with the side surfaces (end portions) of the hole-injection/transport layers (104B, 104G, 104R, and 104PS), the light-emitting layers (105B, 105G, and 105R), the active layer 105PS, and the electron-transport layers (108B, 108G, 108R, and 108PS) of the devices. This can inhibit entry of oxygen, moisture, or constituent elements thereof into the inside through the side surfaces of the layers.

Next, as illustrated in FIG. 7B, a resin film 528a is formed over the insulating layer 107. As the resin film 528a, for example, a negative photosensitive resin or a positive photosensitive resin can be used.

Then, as illustrated in FIG. 7C, part of the resin film 528a, part of the insulating layer 107, and the sacrificial layers (110B, 110G, 110R, and 110PS) are removed to expose the top surfaces of the electron-transport layers (108B, 108G, 108R, and 108PS).

Next, heat treatment is performed to process an upper edge portion of the resin film 528a into a curved shape, so that the partition 528 is formed, as illustrated in FIG. 7D. When the upper edge portion of the partition 528 has a curved shape, good coverage with the electron-injection layer 109 to be formed later can be obtained. For example, in the case of using a positive photosensitive acrylic resin as a material for the resin film 528a, the partition 528 is preferably formed so as to have a curved surface with a curvature radius (0.2 μm to 3 μm) at the upper edge portion.

Next, the electron-injection layer 109 is formed over the insulating layer 107, the electron-transport layers (108B, 108G, 108R, and 108PS), and the partition 528. The electron-injection layer 109 can be formed using any of the materials described in the above embodiments. The electron-injection layer 109 is formed by a vacuum evaporation method, for example.

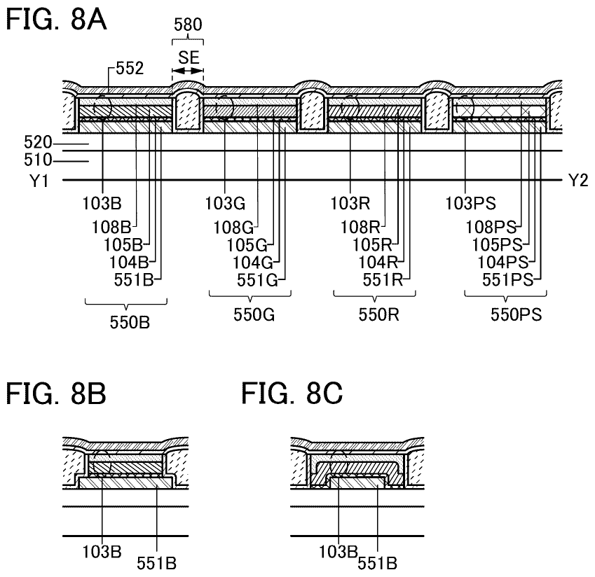

Next, as illustrated in FIG. 8A, the electrode 552 is formed over the electron-injection layer 109. The electrode 552 is formed by a vacuum evaporation method, for example.

Through the above steps, the EL layer 103B, the EL layer 103G, the EL layer 103R, and the light-receiving layer 103PS in the light-emitting device 550B, the light-emitting device 550G, the light-emitting device 550R, and the light-receiving device 550PS can be processed to be separated from each other.

Pattern formation by a photolithography method is performed in separate processing of the EL layer 103 and the light-receiving layer 103PS in the above manner, so that a light-emitting and light-receiving apparatus (display panel) with a high resolution can be fabricated. End portions (side surfaces) of the layers of the EL layer and the light-receiving layer processed by patterning using a photolithography method have substantially one surface (or are positioned on substantially the same plane). The pattern formation by a photolithography method can inhibit crosstalk between adjacent light-emitting devices and between the light-emitting device and the light-receiving device. In addition, the space 580 is provided between adjacent devices processed by patterning using a photolithography method. In FIG. 8C, when the space 580 is denoted by a distance SE between the EL layers of adjacent light-emitting devices, decreasing the distance SE can increase the aperture ratio and the resolution. By contrast, as the distance SE is increased, the effect of the difference in the fabrication process between the adjacent light-emitting devices becomes permissible, which leads to an increase in manufacturing yield. Since the light-emitting device fabricated according to this specification is suitable for a miniaturization process, the distance SE between the EL layers of adjacent light-emitting devices can be longer than or equal to 0.5 μm and shorter than or equal to 5 μm, preferably longer than or equal to 1 μm and shorter than or equal to 3 μm, further preferably longer than or equal to 1 μm and shorter than or equal to 2.5 μm, still further preferably longer than or equal to 1 μm and shorter than or equal to 2 μm. Typically, the distance SE is preferably longer than or equal to 1 μm and shorter than or equal to 2 μm (e.g., 1.5 μm or a neighborhood thereof).

In this specification and the like, a device formed using a metal mask or a fine metal mask (FMM) is sometimes referred to as a device having a metal mask (MM) structure. In this specification and the like, a device formed without using a metal mask or an FMM is sometimes referred to as a device having a metal maskless (MML) structure. Since a light-emitting and light-receiving apparatus having the MML structure is formed without using a metal mask, the pixel arrangement, the pixel shape, and the like can be designed more flexibly than in a light-emitting and light-receiving apparatus having the FMM structure or the MM structure.

Note that the island-shaped EL layers of the light-emitting and light-receiving apparatus having the MML structure are formed by not patterning using a metal mask but processing after formation of an EL layer. Thus, a light-emitting and light-receiving apparatus with a higher resolution or a higher aperture ratio than a conventional one can be achieved. Moreover, EL layers can be formed separately for each color, which enables extremely clear images; thus, a light-emitting and light-receiving apparatus with a high contrast and high display quality can be achieved. Furthermore, provision of a sacrificial layer over an EL layer can reduce damage on the EL layer during the fabrication process and increase the reliability of the light-emitting device.

In FIG. 4A and FIG. 8A, the width of the EL layer 103 is substantially equal to that of the electrode 551 in the light-emitting device 550, and the width of the light-receiving layer 103PS is substantially equal to that of the electrode 551PS in the light-receiving device 550PS; however, one embodiment of the present invention is not limited thereto.

In the light-emitting device 550, the width of the EL layer 103 may be smaller than that of the electrode 551. In the light-receiving device 550PS, the width of the light-receiving layer 103PS may be smaller than that of the electrode 551PS. FIG. 8B illustrates an example in which the width of the EL layer 103B is smaller than that of the electrode 551B in the light-emitting device 550B.

In the light-emitting device 550, the width of the EL layer 103 may be larger than that of the electrode 551. In the light-receiving device 550PS, the width of the light-receiving layer 103PS may be larger than that of the electrode 551PS. FIG. 8C illustrates an example in which the width of the EL layer 103R is larger than that of the electrode 551R in the light-emitting device 550R.

The structures described in this embodiment can be used in combination with any of the structures described in the other embodiments as appropriate.

### Embodiment 6

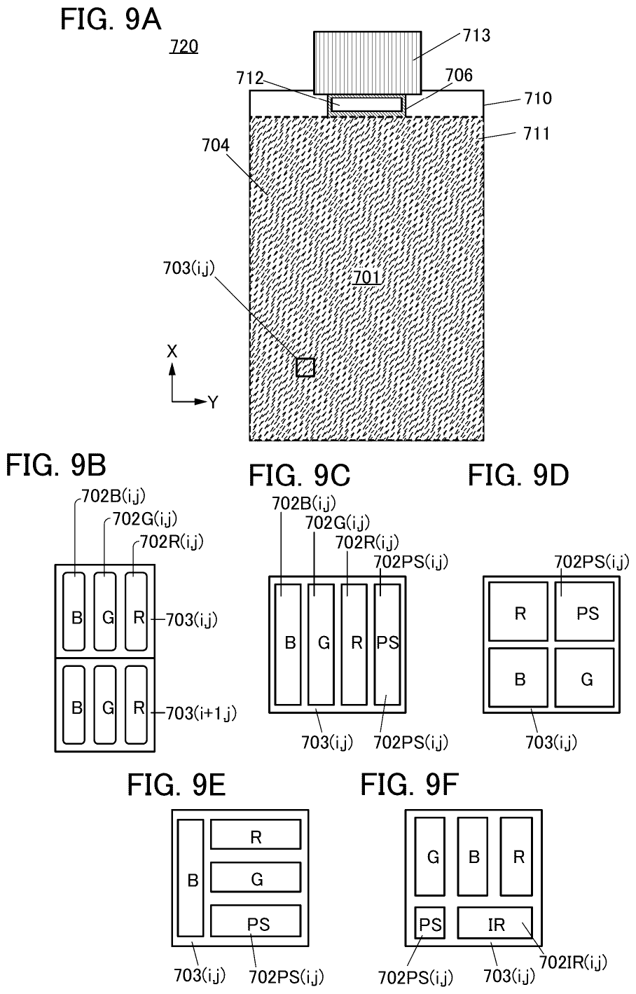

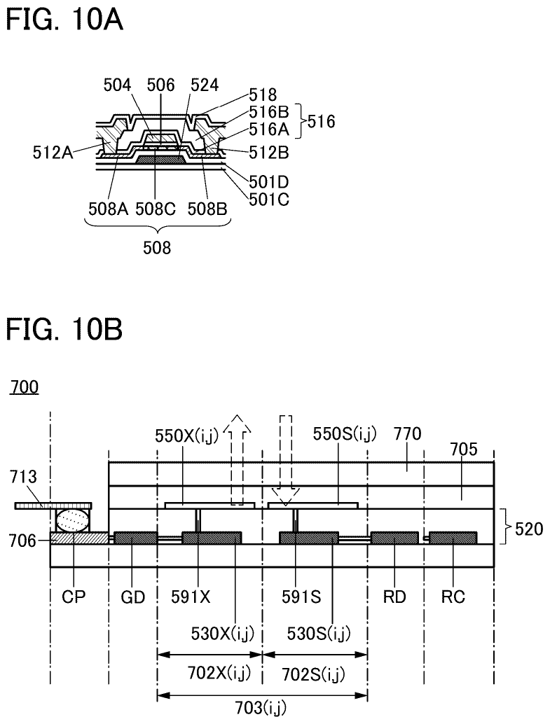

In this embodiment, an apparatus 720 is described with reference to FIGS. 9A to 9F and FIGS. 10A and 10B. The apparatus 720 illustrated in FIGS. 9A to 9F and FIGS. 10A and 10B includes a light-emitting device and thus is a light-emitting apparatus. Furthermore, the apparatus 720 can be used in a display portion of an electronic device or the like and thus can also be referred to as a display panel or a display device. Moreover, when the apparatus 720 includes the light-emitting device as a light source and a light-receiving device that can receive light from the light-emitting device, the apparatus 720 can be referred to as a light-emitting and light-receiving apparatus. Note that the light-emitting apparatus, the display panel, the display device, and the light-emitting and light-receiving apparatus each include at least a light-emitting device.

Furthermore, the light-emitting apparatus, the display panel, the display device, and the light-emitting and light-receiving apparatus of this embodiment can each have high definition or a large size. Therefore, the light-emitting apparatus, the display panel, the display device, and the light-emitting and light-receiving apparatus of this embodiment can be used, for example, in display portions of electronic devices such as a digital camera, a digital video camera, a digital photo frame, a mobile phone, a portable game console, a smartphone, a wristwatch terminal, a tablet terminal, a portable information terminal, and an audio reproducing device, in addition to display portions of electronic devices with a relatively large screen, such as a television device, a desktop or laptop personal computer, a monitor of a computer or the like, digital signage, and a large game machine such as a pachinko machine.

FIG. 9A is a top view of the apparatus 720.

In FIG. 9A, the apparatus 720 has a structure in which a substrate 710 and a substrate 711 are attached to each other. In addition, the apparatus 720 includes a display region 701, a circuit 704, a wiring 706, and the like. Note that the display region 701 includes a plurality of pixels. As illustrated in FIG. 9B, a pixel 703(i,j) illustrated in FIG. 9A and a pixel 703(i+1, j) are adjacent to each other.

Furthermore, in the example of the apparatus 720 illustrated in FIG. 9A, the substrate 710 is provided with an integrated circuit (IC) 712 by a chip on glass (COG) method, a chip on film (COF) method, or the like. As the IC 712, an IC including a scan line driver circuit, a signal line driver circuit, or the like can be used, for example. In the example illustrated in FIG. 9A, an IC including a signal line driver circuit is used as the IC 712, and a scan line driver circuit is used as the circuit 704.

The wiring 706 has a function of supplying signals and power to the display region 701 and the circuit 704. The signals and power are input to the wiring 706 from the outside through a flexible printed circuit (FPC) 713 or to the wiring 706 from the IC 712. Note that the apparatus 720 is not necessarily provided with the IC. The IC may be mounted on the FPC by a COF method or the like.

FIG. 9B illustrates the pixel 703(i, j) and the pixel 703(i+1, j) of the display region 701. A plurality of kinds of subpixels including light-emitting devices that emit light of different colors can be included in the pixel 703(i, j). Alternatively, a plurality of subpixels including light-emitting devices that emit light of the same color may be included in addition to the above-described subpixels. For example, three kinds of subpixels can be included. The three subpixels can be of three colors of red (R), green (G), and blue (B) or of three colors of yellow (Y), cyan (C), and magenta (M), for example. Alternatively, the pixel can include four kinds of subpixels. The four subpixels can be of four colors of R, G, B, and white (W) or of four colors of R, G, B, and Y, for example. Specifically, the pixel 703(i, j) can consist of a subpixel 702B(i, j) for blue display, a subpixel 702G(i, j) for green display, and a subpixel 702R(i, j) for red display.

Other than the subpixels including the light-emitting devices, a subpixel including a light-receiving device may also be provided. In the case where the subpixel includes a light-receiving device, the apparatus 720 is also referred to as a light-emitting and light-receiving apparatus.

FIGS. 9C to 9F illustrate various layout examples of the pixel 703(i,j) including a subpixel 702PS(i,j) including a light-receiving device. The pixel arrangement in FIG. 9C is stripe arrangement, and the pixel arrangement in FIG. 9D is matrix arrangement. The pixel arrangement in FIG. 9E has a structure in which three subpixels (the subpixels R, G, and PS) are vertically arranged next to one subpixel (the subpixel B).

Furthermore, as illustrated in FIG. 9F, a subpixel 702IR(i, j) that emits infrared light may be added to any of the above-described sets of subpixels in the pixel 703(i, j). In the pixel arrangement in FIG. 9F, the vertically oriented three subpixels G, B, and R are arranged laterally, and the subpixel PS and the horizontally oriented subpixel IR are arranged laterally below the three subpixels. Specifically, the subpixel 702IR(i, j) that emits light including light with a wavelength ranging from 650 nm to 1000 nm, inclusive, may be used in the pixel 703(i, j). Note that the wavelength of light detected by the subpixel 702PS(i, j) is not particularly limited; however, the light-receiving device included in the subpixel 702PS(i, j) preferably has sensitivity to light emitted from the light-emitting device included in the subpixel 702R(i, j), the subpixel 702G(i, j), the subpixel 702B(i, j), or the subpixel 702IR(i, j). For example, the light-receiving device preferably detects one or more kinds of light in blue, violet, bluish violet, green, yellowish green, yellow, orange, red, and infrared wavelength ranges, for example.

Note that the arrangement of subpixels is not limited to the structures illustrated in FIGS. 9B to 9F and a variety of arrangement methods can be employed. The arrangement of subpixels may be stripe arrangement, S stripe arrangement, matrix arrangement, delta arrangement, Bayer arrangement, or PenTile arrangement, for example.

Furthermore, top surfaces of the subpixels may have a triangular shape, a quadrangular shape (including a rectangular shape and a square shape), a polygonal shape such as a pentagonal shape, a polygonal shape with rounded corners, an elliptical shape, or a circular shape, for example. The top surface shape of a subpixel herein refers to a top surface shape of a light-emitting region of a light-emitting device.

In the case where not only a light-emitting device but also a light-receiving device is included in a pixel, the pixel has a light-receiving function and thus can detect a contact or approach of an object while displaying an image. For example, an image can be displayed by using all the subpixels included in a light-emitting apparatus; or light can be emitted from some of the subpixels as a light source and an image can be displayed by using the remaining subpixels.

Note that the light-receiving area of the subpixel 702PS(i, j) is preferably smaller than the light-emitting areas of the other subpixels. A smaller light-receiving area leads to a narrower image-capturing range, inhibits a blur in a captured image, and improves the definition. Thus, by using the subpixel 702PS(i, j), high-resolution or high-definition image capturing is possible. For example, image capturing for personal authentication with the use of a fingerprint, a palm print, the iris, the shape of a blood vessel (including the shape of a vein and the shape of an artery), a face, or the like is possible by using the subpixel 702PS(i, j).

Moreover, the subpixel 702PS(i, j) can be used in a touch sensor (also referred to as a direct touch sensor), a near touch sensor (also referred to as a hover sensor, a hover touch sensor, a contactless sensor, or a touchless sensor), or the like. For example, the subpixel 702PS(i, j) preferably detects infrared light. Thus, touch sensing is possible even in a dark place.

Here, the touch sensor or the near touch sensor can detect an approach or contact of an object (e.g., a finger, a hand, or a pen). The touch sensor can detect the object when the light-emitting and light-receiving apparatus and the object come in direct contact with each other. Furthermore, the near touch sensor can detect the object even when the object is not in contact with the light-emitting and light-receiving apparatus. For example, the light-emitting and light-receiving apparatus can preferably detect the object when the distance between the light-emitting and light-receiving apparatus and the object is more than or equal to 0.1 mm and less than or equal to 300 mm, further preferably more than or equal to 3 mm and less than or equal to 50 mm. With this structure, the light-emitting and light-receiving apparatus can be controlled without the object directly contacting with the light-emitting and light-receiving apparatus. In other words, the light-emitting and light-receiving apparatus can be controlled in a contactless (touchless) manner. With the above-described structure, the light-emitting and light-receiving apparatus can be controlled with a reduced risk of being dirty or damaged, or without direct contact between the object and a dirt (e.g., dust, bacteria, or a virus) attached to the light-emitting and light-receiving apparatus.

In the case where the subpixel 702PS(i, j) is used for high-resolution image capturing, the subpixel 702PS(i,j) is preferably provided in every pixel. Meanwhile, in the case where the subpixel 702PS(i,j) is used in a touch sensor, a near touch sensor, or the like, high accuracy is not required as compared to the case of capturing an image of a fingerprint or the like; accordingly, the subpixel 702PS(i,j) is provided in some subpixels. When the number of subpixels 702PS(i,j) is smaller than the number of subpixels 702R(i, j) or the like, higher detection speed can be achieved.

FIG. 10A illustrates an example of a specific structure of a transistor that can be used in the pixel circuit of the subpixel including the light-emitting device. As the transistor, a bottom-gate transistor, a top-gate transistor, or the like can be used as appropriate.

The transistor illustrated in FIG. 10A includes a semiconductor film 508, a conductive film 504, an insulating film 506, a conductive film 512A, and a conductive film 512B. The transistor is formed over an insulating film 501C, for example. The transistor also includes an insulating film 516 (an insulating film 516A and an insulating film 516B) and an insulating film 518.

The semiconductor film 508 includes a region 508A electrically connected to the conductive film 512A and a region 508B electrically connected to the conductive film 512B. The semiconductor film 508 includes a region 508C between the region 508A and the region 508B.

The conductive film 504 includes a region overlapping with the region 508C and has a function of a gate electrode.

The insulating film 506 includes a region interposed between the semiconductor film 508 and the conductive film 504. The insulating film 506 has a function of a first gate insulating film.

The conductive film 512A has one of a function of a source electrode and a function of a drain electrode, and the conductive film 512B has the other thereof.

A conductive film 524 can be used in the transistor. The semiconductor film 508 is interposed between the conductive film 504 and a region included in the conductive film 524. The conductive film 524 has a function of a second gate electrode. An insulating film 501D is interposed between the semiconductor film 508 and the conductive film 524 and has a function of a second gate insulating film.

The insulating film 516 functions as, for example, a protective film covering the semiconductor film 508. Specifically, a film including a silicon oxide film, a silicon oxynitride film, a silicon nitride oxide film, a silicon nitride film, an aluminum oxide film, a hafnium oxide film, an yttrium oxide film, a zirconium oxide film, a gallium oxide film, a tantalum oxide film, a magnesium oxide film, a lanthanum oxide film, a cerium oxide film, or a neodymium oxide film can be used as the insulating film 516, for example.

For the insulating film 518, a material that has a function of inhibiting diffusion of oxygen, hydrogen, water, an alkali metal, an alkaline earth metal, and the like is preferably used. Specifically, the insulating film 518 can be formed using silicon nitride, silicon oxynitride, aluminum nitride, or aluminum oxynitride, for example. In each of silicon oxynitride and aluminum oxynitride, the number of nitrogen atoms contained is preferably larger than the number of oxygen atoms contained.

Note that in a step of forming the semiconductor film used in the transistor of the pixel circuit, the semiconductor film used in the transistor of the driver circuit can be formed. A semiconductor film having the same composition as the semiconductor film used in the transistor of the pixel circuit can be used in the driver circuit, for example.

The semiconductor film 508 preferably contains indium, M (M is one or more of gallium, aluminum, silicon, boron, yttrium, tin, copper, vanadium, beryllium, titanium, iron, nickel, germanium, zirconium, molybdenum, lanthanum, cerium, neodymium, hafnium, tantalum, tungsten, and magnesium), and zinc, for example. Specifically, M is preferably one or more of aluminum, gallium, yttrium, and tin.

In particular, an oxide containing In, Ga, and Zn (also referred to as IGZO) is preferably used as the semiconductor film 508. Alternatively, it is preferable to use an oxide containing In, Sn, and Zn. Further alternatively, it is preferable to use an oxide containing In, Ga, Sn, and Zn. Further alternatively, it is preferable to use an oxide containing In, Al, and Zn (also referred to as IAZO). Further alternatively, it is preferable to use an oxide containing In, Al, Ga, and Zn (also referred to as IAGZO).

When the semiconductor film is an In-M-Zn oxide, the atomic proportion of In is preferably greater than or equal to the atomic proportion of M in the In-M-Zn oxide. Examples of the atomic ratio of the metal elements in such an In-M-Zn oxide include In:M:Zn=1:1:1, 1:1:1.2, 1:3:2, 1:3:4, 2:1:3, 3:1:2, 4:2:3, 4:2:4.1, 5:1:3, 5:1:6, 5:1:7, 5:1:8, 6:1:6, and 5:2:5 and a composition in the vicinity of any of the above atomic ratios. Note that the vicinity of the atomic ratio includes ±30% of an intended atomic ratio.

For example, when the atomic ratio is described as In:Ga:Zn=4:2:3 or a composition in the vicinity thereof, the case is included where the atomic proportion of Ga is greater than or equal to 1 and less than or equal to 3 and the atomic proportion of Zn is greater than or equal to 2 and less than or equal to 4 with the atomic proportion of In being 4. In addition, when the atomic ratio is described as In:Ga:Zn=5:1:6 or a composition in the vicinity thereof, the case is included where the atomic proportion of Ga is greater than 0.1 and less than or equal to 2 and the atomic proportion of Zn is greater than or equal to 5 and less than or equal to 7 with the atomic proportion of In being 5. Furthermore, when the atomic ratio is described as In:Ga:Zn=1:1:1 or a composition in the vicinity thereof, the case is included where the atomic proportion of Ga is greater than 0.1 and less than or equal to 2 and the atomic proportion of Zn is greater than 0.1 and less than or equal to 2 with the atomic proportion of In being 1.

There is no particular limitation on the crystallinity of a semiconductor material used in the transistor, and an amorphous semiconductor or a semiconductor having crystallinity (a microcrystalline semiconductor, a polycrystalline semiconductor, a single crystal semiconductor, or a semiconductor partly including crystal regions) can be used. It is preferable to use a semiconductor having crystallinity, in which case degradation of transistor characteristics can be inhibited.

In the case of using a metal oxide for the semiconductor film 508, the apparatus 720 includes a light-emitting device including a metal oxide in its semiconductor film and having a metal maskless (MML) structure. With this structure, leakage current that might flow through the transistor and leakage current that might flow between adjacent light-emitting devices (also referred to as lateral leakage current, side leakage current, or the like) can be extremely low. With the structure, a viewer can observe any one or more of the image crispness, the image sharpness, a high chroma, and a high contrast ratio in an image displayed on the display device. When the leakage current that might flow through the transistor and the lateral leakage current that might flow between light-emitting devices are extremely low, display with little leakage of light at the time of black display (what is called black floating) (such display is also referred to as deep black display) can be achieved.

Alternatively, silicon may be used for the semiconductor film 508. Examples of silicon include single crystal silicon, polycrystalline silicon, and amorphous silicon. In particular, a transistor containing low-temperature polysilicon (LTPS) in its semiconductor layer (hereinafter, also referred to as an LTPS transistor) is preferably used. The LTPS transistor has high field-effect mobility and excellent frequency characteristics.

With the use of transistors containing silicon such as LTPS transistors, a circuit required to be driven at a high frequency (e.g., a source driver circuit) can be formed on the same substrate as the display portion. This allows simplification of an external circuit mounted on the light-emitting apparatus and reductions in component costs and component-mounting costs.

The structure of the transistors used in the display panel may be selected as appropriate depending on the size of the screen of the display panel. For example, single crystal Si transistors can be used in the display panel with a screen diagonal greater than or equal to 0.1 inches and less than or equal to 3 inches. In addition, LTPS transistors can be used in the display panel with a screen diagonal greater than or equal to 0.1 inches and less than or equal to 30 inches, preferably greater than or equal to 1 inch and less than or equal to 30 inches. In addition, an LTPO structure (where an LTPS transistor and an OS transistor are used in combination) can be used in the display panel with a screen diagonal greater than or equal to 0.1 inches and less than or equal to 50 inches, preferably greater than or equal to 1 inch and less than or equal to 50 inches. In addition, OS transistors (transistors including a metal oxide in a semiconductor where a channel is formed) can be used in the display panel with a screen diagonal greater than or equal to 0.1 inches and less than or equal to 200 inches, preferably greater than or equal to 50 inches and less than or equal to 100 inches.

With the use of single crystal Si transistors, an increase in screen size is extremely difficult due to the size of a single crystal Si substrate. Furthermore, since a laser crystallization apparatus is used in the manufacturing process, LTPS transistors are unlikely to respond to an increase in screen size (typically to a screen diagonal greater than 30 inches). By contrast, since the manufacturing process does not necessarily require a laser crystallization apparatus or the like or can be performed at a relatively low temperature (typically, lower than or equal to 450° C.), OS transistors can be used for a display panel with a relatively large area (typically, a screen diagonal greater than or equal to 50 inches and less than or equal to 100 inches). In addition, LTPO can be used for a display panel with a size (typically, a screen diagonal greater than or equal to 1 inch and less than or equal to 50 inches) midway between the structure using LTPS transistors and the structure using OS transistors.

Next, a cross-sectional view of a light-emitting and light-receiving apparatus is shown. FIG. 10B is a cross-sectional view of the light-emitting and light-receiving apparatus illustrated in FIG. 9A.

FIG. 10B is a cross-sectional view of part of a region including the FPC 713 and the wiring 706 and part of the display region 701 including the pixel 703(i, j).

In FIG. 10B, the light-emitting and light-receiving apparatus 700 includes the functional layer 520 between the first substrate 510 and the second substrate 770. The functional layer 520 includes, as well as the above-described transistors, the capacitors, and the like, wirings electrically connected to these components, for example. Although the functional layer 520 includes a pixel circuit 530X(i, j), a pixel circuit 530S(i, j), and a circuit GD in FIG. 10B, one embodiment of the present invention is not limited thereto.

Furthermore, each pixel circuit (e.g., the pixel circuit 530X(i, j) and the pixel circuit 530S(i, j) in FIG. 10B) included in the functional layer 520 is electrically connected to a light-emitting device and a light-receiving device (e.g., a light-emitting device 550X(i, j) and a light-receiving device 550S(i, j) in FIG. 10B) formed over the functional layer 520. Specifically, the light-emitting device 550X(i, j) is electrically connected to the pixel circuit 530X(i, j) through a wiring 591X, and the light-receiving device 550S(i, j) is electrically connected to the pixel circuit 530S(i, j) through a wiring 591S. The insulating layer 705 is provided over the functional layer 520, the light-emitting devices, and the light-receiving device, and has a function of attaching the second substrate 770 and the functional layer 520.

As the second substrate 770, a substrate where touch sensors are arranged in a matrix can be used. For example, a substrate provided with capacitive touch sensors or optical touch sensors can be used as the second substrate 770. Thus, the light-emitting and light-receiving apparatus of one embodiment of the present invention can be used as a touch panel.

The structures described in this embodiment can be used in appropriate combination with any of the structures described in the other embodiments.

### Embodiment 7

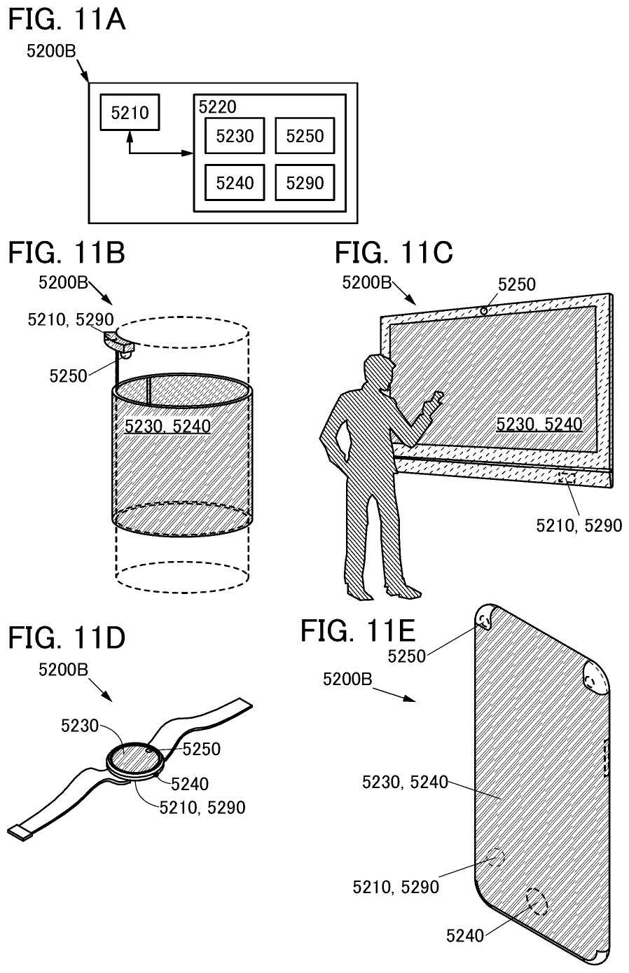

This embodiment will describe structures of electronic devices of embodiments of the present invention with reference to FIGS. 11A to 11E, FIGS. 12A to 12E, and FIGS. 13A and 13B.

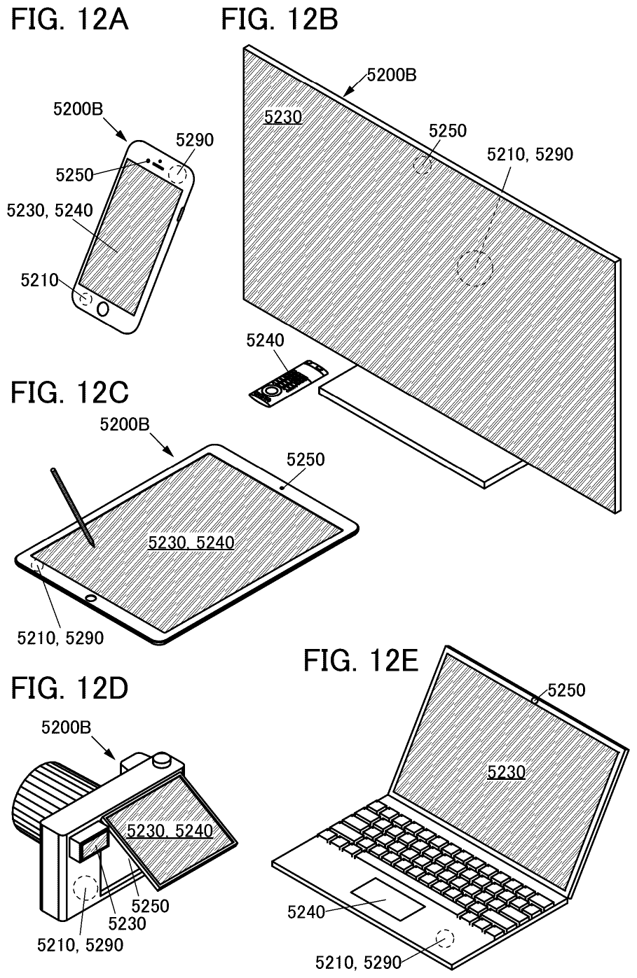

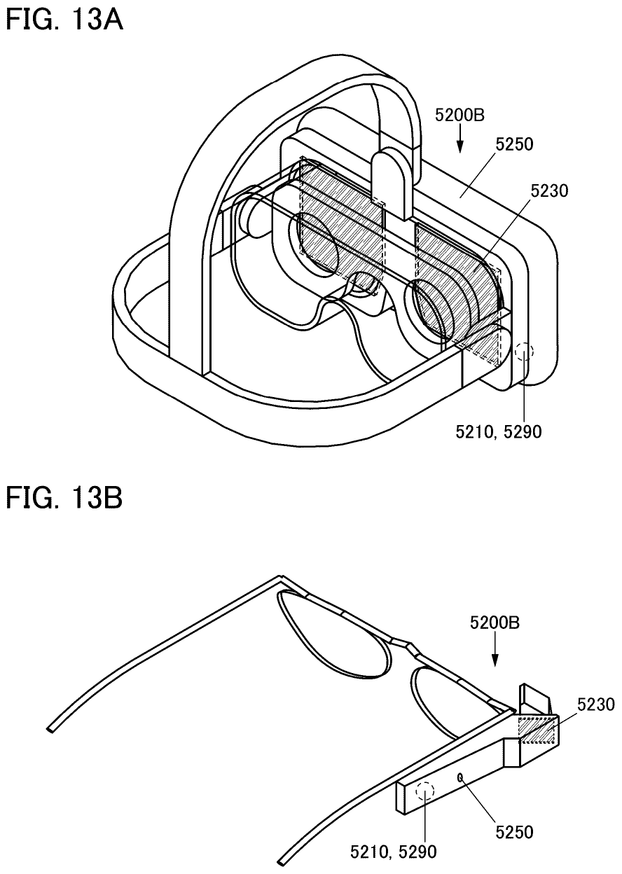

FIGS. 11A to 11E, FIGS. 12A to 12E, and FIGS. 13A and 13B each illustrate a structure of an electronic device of one embodiment of the present invention. FIG. 11A is a block diagram of an electronic device, and FIGS. 11B to 11E are perspective views illustrating structures of the electronic device. FIGS. 12A to 12E are perspective views illustrating structures of electronic devices. FIGS. 13A and 13B are perspective views illustrating structures of electronic devices.

An electronic device 5200B described in this embodiment includes an arithmetic device 5210 and an input/output device 5220 (see FIG. 11A).

The arithmetic device 5210 has a function of receiving handling data and a function of supplying image data on the basis of the handling data.

The input/output device 5220 includes a display portion 5230, an input portion 5240, a sensor portion 5250, and a communication portion 5290, and has a function of supplying handling data and a function of receiving image data. The input/output device 5220 also has a function of supplying sensing data, a function of supplying communication data, and a function of receiving communication data.

The input portion 5240 has a function of supplying handling data. For example, the input portion 5240 supplies handling data on the basis of handling by a user of the electronic device 5200B.

Specifically, a keyboard, a hardware button, a pointing device, a touch sensor, an illuminance sensor, an imaging device, an audio input device, an eye-gaze input device, an attitude sensing device, or the like can be used as the input portion 5240.

The display portion 5230 includes a display panel and has a function of displaying image data. For example, the display panel described in the above embodiment can be used for the display portion 5230.

The sensor portion 5250 has a function of supplying sensing data. For example, the sensor portion 5250 has a function of sensing a surrounding environment where the electronic device is used and supplying the sensing data.

Specifically, an illuminance sensor, an imaging device, an attitude sensing device, a pressure sensor, a human motion sensor, or the like can be used as the sensor portion 5250.

The communication portion 5290 has a function of receiving and supplying communication data. For example, the communication portion 5290 has a function of being connected to another electronic device or a communication network by wireless communication or wired communication. Specifically, the communication portion 5290 has a function of wireless local area network communication, telephone communication, near field communication, or the like.

FIG. 11B illustrates an electronic device having an outer shape along a cylindrical column or the like. An example of such an electronic device is digital signage. The display panel of one embodiment of the present invention can be used for the display portion 5230. The electronic device may have a function of changing its display method in accordance with the illuminance of a usage environment. The electronic device has a function of changing the displayed content when sensing the existence of a person. Thus, for example, the electronic device can be provided on a column of a building. The electronic device can display advertising, guidance, or the like.

FIG. 11C illustrates an electronic device having a function of generating image data on the basis of the path of a pointer used by the user. Examples of such an electronic device include an electronic blackboard, an electronic bulletin board, and digital signage. Specifically, a display panel with a diagonal size of 20 inches or longer, preferably 40 inches or longer, further preferably 55 inches or longer can be used. A plurality of display panels can be arranged and used as one display region. Alternatively, a plurality of display panels can be arranged and used as a multiscreen.

FIG. 11D illustrates an electronic device that is capable of receiving data from another device and displaying the data on the display portion 5230. An example of such an electronic device is a wearable electronic device. Specifically, the electronic device can display several options, and the user can choose some from the options and send a reply to the data transmitter. As another example, the electronic device has a function of changing its display method in accordance with the illuminance of a usage environment. Thus, for example, power consumption of the wearable electronic device can be reduced. As another example, the wearable electronic device can display an image so as to be suitably used even in an environment under strong external light, e.g., outdoors in fine weather.

FIG. 11E illustrates an electronic device including the display portion 5230 having a surface gently curved along a side surface of a housing. An example of such an electronic device is a mobile phone. The display portion 5230 includes a display panel that has a function of displaying images on the front surface, the side surfaces, the top surface, and the rear surface, for example. Thus, a mobile phone can display data not only on its front surface but also on its side surfaces, top surface, and rear surface, for example.

FIG. 12A illustrates an electronic device that is capable of receiving data via the Internet and displaying the data on the display portion 5230. An example of such an electronic device is a smartphone. For example, the user can check a created message on the display portion 5230 and send the created message to another device. As another example, the electronic device has a function of changing its display method in accordance with the illuminance of a usage environment. Thus, power consumption of the smartphone can be reduced. As another example, the smartphone can display an image so as to be suitably used even in an environment under strong external light, e.g., outdoors in fine weather.

FIG. 12B illustrates an electronic device that can use a remote controller as the input portion 5240. An example of such an electronic device is a television system. For example, data received from a broadcast station or via the Internet can be displayed on the display portion 5230. The electronic device can take an image of the user with the sensor portion 5250 and transmit the image of the user. The electronic device can acquire a viewing history of the user and provide it to a cloud service. The electronic device can acquire recommendation data from a cloud service and display the data on the display portion 5230. A program or a moving image can be displayed on the basis of the recommendation data. As another example, the electronic device has a function of changing its display method in accordance with the illuminance of a usage environment. Accordingly, the television system can display an image so as to be suitably used even under strong external light entering the room from the outside in fine weather.

FIG. 12C illustrates an electronic device that is capable of receiving an educational material via the Internet and displaying it on the display portion 5230. An example of such an electronic device is a tablet computer. The user can input an assignment with the input portion 5240 and send it via the Internet. The user can obtain a corrected assignment or the evaluation from a cloud service and have it displayed on the display portion 5230. The user can select a suitable educational material on the basis of the evaluation and have it displayed.

For example, an image signal can be received from another electronic device and displayed on the display portion 5230. When the electronic device is placed on a stand or the like, the display portion 5230 can be used as a sub-display. Thus, for example, the tablet computer can display an image so as to be suitably used even in an environment under strong external light, e.g., outdoors in fine weather.

FIG. 12D illustrates an electronic device including a plurality of the display portions 5230. An example of such an electronic device is a digital camera. For example, the display portion 5230 can display an image that the sensor portion 5250 is capturing. A captured image can be displayed on the sensor portion. A captured image can be decorated using the input portion 5240. A message can be attached to a captured image. A captured image can be transmitted via the Internet. The electronic device has a function of changing shooting conditions in accordance with the illuminance of a usage environment. Accordingly, for example, the digital camera can display a subject such that an image is suitably viewed even in an environment under strong external light, e.g., outdoors in fine weather.

FIG. 12E illustrates an electronic device in which the electronic device of this embodiment is used as a master to control another electronic device used as a slave. An example of such an electronic device is a portable personal computer. For example, part of image data can be displayed on the display portion 5230 and another part of the image data can be displayed on a display portion of another electronic device. Image signals can be supplied. Data written from an input portion of another electronic device can be obtained with the communication portion 5290. Thus, a large display region can be utilized in the case of using a portable personal computer, for example.

FIG. 13A illustrates an electronic device including the sensor portion 5250 that senses an acceleration or a direction. An example of such an electronic device is a goggles-type electronic device. The sensor portion 5250 can supply data on the position of the user or the direction in which the user faces. The electronic device can generate image data for the right eye and image data for the left eye in accordance with the position of the user or the direction in which the user faces. The display portion 5230 includes a display region for the right eye and a display region for the left eye. Thus, a virtual reality image that gives the user a sense of immersion can be displayed on the goggles-type electronic device, for example.

FIG. 13B illustrates an electronic device including an imaging device and the sensor portion 5250 that senses an acceleration or a direction. An example of such an electronic device is a glasses-type electronic device. The sensor portion 5250 can supply data on the position of the user or the direction in which the user faces. The electronic device can generate image data in accordance with the position of the user or the direction in which the user faces. Accordingly, the data can be shown together with a real-world scene, for example. Alternatively, an augmented reality image can be displayed on the glasses-type electronic device.

Note that this embodiment can be combined with any of the other embodiments in this specification as appropriate.

### Embodiment 8

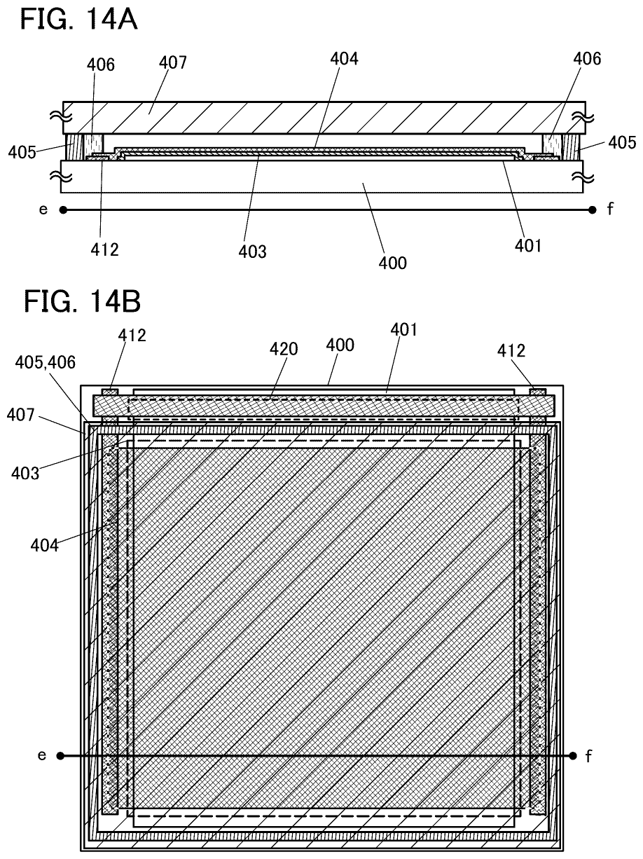

This embodiment will describe a structure in which any of the light-emitting devices described in Embodiments 2 to 4 is used as a lighting device with reference to FIGS. 14A and 14B. FIG. 14A is a cross-sectional view taken along the line e-f in a top view of the lighting device in FIG. 14B.

In the lighting device in this embodiment, a first electrode 401 is formed over a substrate 400 that is a support and has a light-transmitting property. The first electrode 401 corresponds to the first electrode 101 in Embodiment 2. When light is extracted from the first electrode 401 side, the first electrode 401 is formed using a material having a light-transmitting property.

A pad 412 for applying voltage to a second electrode 404 is provided over the substrate 400.

An EL layer 403 is formed over the first electrode 401. The structure of the EL layer 403 corresponds to the structure of the EL layer 103 in Embodiment 2. Refer to the corresponding description for these structures.

The second electrode 404 is formed to cover the EL layer 403. The second electrode 404 corresponds to the second electrode 102 in Embodiment 2. The second electrode 404 is formed using a material having high reflectance when light is extracted from the first electrode 401 side. The second electrode 404 is connected to the pad 412 so that voltage is supplied to the second electrode 404.

As described above, the lighting device described in this embodiment includes a light-emitting device including the first electrode 401, the EL layer 403, and the second electrode 404. Since the light-emitting device is any of the light-emitting devices having high emission efficiency in Embodiments 2 to 4, the lighting device in this embodiment can have low power consumption.

The substrate 400 provided with the light-emitting device having the above structure and a sealing substrate 407 are fixed and sealed with sealing materials 405 and 406, whereby the lighting device is completed. It is possible to use only either the sealing material 405 or the sealing material 406. In addition, the inner sealing material 406 (not illustrated in FIG. 14B) can be mixed with a desiccant that enables moisture to be adsorbed, leading to an improvement in reliability.

When parts of the pad 412 and the first electrode 401 are extended to the outside of the sealing materials 405 and 406, the extended parts can serve as external input terminals. An IC chip 420 mounted with a converter or the like may be provided over the external input terminals.

The structures described in this embodiment can be used in appropriate combination with any of the structures described in the other embodiments.

### Embodiment 9

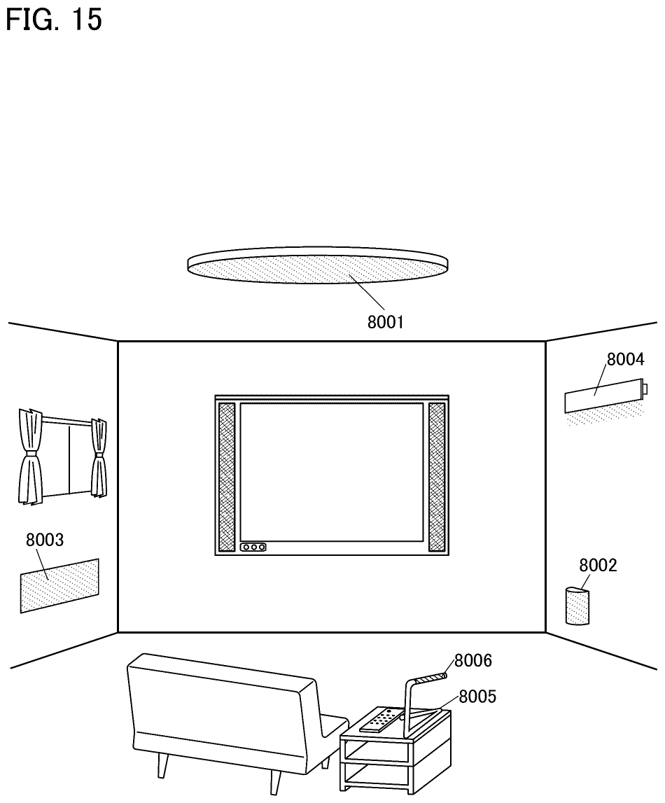

This embodiment will describe application examples of lighting devices fabricated using the light-emitting apparatus of one embodiment of the present invention or the light-emitting device, which is part of the light-emitting apparatus, with reference to FIG. 15.

A ceiling light 8001 can be used as an indoor lighting device. Examples of the ceiling light 8001 include a direct-mount light and an embedded light. Such lighting devices are fabricated using the light-emitting apparatus in combination with a housing and a cover. Application to a cord pendant light (light that is suspended from a ceiling by a cord) is also possible.

Afoot light 8002 lights a floor so that safety on the floor can be improved. For example, it can be effectively used in a bedroom, on a staircase, and on a passage. In such cases, the size and shape of the foot light can be changed in accordance with the dimensions and structure of a room. The foot light can be a stationary lighting device using the light-emitting apparatus and a support in combination.

A sheet-like lighting 8003 is a thin sheet-like lighting device. The sheet-like lighting, which is attached to a wall when used, is space-saving and thus can be used for a wide variety of uses. Furthermore, the area of the sheet-like lighting can be easily increased. The sheet-like lighting can also be used on a wall, a housing, or the like that has a curved surface.

A lighting device 8004 in which the direction of light from a light source is controlled to be only a desired direction can be used.

A desk lamp 8005 includes a light source 8006. As the light source 8006, the light-emitting apparatus of one embodiment of the present invention or the light-emitting device, which is part of the light-emitting apparatus, can be used.

Besides the above examples, when the light-emitting apparatus of one embodiment of the present invention or the light-emitting device, which is part of the light-emitting apparatus, is used as part of furniture in a room, a lighting device that functions as the furniture can be obtained.

As described above, a variety of lighting devices that include the light-emitting apparatus can be obtained. Note that these lighting devices are also embodiments of the present invention.

The structures described in this embodiment can be used in appropriate combination with any of the structures described in the other embodiments.

### Example

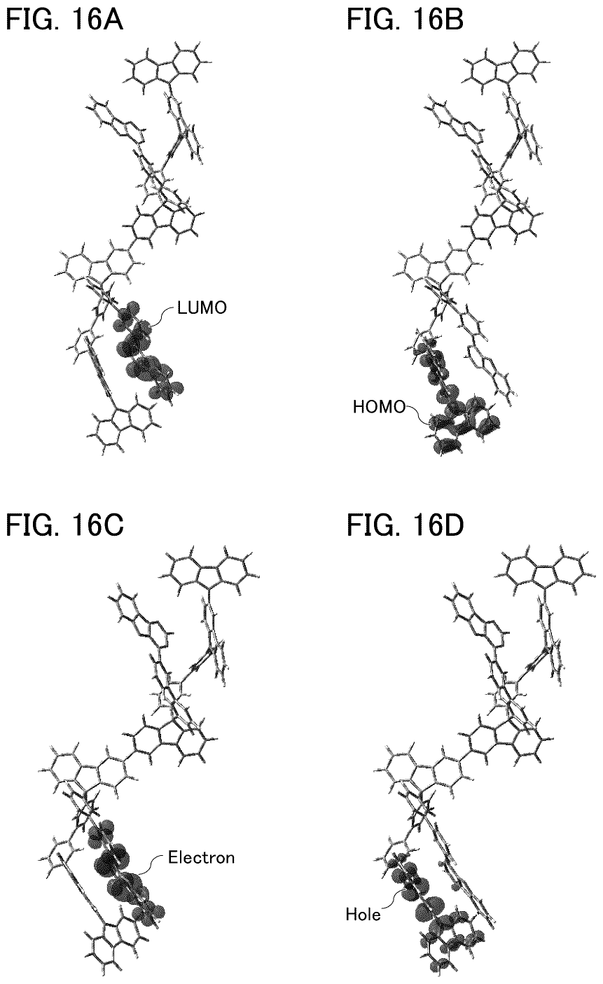

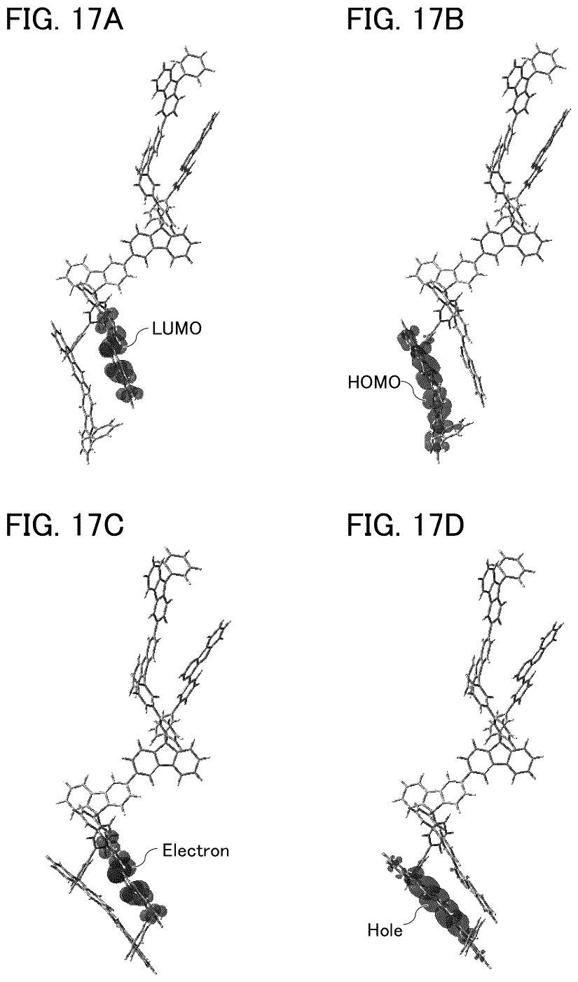

In this example, the high molecular compound of one embodiment of the present invention was analyzed by calculation, and the results are described with reference to FIGS. 16A to 16D and FIGS. 17A to 17D. In this example, dimers of the repeating unit included in the high molecular compound of one embodiment of the present invention were analyzed.

Chemical formulae of Dimer A and Dimer B, which were subjected to analysis, are shown below.

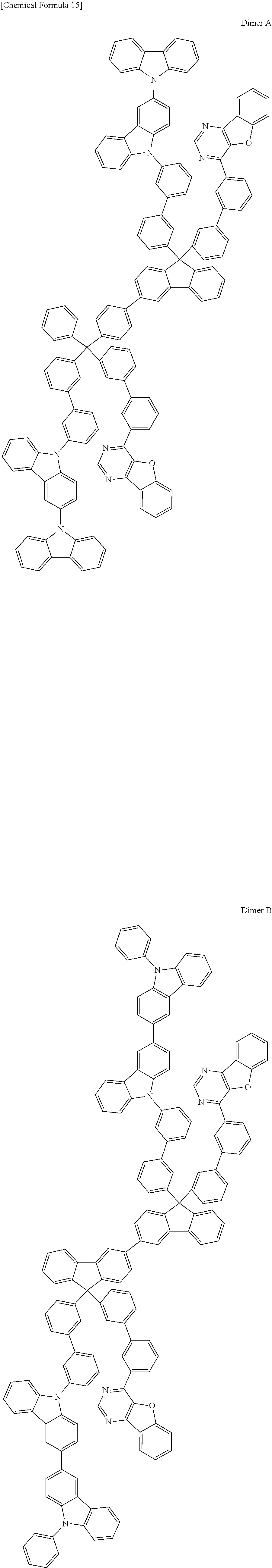

### <Structural Analysis by Calculation>

The HOMO and LUMO distributions in the most stable structure where the singlet ground state (S₀) level of a compound is the lowest were analyzed. In addition, the hole and electron distributions in the most stable structure where the triplet excited state (T₁) level of the compound is the lowest were analyzed. A density functional theory (DFT) method was used as the calculation method. The total energy calculated by the DFT is represented as the sum of potential energy, electrostatic energy between electrons, electronic kinetic energy, and exchange-correlation energy including all the complicated interactions between electrons. In the DFT, an exchange-correlation interaction is approximated by a functional (a function of another function) of one electron potential represented in terms of electron density to enable high-speed calculations. Here, CAM-B3LYP which is a hybrid functional was used to specify the weight of each parameter related to exchange-correlation energy. As a basis function, 6-311G (d,p) was used. Gaussian 16 was used as a computational program.

In this specification and the like, a partial structure on which the HOMO and LUMO distributions are located refers to a partial structure on which molecular orbital distribution is located when the absolute value of the threshold for displaying the molecular orbital is 0.03 (e-bohr⁻³)^{1/2} in the most stable structure where the S₀ level is the lowest. In addition, a structure on which the hole and electron distributions are located refers to a partial structure on which molecular orbital distribution is located when the absolute value of the threshold for displaying the LUMO of p electrons and the HOMO of a electrons is 0.03 (e-bohr⁻³)^{1/2} in the most stable structure where the T₁ level is the lowest.

FIGS. 16A to 16D and FIGS. 17A to 17D show the analysis results of Dimer A and Dimer B. Shades around molecules in FIGS. 16A to 16D and FIGS. 17A to 17D indicate the HOMO, LUMO, hole, or electron distribution in the molecules.

FIG. 16A shows the LUMO distribution in the S₀ state of Dimer A, FIG. 16B shows the HOMO distribution in the S₀ state of Dimer A, FIG. 16C shows the electron distribution in the T₁ state of Dimer A, and FIG. 16D shows the hole distribution in the T₁ state of Dimer A. FIG. 17A shows the LUMO distribution in the S₀ state of Dimer B, FIG. 17B shows the HOMO distribution in the S₀ state of Dimer B, FIG. 17C shows the electron distribution in the T₁ state of Dimer B, and FIG. 17D shows the hole distribution in the T₁ state of Dimer B.

FIGS. 16A and 16B reveal that, in the S₀ state of Dimer A, the LUMO distribution is located on a benzofuropyrimidine skeleton that is an electron-transport skeleton, and the HOMO distribution is located on a carbazole skeleton that is a hole-transport skeleton. It is thus found that the HOMO distribution and the LUMO distribution are separated from each other in the S₀ state of Dimer A. FIGS. 16C and 16D reveal that, in the T₁ state of Dimer A, the electron distribution is located on the benzofuropyrimidine skeleton that is the electron-transport skeleton, and the hole distribution is located on the carbazole skeleton that is the hole-transport skeleton. It is thus found that intramolecular charge transfer occurs between the hole-transport skeleton and the electron-transport skeleton in the T₁ state of Dimer A.

FIGS. 17A and 17B reveal that, in the S₀ state of Dimer B, the LUMO distribution is located on a benzofuropyrimidine skeleton that is an electron-transport skeleton, and the HOMO distribution is located on a carbazole skeleton that is a hole-transport skeleton. It is thus found that the HOMO distribution and the LUMO distribution are separated from each other in the S₀ state of Dimer B. FIGS. 17C and 17D reveal that, in the T₁ state of Dimer B, the electron distribution is located on the benzofuropyrimidine skeleton that is the electron-transport skeleton, and the hole distribution is located on the carbazole skeleton that is the hole-transport skeleton. It is thus found that intramolecular charge transfer occurs between the hole-transport skeleton and the electron-transport skeleton in the T₁ state of Dimer B.

The above results demonstrate that the intramolecular charge transfer occurs between the hole-transport skeleton and the electron-transport skeleton in the excited state in each of the dimers of the repeating unit included in the high molecular compound of one embodiment of the present invention. Thus, intramolecular charge transfer probably occurs between the hole-transport skeleton and the electron-transport skeleton in the excited state also in the high molecular compound of one embodiment of the present invention. Accordingly, it is found that with the use of the high molecular compound of one embodiment of the present invention, a highly efficient light-emitting device can be provided.

This application is based on Japanese Patent Application Serial No. 2022-filed with Japan Patent Office on Sep. 22, 2022, the entire contents of which are hereby incorporated by reference.

What is claimed is:

1. A high molecular compound comprising:
a repeating unit,
wherein the repeating unit comprises a fluorenediyl group, a hole-transport skeleton, and an electron-transport skeleton,
wherein the hole-transport skeleton is bonded to the fluorenediyl group through a substituted or unsubstituted first arylene group,
wherein the electron-transport skeleton is bonded to the fluorenediyl group through a substituted or unsubstituted second arylene group, and
wherein intramolecular charge transfer occurs between the hole-transport skeleton and the electron-transport skeleton in an excited state.

2. The high molecular compound according to claim 1,
wherein the first arylene group and the second arylene group are bonded to carbon at a 9-position of the fluorenediyl group.

3. The high molecular compound according to claim 1,
wherein at least one of the first arylene group and the second arylene group is either a substituted or unsubstituted m-phenylene group or a group in which a plurality of substituted or unsubstituted m-phenylene groups are bonded to one another.

4. A high molecular compound comprising a repeating unit represented by Formula (G1): 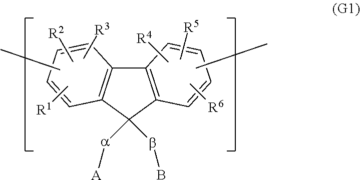
wherein α and β each independently represent a substituted or unsubstituted arylene group having 6 to 24 carbon atoms,
wherein A represents a hole-transport skeleton,
wherein B represents an electron-transport skeleton, and
wherein R¹ to R⁶ each independently represent any one of hydrogen, a straight-chain alkyl group having 1 to 10 carbon atoms, a branched alkyl group having 3 to 10 carbon atoms, an alkoxy group having 1 to 10 carbon atoms, a cycloalkyl group having 3 to 12 carbon atoms, a cycloalkyl group having a bridged structure and having 4 to 10 carbon atoms, and a trialkylsilyl group having 3 to 12 carbon atoms.

5. The high molecular compound according to claim 4,
wherein α and β each independently represent either a substituted or unsubstituted m-phenylene group or a group in which a plurality of substituted or unsubstituted m-phenylene groups are bonded to one another.

6. The high molecular compound according to claim 1,
wherein the hole-transport skeleton comprises at least one of a π-electron rich heteroaromatic skeleton and an aromatic amine skeleton, and
wherein the electron-transport skeleton comprises a π-electron deficient heteroaromatic skeleton.

7. A light-emitting device comprising:
the high molecular compound according to claim 1; and
a light-emitting substance.

8. A light-emitting apparatus comprising:
the light-emitting device according to claim 7; and
at least one of a transistor and a substrate.

9. An electronic device comprising:
the light-emitting apparatus according to claim 8; and
at least one of a sensing portion, an input portion, and a communication portion.

10. A lighting device comprising:
the light-emitting apparatus according to claim 8; and
a housing.

11. The high molecular compound according to claim 4,
wherein the hole-transport skeleton comprises at least one of a π-electron rich heteroaromatic skeleton and an aromatic amine skeleton, and
wherein the electron-transport skeleton comprises a π-electron deficient heteroaromatic skeleton.

12. A light-emitting device comprising:
the high molecular compound according to claim 4; and
a light-emitting substance.

13. A light-emitting apparatus comprising:
the light-emitting device according to claim 12; and
at least one of a transistor and a substrate.

14. An electronic device comprising:
the light-emitting apparatus according to claim 13; and
at least one of a sensing portion, an input portion, and a communication portion.

15. A lighting device comprising:
the light-emitting apparatus according to claim 13; and
a housing.
